(19) United States
(12) Patent Application Publication
(10) Pub. No.: US 2025/0111935 A1
(43) Pub. Date: Apr. 3, 2025

(54) CONFIGURABLE SYSTEM FOR MEDICAL PROCESSING OPERATIONS

(71) Applicant: Translational Imaging Innovations, Inc., Hickory, NC (US)

(72) Inventors: Eric L. Buckland, Hickory, NC (US); Alexander E. Salmon, Garner, NC (US)

(21) Appl. No.: 18/902,057

(22) Filed: Sep. 30, 2024

Related U.S. Application Data
(60) Provisional application No. 63/586,782, filed on Sep. 29, 2023.
(60) Provisional application No. 63/587,497, filed on Oct. 3, 2023.

(51) Int. Cl. G16H 30/40; G16H 10/20; G16H 10/60; G16H 20/00; G16H 40/20

(52) U.S. Cl. CPC G16H 30/40; G16H 10/20; G16H 10/60; G16H 20/00; G16H 40/20

(57) ABSTRACT

Disclosed herein is configurable medical data processing workflow, including a set of configurable processing operations that can be applied to medical imaging data. The operations can be configured based on user-defined criteria, such as selecting predefined operations, adjusting parameters, or defining custom tasks. The medical data processing workflow can facilitate the execution of automated and/or operator-assisted tasks, each resulting in a transformation or annotation of the medical data. Records can be generated for each task, including unique identifiers and operation details. A traceability report can be generated, documenting all operations performed, enabling verification and traceability of the data processing path, supporting data integrity and compliance.

### RELATED APPLICATIONS

Any and all applications for which a foreign or domestic priority claim is identified in the Application Data Sheet as filed with the present application are incorporated by reference under 37 CFR 1.57 and made a part of this specification. This application claims priority to U.S. Provisional Patent App. No. 63/586,782, filed Sep. 29, 2023, entitled “Process Automation For Hybrid Robotic Image Analysis Workflows” and U.S. Provisional Patent App. No. 63/587,497, filed Oct. 3, 2023, entitled “Cone Metrics as Biomarker for Patient Selection in Clinical Trials,” each of which is hereby incorporated by reference in its entirety.

### FIELD

The present disclosure generally relates to medical data processing systems and, more specifically, to configurable medical processing operations, with traceability of data transformations, annotations, computations, and interpretations.

### BACKGROUND

In medical processes such as clinical research, diagnostics, and treatment planning, various types of medical information—such as imaging data and test results—are processed through multiple stages, including acquisition, transformation, annotation, or analysis, with the aim of extracting meaningful insights. These insights can be used to assess patient conditions, track disease progression, identify biomarkers, or evaluate the effectiveness of treatments. These processes often include a combination of automated systems, expert input, and supporting tools such as data integration platforms, statistical analysis tools, and image segmentation software, all while complying with regulatory standards for managing sensitive information.

Traditionally, many of these processes are fragmented, with certain tasks being handled in isolation. For instance, different systems might be used for acquiring data, annotating images, analyzing results, or managing compliance, and expert annotations can be handled separately. This separation can create challenges with traceability, where it may be unclear exactly what modifications or transformations have been applied to the data throughout the process. This lack of traceability can present issues in clinical trials and other regulated environments, where having thorough documentation and verification of each step in the data handling process can be important for supporting compliance and reproducibility.

Manual input, particularly expert-driven annotation and review, can play a role in interpreting complex images or validating computational outputs. However, integrating these human-driven tasks with fully automated, machine-driven tasks is often lacking, leading to inefficiencies in data traceability and integrity. This also hinders the system's ability to balance human expertise with automated efficiencies. While automated systems can improve efficiency by managing repetitive tasks, they are often not fully integrated with manual processes or other tools such as machine learning algorithms, collaborative platforms, or data visualization systems. This lack of integration can make it difficult to maintain a clear, traceable record of how and when changes occur, which can be useful for ensuring data integrity and regulatory compliance. Furthermore, as artificial intelligence and machine learning become more prevalent in medical processes, this fragmentation can further complicate efforts to streamline operations and enhance the effectiveness of clinical research, diagnostics, and treatment planning.

### SUMMARY

Disclosed herein is configurable medical data processing workflow, including a set of configurable processing operations that can be applied to medical imaging data. The operations can be configured based on user-defined criteria, such as selecting predefined operations, adjusting parameters, or defining custom tasks. The medical data processing workflow can facilitate the execution of automated and/or operator-assisted tasks, each resulting in a transformation or annotation of the medical data. Records can be generated for each task, including unique identifiers and operation details. A traceability report can be generated, documenting all operations performed, enabling verification and traceability of the data processing path, supporting data integrity and compliance.

Certain illustrative examples are described in the following numbered clauses:

Clause 1. A method for managing a configurable medical data processing workflow, the method comprising:
providing a medical data processing workflow comprising a set of configurable processing operations, wherein the medical data processing workflow is applied to process medical imaging data;
configuring one or more processing operations of the set of configurable processing operations based on user-defined criteria, wherein the user-defined criteria comprises at least one of:
   a selection of at least one processing operation from a plurality of predefined processing operations,
   an indication of an adjustment of one or more parameters associated with the selected processing operation, or
   an indication of a user-defined processing operation;
facilitating execution of the set of configurable processing operations on the medical imaging data to generate an output, wherein the execution includes performing a plurality of tasks, the plurality of tasks comprising at least one computer-automated task and at least one operator-assisted task, wherein each task of the plurality of tasks results in at least one of a transformation or an annotation of at least a portion of the medical imaging data;
generating a record for each task of the plurality of tasks, wherein each record includes a unique identifier corresponding to the task and details of a respective transformation or annotation applied to the medical imaging data; and
generating a traceability report based on the plurality of records, wherein the traceability report provides a complete record of all processing operations performed on the medical imaging data, including a sequence of operations, unique identifiers for each operation, details of any transformation or annotation applied to the medical imaging data, and identification of the operator responsible for each task, such that the report enables the tracing of each modification or annotation back to its corresponding step in the medical data processing workflow, ensuring that the entire data processing path is documented and verifiable.

Clause 2. The method of clause 1, wherein the set of configurable processing operations and user-defined criteria form an ontology, the ontology comprising a structured framework that defines relationships between data entities.

Clause 3. The method of any of the previous clauses, wherein the configuring of the one or more processing operations comprises user-configuration through modular low-code or no-code interaction, the modular low-code or no-code interaction including graphical user interface elements associated with modules of an ontology, enabling selection and/or adjustment of workflow operations without requiring detailed coding.

Clause 4. The method of any of the previous clauses, wherein each configurable processing operation is assigned a universally unique identifier (UUID) and is stored as a reusable configuration, such that the processing operation can be reapplied in subsequent workflows.

Clause 5. The method of any of the previous clauses, wherein a complete set of user-configurations that form the medical data processing workflow is assigned a universally unique identifier (UUID), enabling a complete version of the medical data processing workflow to be saved and reused in future instances of medical data processing.

Clause 6. The method of any of the previous clauses, further comprising causing a display to present graphical user interface elements, each graphical user interface element corresponding to a particular configurable processing operation from the set of configurable processing operations, wherein the graphical user interface elements enable selection, adjustment of parameters, or definition of a user-defined processing operation for inclusion in the medical data processing workflow.

Clause 7. The method of any of the previous clauses, further comprising facilitating human interaction with the medical data processing workflow through graphical user interface elements, wherein the human interaction includes at least one of reviewing, annotating, or adjusting the configurable processing operations based on clinical or operational criteria, and wherein the human interaction is recorded as part of the traceability report.

Clause 8. The method of any of the previous clauses, wherein the traceability report further includes audit logs of user interactions, wherein each interaction is logged with a unique identifier and user credentials for full accountability.

Clause 9. The method of any of the previous clauses, further comprising assigning a universally unique identifier (UUID) to the medical imaging data at an initial stage of the workflow, wherein at each subsequent step of the configurable processing workflow, as the data is transformed, annotated, or divided into sub-portions, each resulting portion or subset of the data is assigned an additional UUID, such that the data forms a branching sequence with a unique identifier at each branch, providing traceability for every division and modification of the data throughout the workflow.

Clause 10. The method of any of the previous clauses, wherein the medical data processing workflow is applied to evaluate a set of prospective biomarkers, wherein the medical data processing workflow comprises computing a set of metrics associated with a set of locations within an image or a test result, associating the metrics with at least one record of patient metadata, and developing a classification schema for sorting patients by categories of the metadata value using a combination of one or more metrics associated with one or more locations in the test region of the patient.

Clause 11. The method of Clause 10, wherein the medical data processing workflow comprises computing a set metrics, reducing the set of metrics to a predefined set of one or more biomarkers, applying a candidate patient data set to the biomarker workflow, and classifying the candidate patient eligibility for participation in a clinic trial or eligibility to receive a clinical treatment.

Clause 12. The method of any of the previous clauses, wherein the medical data processing workflow is configured for reading medical data in a clinical research study or a clinical trial.

Clause 13. The method of any of the previous clauses, wherein the medical data processing workflow comprises automatedly creating masked and randomized batches of images, annotating the masked and randomized batches of images, automatedly computing a set of metrics from the images in the masked and randomized batches, and automatedly creating a report on computed set of metrics.

Clause 14. The method of any of the previous clauses, wherein the at least one operator-assisted task comprises at least one of selecting, adjusting, or confirming processing operations.

Clause 15. The method of any of the previous clauses, wherein the traceability report further comprises a hierarchical structure that organizes each task of the plurality of tasks and any derived sub-tasks based on a parent-child relationship, wherein each task and sub-task is assigned a universally unique identifier, such that the hierarchical structure enables tracking and tracing of the processing operations across multiple branches within the medical data processing workflow.

Clause 16. The method of any of the previous clauses, wherein the universally unique identifier assigned to each task of the plurality of tasks within the medical data processing workflow is linked to subsequent sub-tasks generated from the transformation or annotation of the medical imaging data, such that a hierarchical structure of the traceability report provides a detailed, reproducible path of all tasks and sub-tasks performed within the workflow.

Clause 17. The method of any of the previous clauses, wherein the hierarchical structure of the traceability report further enables recreation of a complete medical data processing workflow, such that by following the universally unique identifiers and recorded sequence of tasks, the medical data processing operations can be reproduced to generate an output identical to the original workflow execution.

Clause 18. A computer-readable medium storing instructions that, when executed by one or more processors, cause the one or more processors to:
provide a medical data processing workflow comprising a set of configurable processing operations, wherein the medical data processing workflow is applied to process medical imaging data;
configure one or more processing operations of the set of configurable processing operations based on user-defined criteria, wherein the user-defined criteria comprises at least one of:
   a selection of at least one processing operation from a plurality of predefined processing operations,
   an indication of an adjustment of one or more parameters associated with the selected processing operation, or
   an indication of a user-defined processing operation;
facilitate execution of the set of configurable processing operations on the medical imaging data to generate an output, wherein the execution includes performing a plurality of tasks, the plurality of tasks comprising at least one computer-automated task and at least one operator-assisted task, wherein each task of the plurality of tasks results in at least one of a transformation or an annotation of at least a portion of the medical imaging data;
generate a record for each task of the plurality of tasks, wherein each record includes a unique identifier corresponding to the task and details of a respective transformation or annotation applied to the medical imaging data; and
generate a traceability report based on the plurality of records, wherein the traceability report provides a complete record of all processing operations performed on the medical imaging data, including a sequence of operations, unique identifiers for each operation, details of any transformation or annotation applied to the medical imaging data, and identification of the operator responsible for each task, such that the report enables the tracing of each modification or annotation back to its corresponding step in the medical data processing workflow, ensuring that the entire data processing path is documented and verifiable.

Clause 19. The computer-readable medium of clause 18, wherein the configuring of the one or more processing operations comprises user-configuration through modular low-code or no-code interaction, the modular low-code or no-code interaction including graphical user interface elements associated with modules of an ontology, enabling selection and/or adjustment of workflow operations without requiring detailed coding.

Clause 20. A system comprising one or more processors configured to:
provide a medical data processing workflow comprising a set of configurable processing operations, wherein the medical data processing workflow is applied to process medical imaging data;
configure one or more processing operations of the set of configurable processing operations based on user-defined criteria, wherein the user-defined criteria comprises at least one of:
   a selection of at least one processing operation from a plurality of predefined processing operations,
   an indication of an adjustment of one or more parameters associated with the selected processing operation, or
   an indication of a user-defined processing operation;
facilitate execution of the set of configurable processing operations on the medical imaging data to generate an output, wherein the execution includes performing a plurality of tasks, the plurality of tasks comprising at least one computer-automated task and at least one operator-assisted task, wherein each task of the plurality of tasks results in at least one of a transformation or an annotation of at least a portion of the medical imaging data;
generate a record for each task of the plurality of tasks, wherein each record includes a unique identifier corresponding to the task and details of a respective transformation or annotation applied to the medical imaging data; and
generate a traceability report based on the plurality of records, wherein the traceability report provides a complete record of all processing operations performed on the medical imaging data, including a sequence of operations, unique identifiers for each operation, details of any transformation or annotation applied to the medical imaging data, and identification of the operator responsible for each task, such that the report enables the tracing of each modification or annotation back to its corresponding step in the medical data processing workflow, ensuring that the entire data processing path is documented and verifiable.

Clause 21. A method for determining an eligibility status of an individual for participation in a clinical trial for treating degenerative retinal diseases, the method comprising:
obtaining retinal image data of an eye of an individual, wherein the retinal image data reflects a topographic structure of cone photoreceptors in the eye;
analyzing the retinal image data to compute at least one quantitative metric related to cone photoreceptor distribution, the at least one quantitative metric comprising a cone density metric, a cone spacing metric, or a regularity metric of cone packing;
comparing the at least one quantitative metric with a predefined threshold indicative of retinal degeneration progression;
stratifying the individual into an inclusion or exclusion category for a clinical trial based on the comparing; and
determining an eligibility status for the individual based on the stratifying, wherein the individual is determined to be eligible for inclusion in the clinical trial if the at least one quantitative metric satisfies the predefined threshold, indicating therapeutic potential.

Clause 22. The method of clause 21, wherein the analyzing comprises computing the quantitative metric as a function of distance from a fovea of the eye.

Clause 23. The method of clause 21, wherein the analyzing comprises calculating the quantitative metric by determining distances between adjacent cone photoreceptors within a defined region of interest in a retina of the eye.

Clause 24. The method of clause 23, wherein the quantitative metric is computed by detecting cone photoreceptor locations using a convolutional neural network trained on retinal image datasets.

Clause 25. The method of clause 21, wherein the analyzing comprises determining the regularity metric of cone packing by evaluating a geometric arrangement of the cone photoreceptors in the eye, based on variations in the cone packing metric.

Clause 26. The method of clause 21, wherein the analyzing further comprises identifying regions of abnormal cone photoreceptor distribution within a retina of the eye, based on a deviation of the at least one quantitative metric from a normative dataset of healthy individuals.

Clause 27. The method of clause 21, wherein the at least one quantitative metric comprises the cone density metric, the cone spacing metric, and the regularity metric of cone packing.

Clause 28. The method of clause 21, further comprising determining a severity of retinal degeneration progression by comparing the at least one quantitative metric with multiple predefined thresholds indicative of different stages of the disease, wherein the stratifying is based on the determined severity.

Clause 29. The method of clause 21, wherein the analyzing comprises employing machine learning algorithms to classify retinal images and predict retinal degeneration progression based on patterns in the cone photoreceptor distribution.

Clause 30. The method of clause 21, wherein the analyzing further comprises:
generating a spatial heat map representing density and spacing of cone photoreceptors across different regions of a retina of the eye; and
outputting an indication of the spatial heat map, the spatial heat map being usable to identify localized areas of degeneration.

Clause 31. The method of clause 21, further comprising:
implementing a configurable workflow for analyzing the retinal image data, wherein the workflow includes a set of user-configurable steps for computing the at least one quantitative metric related to cone photoreceptor distribution;
configuring the workflow based on user-defined criteria, wherein the user-defined criteria include selecting specific analysis methods for computing cone density, cone spacing, or regularity of cone packing, and adjusting parameters associated with the selected analysis methods;
executing the configured workflow to analyze the retinal image data and compute the at least one quantitative metric; and
generating a traceability report documenting the configured steps, including any analysis methods and parameters used, for verifying the determination of the eligibility status.

Clause 32. A computer-readable medium storing instructions that, when executed by one or more processors, cause the one or more processors to:
obtain retinal image data of an eye of an individual, wherein the retinal image data reflects a topographic structure of cone photoreceptors in the eye;
analyze the retinal image data to compute at least one quantitative metric related to cone photoreceptor distribution, the at least one quantitative metric comprising a cone density metric, a cone spacing metric, or a regularity metric of cone packing;
compare the at least one quantitative metric with a predefined threshold indicative of retinal degeneration progression;
stratify the individual into an inclusion or exclusion category for a clinical trial based on the comparison; and
determine an eligibility status for the individual based on the stratifying, wherein the individual is determined to be eligible for inclusion in the clinical trial if the at least one quantitative metric satisfies the predefined threshold, indicating therapeutic potential.

Clause 33. The computer-readable medium of clause 32, wherein analyzing comprises computing the quantitative metric as a function of distance from a fovea of the eye.

Clause 34. The computer-readable medium of clause 32, wherein analyzing calculating the quantitative metric by determining distances between adjacent cone photoreceptors within a defined region of interest in a retina of the eye.

Clause 35. The computer-readable medium of clause 32, wherein analyzing determining the regularity metric of cone packing by evaluating a geometric arrangement of the cone photoreceptors in the eye, based on variations in the cone packing metric.

Clause 36. The computer-readable medium of clause 32, wherein the at least one quantitative metric comprises the cone density metric, the cone spacing metric, and the regularity metric of cone packing.

Clause 37. The computer-readable medium of clause 32, wherein the instructions, when executed by the one or more processors, further cause the one or more processors to:
implement a configurable workflow for analyzing the retinal image data, wherein the workflow includes a set of user-configurable steps for computing the at least one quantitative metric related to cone photoreceptor distribution;
configure the workflow based on user-defined criteria, wherein the user-defined criteria include selecting specific analysis methods for computing cone density, cone spacing, or regularity of cone packing, and adjusting parameters associated with the selected analysis methods;
execute the configured workflow to analyze the retinal image data and compute the at least one quantitative metric; and
generate a traceability report documenting the configured steps, including any analysis methods and parameters used, for verifying the determination of the eligibility status.

Clause 38. A system for determining an eligibility status of an individual for participation in a clinical trial for treating degenerative retinal diseases, the system comprising:
one or more processors configured to:
obtain retinal image data of an eye of an individual, wherein the retinal image data reflects a topographic structure of cone photoreceptors in the eye;
analyze the retinal image data to compute at least one quantitative metric related to cone photoreceptor distribution, the at least one quantitative metric comprising a cone density metric, a cone spacing metric, or a regularity metric of cone packing;
compare the at least one quantitative metric with a predefined threshold indicative of retinal degeneration progression;
stratify the individual into an inclusion or exclusion category for a clinical trial based on the comparison; and
determine an eligibility status for the individual based on the stratifying, wherein the individual is determined to be eligible for inclusion in the clinical trial if the at least one quantitative metric satisfies the predefined threshold, indicating therapeutic potential.

Clause 39. The system of Clause 38, wherein the at least one quantitative metric comprises the cone density metric, the cone spacing metric, and the regularity metric of cone packing.

Clause 40. The system of Clause 38, wherein the one or more processors are further configured to:
implement a configurable workflow for analyzing the retinal image data, wherein the workflow includes a set of user-configurable steps for computing the at least one quantitative metric related to cone photoreceptor distribution;
configure the workflow based on user-defined criteria, wherein the user-defined criteria include selecting specific analysis methods for computing cone density, cone spacing, or regularity of cone packing, and adjusting parameters associated with the selected analysis methods;
execute the configured workflow to analyze the retinal image data and compute the at least one quantitative metric; and
generate a traceability report documenting the configured steps, including any analysis methods and parameters used, for verifying the determination of the eligibility status.

### BRIEF DESCRIPTION OF THE DRAWINGS

Throughout the drawings, reference numbers can be re-used to indicate correspondence between referenced elements. The drawings are provided to illustrate embodiments of the present disclosure and do not limit the scope thereof.

FIG. 1 illustrates an example workflow in accordance with some embodiments of the inventive concepts, showing stages for ingesting images and data, curating and annotating images and data, and performing computations.

FIG. 2 illustrates an example workflow environment configured to manage and process medical imaging data, in accordance with some embodiments of the inventive concepts.

FIG. 3 illustrates an example cloud-based architecture for data and workflow management in accordance with some embodiments of the inventive concepts.

FIG. 4 illustrates a hierarchical organization of entities for managing data related to medical research, clinical trials, or clinical evaluations.

FIG. 5 illustrates an example workflow for the analysis of retinal images in a clinical research or trial environment.

FIG. 6 illustrates an example dashboard interface for tracking site information, personnel certifications, and stages of image analysis.

FIG. 7 illustrates an example user interface for selecting a Package type and submitting it with predefined rules and content requirements.

FIG. 8 illustrates an example real-world image processing workflow according to some embodiments of the inventive concepts.

FIG. 9 illustrates an example image processing workflow involving the selection and extraction of regions of interest (ROIs) from image montages.

FIG. 10 illustrates the beginning of a robotic batch process automation (RBPA) after the extraction of ROIs from image montages.

FIG. 11 illustrates a flow chart of an example batch allocation algorithm with user-settable parameters in accordance with some embodiments of the inventive concepts.

FIG. 12 illustrates a concept of collections and projects extended to support functionality within a workflow in accordance with some embodiments of the inventive concepts.

FIG. 13 illustrates a linearized process flow reflective of an example workflow embodiment in accordance with some embodiments of the inventive concepts.

FIG. 14 illustrates an example display of the main user interface screen for a workflow management system.

FIGS. 15A-15C illustrate example administrative pages for adding new users, including role assignment and site access configuration.

FIGS. 16A and 16B illustrate screenshots of participating sites and an example interface for submitting site information, respectively.

FIG. 17 illustrates an example user interface screenshot for submitting an imaging visit Package in the workflow system.

FIG. 18 illustrates an example step in the retina imaging process, involving the extraction and submission of ROIs from a visit montage, in accordance with some embodiments of the inventive concepts.

FIG. 19 illustrates an example user interface screenshot for uploading a Package of batched ROIs in accordance with some embodiments of the inventive concepts.

FIG. 20 illustrates an example screenshot for uploading a Package of metrics derived from the grading of ROIs.

FIG. 21 illustrates example transaction histories available through a sortable view in an example workflow management system, in accordance with some embodiments of the inventive concepts.

FIG. 22 illustrates an example dashboard with a series of Kanban cards representing different stages of an image processing workflow, in accordance with some embodiments of the inventive concepts.

FIG. 23 illustrates an organizational user interface for constructing custom workflow elements in a system, in accordance with some embodiments of the inventive concepts.

FIG. 24 illustrates example Package Upload requirements stack and includes components for setting File Requirements, Metadata Requirements, Authorizations, and Reminders.

FIG. 25 illustrates example Package Download requirements, including password protections, filters, and authorizations.

FIG. 26 illustrates example Package Review requirements, including flag for requiring a review, setting default reviewers and number of approvals required, setting authorizations, and scheduling reminders.

FIGS. 27 and 28 shows example Package Appending and Replacing requirements, respectively, include flags for allowing content appending/replacing, and setting authorizations.

FIG. 29 shows example Workflow Advancing requirements including specifying the default assignees, number of assignees, authorization, and scheduling reminders.

FIG. 30 shows an example workflow ordering model, in accordance with some embodiments of the inventive concepts.

FIGS. 31 and 32 illustrate example UI design elements including setting validation rules for single-step and multi-step workflows, respectively.

FIG. 33 illustrates example UI element for establishing new Teams.

FIG. 34 illustrates an example UI element for filling in missing metadata at a submittal step.

FIG. 35 is a flow diagram illustrative of an embodiment of a routine for managing a configurable medical data processing workflow.

FIGS. 36A-36C depict retinal images representing cone photoreceptor topography at varying levels of structural integrity, which can be utilized to compute quantitative metrics related to cone photoreceptor distribution.

FIG. 37A depicts a cross-sectional optical coherence tomography (OCT) image of the retina, highlighting the structural layers of the retina in a subject with Blue Cone Monochromacy (BCM).

FIG. 37B shows a high-resolution retinal image captured using adaptive optics (AO) imaging.

FIGS. 38A and 38B depict high-resolution adaptive optics (AO) retinal images of two patients with achromatopsia, a condition characterized by the absence of cone function.

FIGS. 39A-39K illustrate different stages of the biomarker development process for analyzing cone photoreceptor topography, progressing from imaging through computation and classification to final analysis.

FIG. 40 is an illustration of various retinal domains, with the large circular area outlining the macula.

FIG. 41 illustrates a grid in an embodiment of the present invention that is used to localize retinal sectors of interest for analysis of the retinal metrics.

FIG. 42 is a table illustrative of five retinal domains: the umbo, foveola, fovea, parafovea, and perifovea, as a function of eccentricity radius.

FIGS. 43A-47B illustrate key results from our retrospective study on markers of various cone mediated diseases, with examples of photoreceptor metrics and their spatial distribution as functions of both foveal eccentricity and meridian sector, for healthy controls and individuals diagnosed with retinal diseases.

FIG. 48 is a flow diagram illustrative of an embodiment of a routine for determining the eligibility of an individual for a clinical trial.

FIG. 49 is a flow diagram illustrative of an embodiment of a routine for determining the eligibility of an individual for a clinical trial or treatment.

FIG. 50 is a flow diagram illustrative of an embodiment of a routine for determining the prognosis or course of treatment for an individual based on ocular image data.

### DETAILED DESCRIPTION

Managing medical processes such as clinical research, diagnostics, and treatment planning often includes handling large volumes of medical information, including imaging data and test results. Traditionally, these processes rely on a combination of fully automated systems, partially automated workflows, and manual efforts to perform tasks such as annotation, analysis, and data transformation. These tasks are often performed using disparate systems that operate independently, making it difficult to maintain a unified view of the data or track the actions applied to it. This fragmentation can lead to inefficiencies and a lack of traceability, which can be important for facilitating compliance, reproducibility, and data integrity.

To address these or other challenges, some inventive concepts described relate to a robust ontology that defines relationships between data entities, modalities, annotations, and results. The ontology organizes these entities hierarchically, allowing for the structured capture of data across medical workflows, including full traceability from data ingestion to final analysis. This structured framework can allow for seamless integration of both structured and unstructured data, improving reproducibility across trials and diagnostic operations.

Some inventive concepts described herein can improve the management of medical processes by providing a configurable system that allows users to tailor their medical data processing operations based on their specific needs. The system can allow the configuration of processing operations through user-defined criteria, such as through the selection of predefined operations, adjustments to parameters, or the creation of custom processing steps. This flexibility can allow users to adapt the system to their specific workflow requirements, facilitating a coordinated flow of processes. In some cases, these configurations can be facilitated through a low-code or no-code interface, providing users with the ability to create complex workflows without requiring extensive programming skills.

In some cases, the system disclosed herein supports federated data management, allowing for data to be managed across multiple locations or instances within a single unified framework. This flexibility supports multi-site clinical trials and research environments, facilitating secure management of data while remaining accessible to authorized users. Some inventive concepts described herein can provide full traceability for every action, transformation, or annotation performed on medical data. By generating a detailed record for each task, including a unique identifier and details of the operation, the system can facilitate full documentation and auditing. This capability can support compliance with regulatory standards and enable reproducibility, making it possible to trace each modification back to its corresponding step in the process.

Some inventive concepts described herein relate to the integration of human and machine-driven processes. Manual expert input, such as annotation or review, can often operate separately from automated systems, leading to potential data loss or inconsistencies. By integrating human-guided, machine-assisted, and fully automated tasks into a unified processing operation, the system can reduce these risks and improve overall efficiency. For example, human operators can handle complex image interpretations while automated systems manage repetitive tasks, facilitating a smoother and more coordinated process.

The protection of personally identifiable information (PII) and protected health information (PHI) is a significant consideration in medical data processing workflows. Maintaining compliance with data protection regulations while ensuring data traceability is important, especially in complex workflows involving multiple stakeholders. The system can address these challenges by implementing privacy protocols that ensure the secure handling and management of sensitive data throughout the workflow.

The protection of personally identifiable information (PII) and protected health information (PHI) is a significant consideration in medical data processing workflows. Maintaining compliance with data protection regulations while ensuring data traceability is important, especially in complex workflows involving multiple stakeholders. Some inventive concepts described herein relate to addressing these challenges by implementing privacy protocols that ensure the secure handling and management of sensitive data throughout the workflow.

The management of medical images and data in the context of research and clinical trials is notoriously difficult even as the opportunities and demand for imaging biomarkers and artificial intelligence clinical decision support systems rapidly expand. Meeting the demands of image-driven innovation and clinical care in the era of big data and artificial intelligence (AI) generally requires a comprehensive approach that covers the entire medical imaging domain, from hardware definition to observation records, from subject to image, and from anatomy to disease. This approach can be supported by methods to store records and images, transfer data from devices to storage and applications, and curate, visualize, and annotate images. Ensuring the provenance of images and data through algorithm development and validation, as well as protecting individual patient data rights, can be important for maintaining ethical and legal standards in the industry.

While Electronic Data Capture (EDC) systems facilitate the collection and recordation of structured data for clinical trials, they tend to focus on structured data and may not adequately address the collection and recordation of unstructured data, such as medical images. This can lead to the separation of structured and unstructured data, making correlation between them more difficult. In current practice, images and related data are often stored in cloud-based document systems like OneDrive, Box, or Dropbox, which can lead to disorganization and inefficiencies in analysis workflows. Some inventive concepts described herein relate to addresses this by integrating both structured and unstructured data into a unified platform that supports seamless data analysis.

Some inventive concepts described herein address these or other challenges by facilitating the management of complex imaging workflows through advanced data management and workflow automation systems, such as ocuVault™ and ocuTrack™, which are designed to handle multifaceted data across multiple locations. These systems can integrate records, images, functional test data, and metadata from various devices, allowing for batch processing of images, computational analysis, and enhanced role-based access through web interfaces. This provides a flexible, federated data management system that supports compliance, privacy protocols, and the traceability of images and data throughout the workflow.

Analysis workflows in clinical research and clinical trials can include multiple stakeholders, each of whom may have different roles and access rights to PII and PHI. Data coordinators often spend significant time validating, cleaning, deidentifying, and distributing data to appropriate stakeholders, followed by coordinating the retrieval, collation, and review of processed data. These activities are often manual, time-consuming, and prone to error. Some inventive concepts described herein can automate these processes to reduce manual effort and ensure more consistent data handling across the workflow.

Retrospective evaluation of medical images and data is frequently required to validate prior results, uncover new insights, or demonstrate reproducibility. Retrospective evaluation may require sharing data with a collaborator or with an independent third party. Some inventive concepts described herein address the inventory and persistent storage of sets of images and data from directly within the advanced data management and workflow automation system. The persistent storage function allow a user or an automation to bind sets of data at one or more steps in a data processing workflow into an organized electronic binder, assign a permanent or semi-permanent electronic address to the binder, store the binder in an electronic storage facility, and register the electronic address with a registry service.

Another significant cost in data analysis workflows is the masking and randomization of data passed to expert graders for manual annotation and adjudication of images. This process, while critical for ensuring unbiased results, can be labor-intensive and expensive. Some inventive concepts described herein address these issues by automating parts of the masking and randomization process to improve efficiency.

Some inventive concepts described herein relate to the configurability of medical data processing operations. The system can receive input defining specific requirements and adjust processing tasks accordingly, allowing it to adapt to various types of medical data and workflows. Whether processing predefined operations, adjusting parameters based on input, or creating new processes, the system can support a wide range of configurations while facilitating traceability of all actions performed.

Some inventive concepts described herein relate to addressing these or other problems by offering flexible configurability, full traceability, and the integration of human-driven, machine-assisted, and fully automated processes. These systems can improve the accuracy and efficiency of medical data analysis, facilitating well-documented actions and a verifiable processing path. This can enable more effective management of medical data in clinical research, diagnostics, and treatment planning.

Some inventive concepts described herein relate to the creation of human-machine hybrid workflows, where manual expert input (e.g., reviewing images, annotations) is integrated with automated processes. This integration ensures that both human insight and computational power contribute to the workflow's efficiency and accuracy.

As used herein, “ontology” can refer to a structured framework that defines a set of concepts, entities, and relationships within a specific domain. In the present inventive concept, the ontology can refer to a hierarchical model that organizes and captures the data related to medical processes, such as clinical research, diagnostics, or treatment planning. This hierarchical organization defines the relationships between data points, such as the association between subjects, imaging modalities, and diagnostic outcomes. At the top level, the ontology categorizes data based on key entities, such as the circumstances under which data is captured, the medical equipment used, and the associated metadata or annotations. The ontology can be extended to include algorithmic workflow traceability, role-based access management, and integration with application programming interfaces (API) to support automated data handling, as outlined in, for example, U.S. Pat. Pub. 2021/019329, filed Apr. 3, 2020, entitled “Methods, Systems and Computer Program Products for Retrospective Data Mining,” and U.S. Pat. Pub. No. 2021/0209758, filed Jan. 6, 2021, entitled “Methods, Systems and Computer Program Products for Classifying Image Data for Future Mining and Training;” and U.S. Pat. Pub. No. 2023/0023922, filed Jul. 21, 2022, entitled “Methods, Systems and Computer Program Products for Handling Data Records Using an Application Programming Interface (API) and Directory Management System,” the disclosure of each of which is hereby incorporated herein by reference in its entirety.

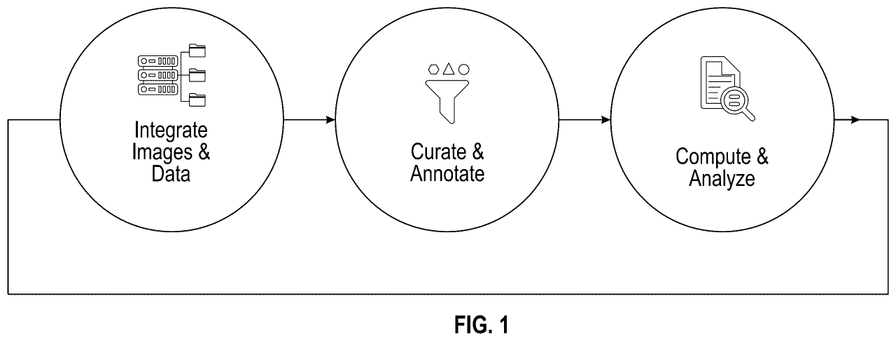

FIG. 1 illustrates an example workflow in accordance with some embodiments of the inventive concepts, showing stages for ingesting images and data, curating and annotating images and data, and performing computations. The workflow can include the following stages: (a) ingesting various types of images and related data; (b) curating and annotating the ingested data; and (c) performing analysis and computations on the ingested images and related data.

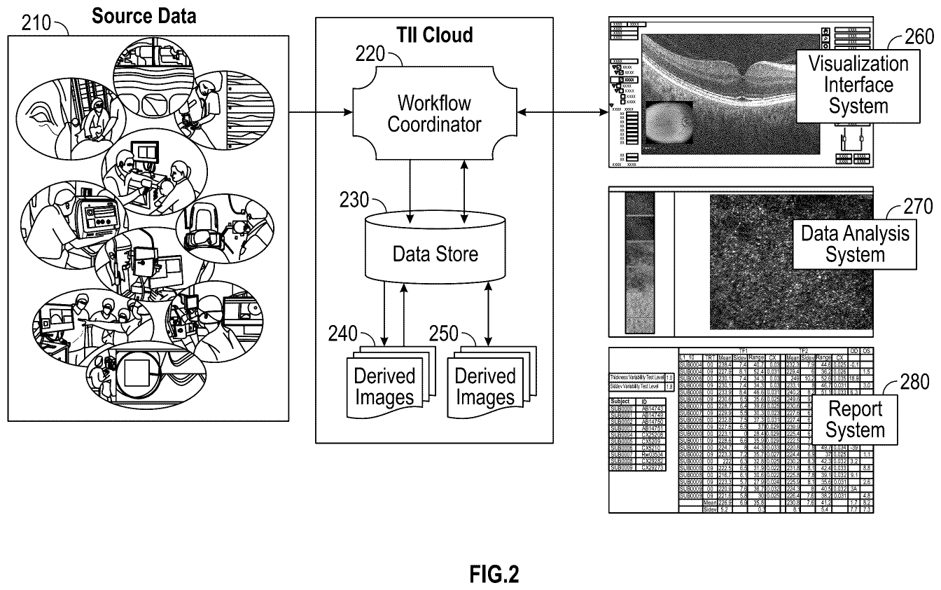

FIG. 2 illustrates an example workflow environment 200 configured to manage and process medical imaging data. The environment includes data intake system 210, a workflow coordinator 220, a data store system 230, source images 240, derived images 250, a visualization interface system 260, a data analysis system 270, and a reporting system 280. The workflow environment can manage medical images and related data collected from various devices and locations, supporting numerous stages of data processing and management.

The data intake system 210 can include, but is not limited to, raw or processed images, or associated medical information collected from imaging devices, clinical equipment, or other data acquisition sources used in diagnostics, treatment planning, and clinical research. Examples of the data intake system 210 can include retinal images, functional test results, and corresponding metadata. In some cases, the data intake system 210 can be composed of structured and/or unstructured information, such as clinical notes, raw image files, or associated patient metadata.

The workflow coordinator 220 can manage the movement and interaction of data between different stages of the workflow environment 200. The workflow coordinator 220 can facilitate tasks such as data ingestion, image curation, and role-based access, ensuring that various authorized users interact with data based on their designated permissions. In some cases, the workflow coordinator 220 can manage the ingestion of source images 240, which are uploaded from external devices, and the generation of derived images 250, which can include processed or computationally modified images.

In some embodiments, the workflow coordinator 220 manages sets of data objects as Packages. Packages may be any combination of structured and unstructured objects. Package contents may be constrained by rules or may be unconstrained. The contents of a Package are the Package inventory. Each Package receives a Universally Unique Identifier (UUID) and the inventory objects receive their own UUID, such that Package and objects each maintain traceability.

The workflow coordinator 220 can also facilitate the transfer of source images 240 and derived images 250 between different systems, such as the visualization interface system 260 for annotation or review, and the data analysis system 270 for further computational processing and analysis. Data is typically stored in the data store 230, where both the source images 240 and derived images 250 are cataloged and managed.

In some configurations, the workflow coordinator 220 can schedule automated processes, such as batch submission and validation of imaging Packages, managing tasks across human and robotic systems, or binding Packages of images and data for persistent storage. These tasks can include image grading, computational analysis, or report generation, which may be performed by integrating the data analysis system 270 and report system 280.

The workflow coordinator 220 can support federated data management, allowing users to access and manage data across multiple instances of the data store 230 from a single interface. This flexibility can be extended to workflows involving both manual and automated operations, ensuring data integrity and compliance with privacy protocols, including PII and PHI. The workflow coordinator 220 can be referred to as ocuTrack™ when configured for medical imaging environments.

The data store 230 can handle the secure storage, management, and retrieval of multifaceted data, including source images 240 and derived images 250, across various stages of the workflow environment 200. The data store 230 can be configured to store source images 240 directly from imaging devices, such as Optical Coherence Tomography (OCT) scans or fundus photographs, which are ingested from external sources as part of the broader data workflow.

The data store 230 can facilitate the organization and storage of derived images 250, which can include computationally processed images, de-identified datasets, or annotated images that have undergone modifications through downstream processes. These derived images 250 may result from operations conducted by the data analysis system 270, where image segmentation, AI-enhanced analysis, or other computations are performed.

The data store 230 can interact with other workflow systems, including the workflow coordinator 220, to ensure that both source images 240 and derived images 250 are accessible to authorized users based on role-based permissions. In some cases, the data store 230 can be integrated with additional computational modules, such as the visualization interface system 260 or the report system 280, which can retrieve or store images for further analysis or reporting.

The data store 230 can support federated data management, allowing multiple instances of the system to be accessed via a unified interface for enhanced control and oversight. This allows for the synchronization and retrieval of records, metadata, and images across multiple locations, ensuring data integrity and compliance with privacy protocols, including protection for personally identifiable information (PII) and protected health information (PHI).

In some cases, the data store 230 can manage advanced processes, such as batching or validation of imaging Packages, in coordination with the workflow coordinator 220. The data store 230 can be referred to as ocuVault™, such as when configured for medical data management environments.

The visualization interface system 260 can be configured as an image visualization and annotation user-facing platform that facilitates user interaction with images stored within the data store system 230. In some cases, the visualization interface system 260 can provide a user-facing application that allows for interactive engagement with images stored in the data store system 230, such as the source images 240 and derived images 250. The visualization interface system 260 can be used to display and curate both source images 240 and derived images 250, supporting various image and functional test modalities, including, but not limited to, Optical Coherence Tomography (OCT), scanning laser ophthalmoscopy, color fundus photographs, adaptive optics fundus imaging, microperimetery, electroretinography, and other imaging and test data commonly employed in medical or clinical research contexts.

The visualization interface system 260 can include functionalities such as zooming, panning, and rotating images to enable detailed examination, as well as the ability to overlay multiple images to facilitate comparisons across different modalities or temporal changes. The system can be suited for clinicians, researchers, or graders who are tasked with inspecting anatomical features, performing comparative analyses, or tracking disease progression across different timepoints.

In some cases, the visualization interface system 260 can support features that enable the annotation of regions of interest (ROIs) directly onto images. ROIs can refer to specific areas of an image identified for further analysis, grading, or computational processing, and can be useful in applications such as disease diagnosis or anatomical studies. These annotations can be saved within the data store system 230, preserving traceability of all modifications and interactions with the data. Additionally, such annotations can be linked to other datasets or analytical results processed by the data analysis system 270, allowing for comprehensive data management that integrates both visual and computational insights.

The visualization interface system 260 can be interoperable with other modules, including the workflow coordinator 220 and the data analysis system 270, facilitating integrated data flow across various stages of the workflow. The visualization interface system 260 can support the extraction of Regions of Interest (ROIs) from larger image sets. ROIs can refer to specific portions or areas within an image that are selected for detailed examination, grading, or computational analysis. In medical imaging, ROIs can be often defined as areas that include anatomical features, abnormalities, or other significant regions that require closer inspection, such as retinal areas showing signs of disease progression or structural changes.

The visualization interface system 260 can allow users to annotate and select these ROIs, which can then be utilized for further computational analysis or grading by human experts. Once extracted, these ROIs can be processed by the data analysis system 270 or linked to additional datasets. The processed results, along with the extracted ROIs, can be stored back into the system, ensuring that the data remains integrated within the overall workflow. This functionality ensures that both visual and computational analysis can be seamlessly incorporated, preserving data integrity and enhancing the traceability of all modifications throughout the workflow.

In some cases, the visualization interface system 260 can be referred to as ocuLink™, such as when configured for environments focusing on medical image curation and analysis.

The data analysis system 270 can manage the processing and analysis of large sets of images, including original source images 240 and/or modified or derived images 250, for example within clinical or medical imaging workflows. The data analysis system 270 can perform tasks such as, but not limited to, automatically analyzing images, identifying regions of interest (ROIs), or calculating relevant metrics from the images. For example, in ophthalmology research, the data analysis system 270 can analyze retinal images to measure the thickness of retinal layers or cellular spatial statistics at points in time or monitor structural changes over time and may further correlate such structural results with functional test results.

The data analysis system 270 can handle batch processing of images, allowing for efficient automation of workflows. The data analysis system 270 can de-identify images removing personal information), randomize them to reduce bias, and compute results based on these processes. These computational results, such as image metrics or statistical summaries, can be stored in linked databases for further analysis or reporting. The data analysis system 270 can integrate these results with other datasets to provide comprehensive insights for clinical or research purposes.

The data analysis system 270 can be fully automated or at least partially user-directed, depending on the requirements of the specific workflow. The data analysis system 270 can automatically grade images, compare them to standard reference images, and manage the flow of data through multiple analysis stages, ensuring that all operations remain traceable and compliant with protocols.

The data analysis system may include a plug-in architecture with an Application Programming Interface, supported by a Software Development Kit, that allows a user to integrate third party software modules, where the API and SDK support mapping data inputs from the workflow system to the software module and outputs from the software module to the workflow system, thereby maintaining flexibility while sustaining traceability throughout the workflow.

In some cases, the data analysis system 270 can be referred to as ocuLytics™, such as when it is used for batch processing and performing complex image calculations in medical imaging environments.

The reporting system 280 can generate structured outputs derived from the data processed by the data analysis system 270, providing clear and organized summaries of results. The reporting system 280 can produce statistical analyses, visual representations of data, or annotated images, which may be used for various purposes such as clinical evaluations, research studies, or regulatory compliance.

The reporting system 280 can be configured to allow users to customize the format, structure, or content of the reports based on specific needs. For example, reports can be tailored to meet the distinct requirements of different research protocols, clinical workflows, or regulatory submissions. These reports can include detailed breakdowns of the images and data, ensuring that the information is presented in a format that is both comprehensive and user-friendly.

In some cases, the reporting system 280 can incorporate audit logs, tracking each step of the data modification process for both the source images 240 and derived images 250. This functionality can facilitate full traceability, providing a detailed history of the actions performed on the data throughout the workflow, which can be important for meeting compliance standards and ensuring reproducibility.

The reporting system 280 can be integrated with other systems in the workflow, such as the workflow coordinator 220, to automatically generate reports at predefined stages of the process, reducing the need for manual intervention and enhancing the efficiency of the overall workflow.

The workflow environment 200, as depicted in FIG. 2, can include the ingestion of images, functional test data, and metadata from multiple devices and locations into a centralized cloud repository. These records, which can include source images, related objects, and derived images, can be stored within the data store system 230. The data store system 230 can be configured as a model-based hybrid database system that manages multiple interoperable databases. Derived objects may include de-identified and masked copies of images or computationally modified versions of the original data, enhancing data privacy and supporting further analysis.

The workflow coordinator 220 can act as a platform for coordinating user interactions with the data store system 230, allowing for the management and control of records and objects stored within the system. The workflow coordinator 220 can provide a web interface to facilitate the execution of workflow operations, such as task assignments and data management processes. Additionally, the visualization interface system 260 can provide tools for image visualization, curation, and annotation of the records stored in the data store system 230. This system can include interoperable databases that manage enriched data linked to the annotations made during the workflow. The data analysis system 270 can handle batch processing of images and derived images, computing metrics from these datasets. Results from the analysis can be stored in linked databases for statistical analysis and reporting, with classification and storage across one or more additional databases where needed.

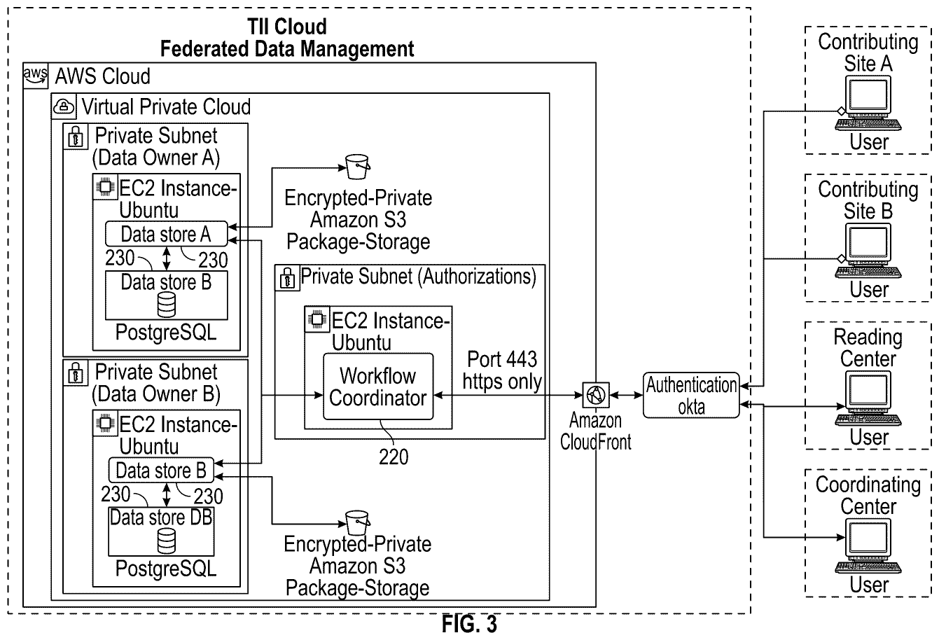

FIG. 3 illustrates an example cloud-based architecture for data and workflow management in accordance with some embodiments of the inventive concepts. Users with unique roles access the workflow coordinator 220 through an authentication engine. Roles may include data contributors, image graders, or project coordinators. Authentication can be required, and role assignments may dictate access to data and the actions that users can perform on such data.

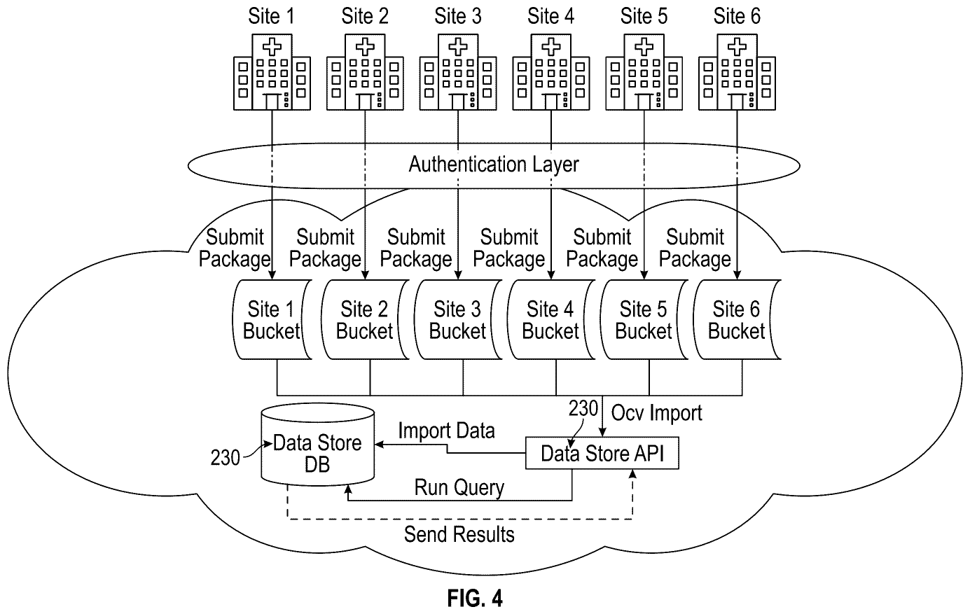

FIG. 4 illustrates a hierarchical organization of entities for managing data related to medical research, clinical trials, or clinical evaluations. In some instances, multiple instances of the data store system 210 can be accessed through a common interface in the workflow coordinator 220, enabling federated data management. Teams may be structured to either be unique to one instance of the data store system 210 or bridge multiple instances. Process automation features allow for leveraging data from multiple instances of the data store system 210 while protecting privacy and proprietary information.

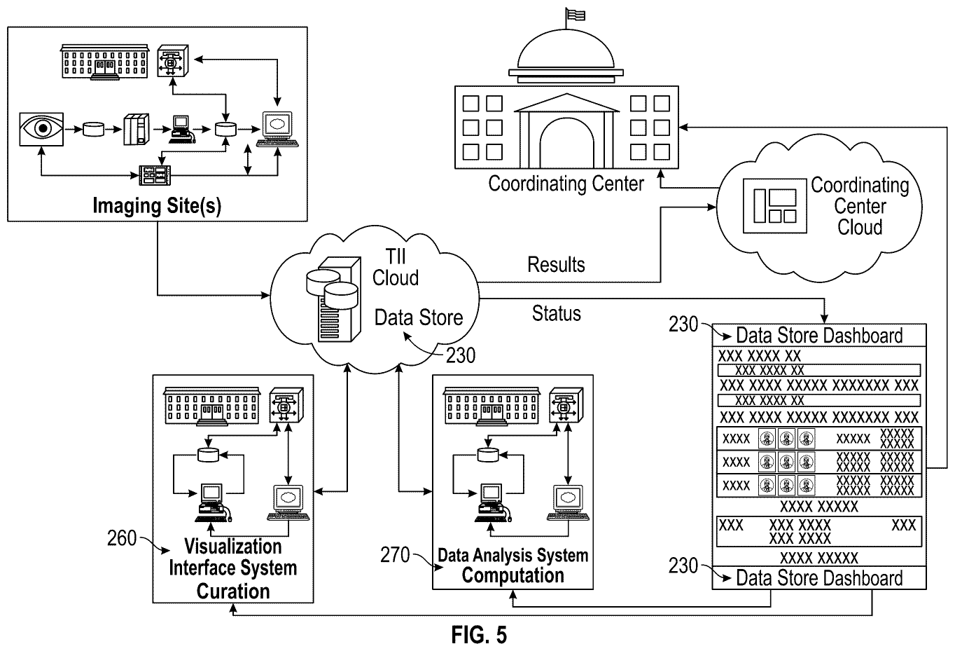

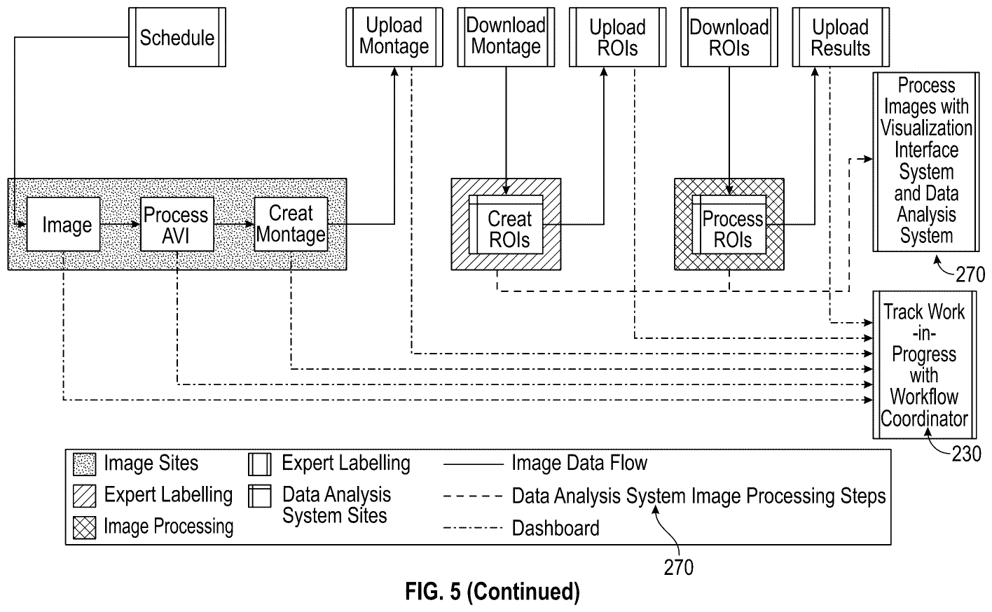

FIG. 5 illustrates an example workflow for the analysis of retinal images in a clinical research or trial environment. Retinal images are acquired at imaging sites according to a specific study protocol, and subsequently uploaded to the data store system 210 for storage. Multiple discrete analysis steps pull images from the cloud for local analysis and upload results back into the data store system 210. A coordinating center retains access to data and results, as defined by the study protocol, with transparency and traceability supported through a dashboard that tracks progress for various stakeholders.

The specific embodiment of FIG. 5 relates to a complex workflow for obtaining high-resolution images of the retina, which are then analyzed for rod and cone photoreceptor structure. Processing steps may be initiated locally at imaging sites, with data combined to form a montage uploaded via the workflow coordinator 220. Experts can download the montage, select regions of interest (ROIs), grade the regions, and upload the results through the workflow coordinator 220. These graded ROIs can then be used for computational analysis and uploaded back to the cloud through the workflow coordinator 220.

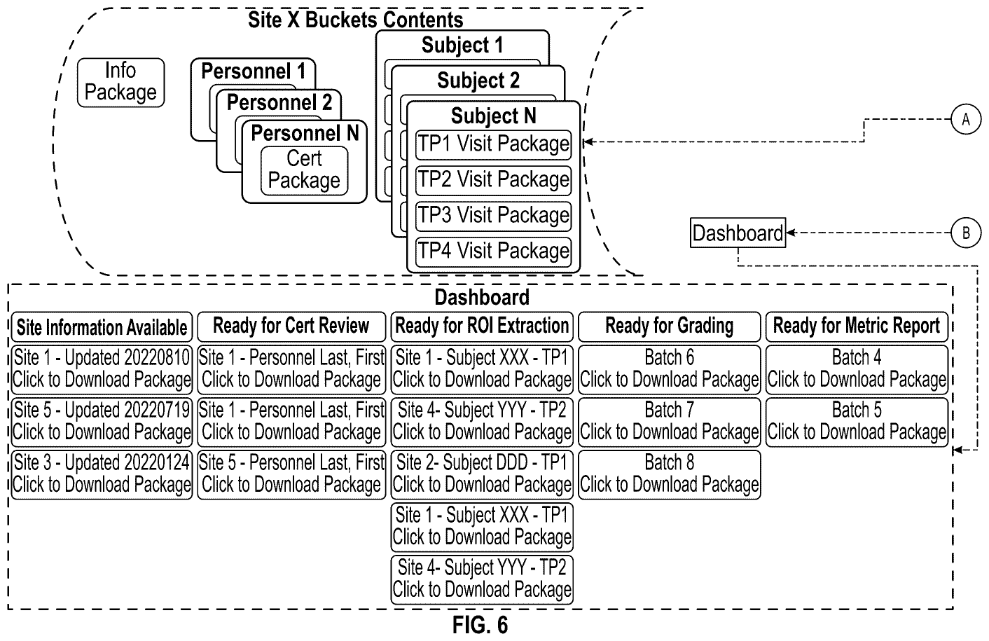

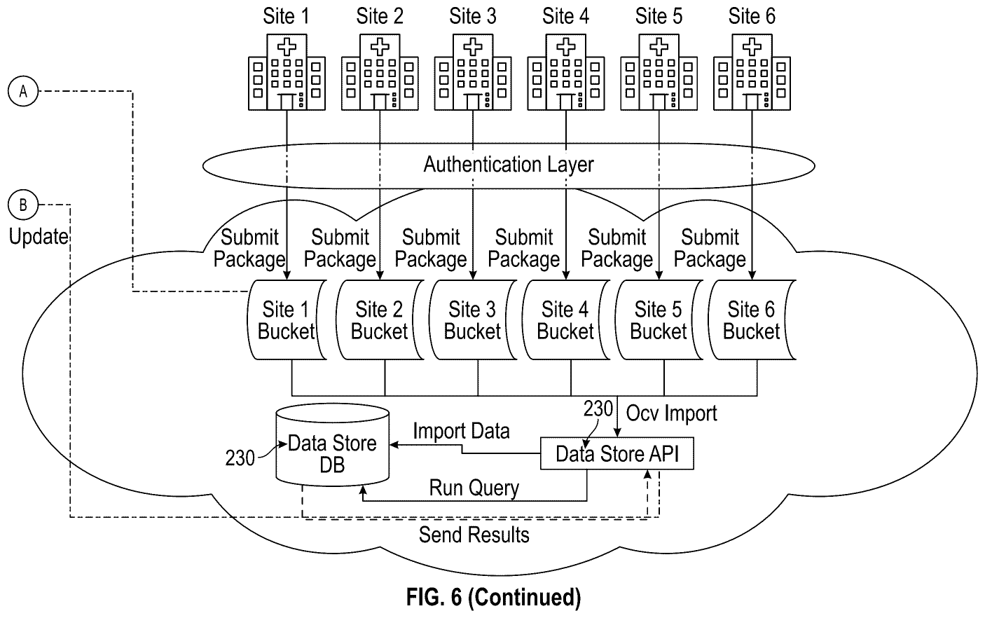

FIG. 6 illustrates an example dashboard interface for tracking site information, personnel certifications, and stages of image analysis. Such dashboards can be used for monitoring and tracking image analysis workflows specific to retina image studies.

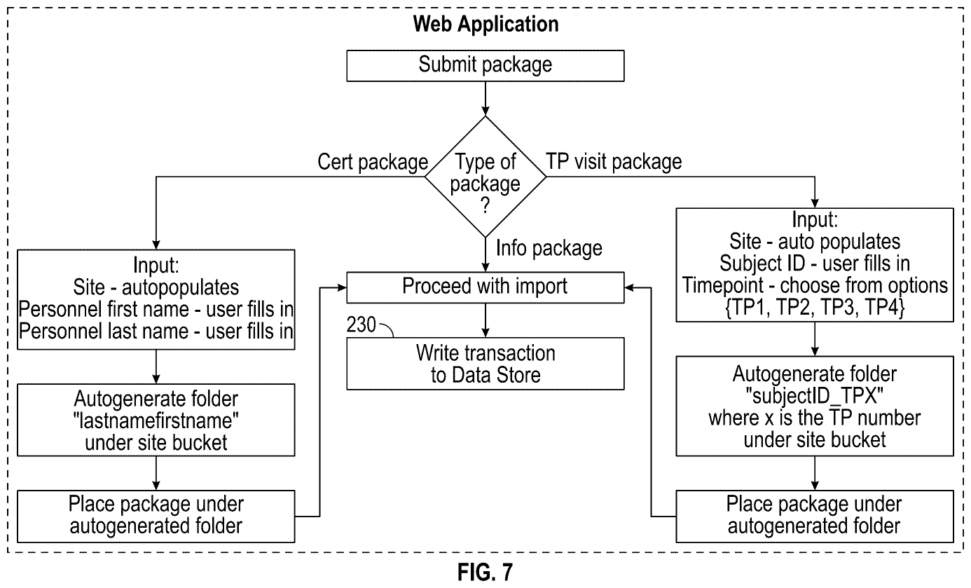

FIG. 7 illustrates an example user interface for selecting a Package type and submitting it with predefined rules and content requirements. Users may select a Package type, and the workflow coordinator 220 provides specific instructions on how to submit the Package content. Validation checks may be performed prior to upload, and once validated, the Package can be uploaded to the data store system 210 and stored in an autogenerated location.

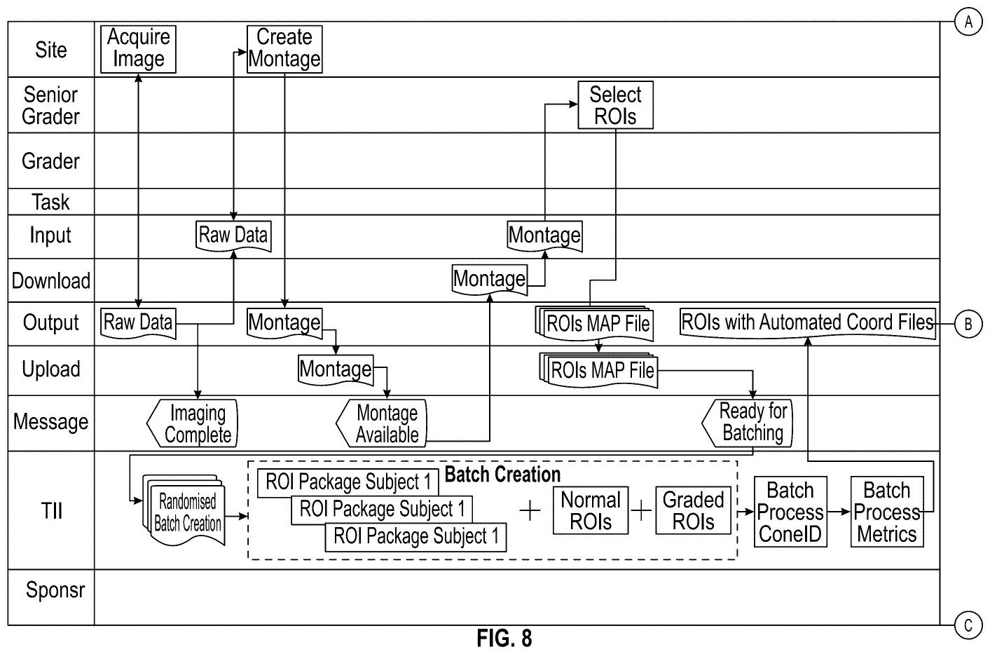

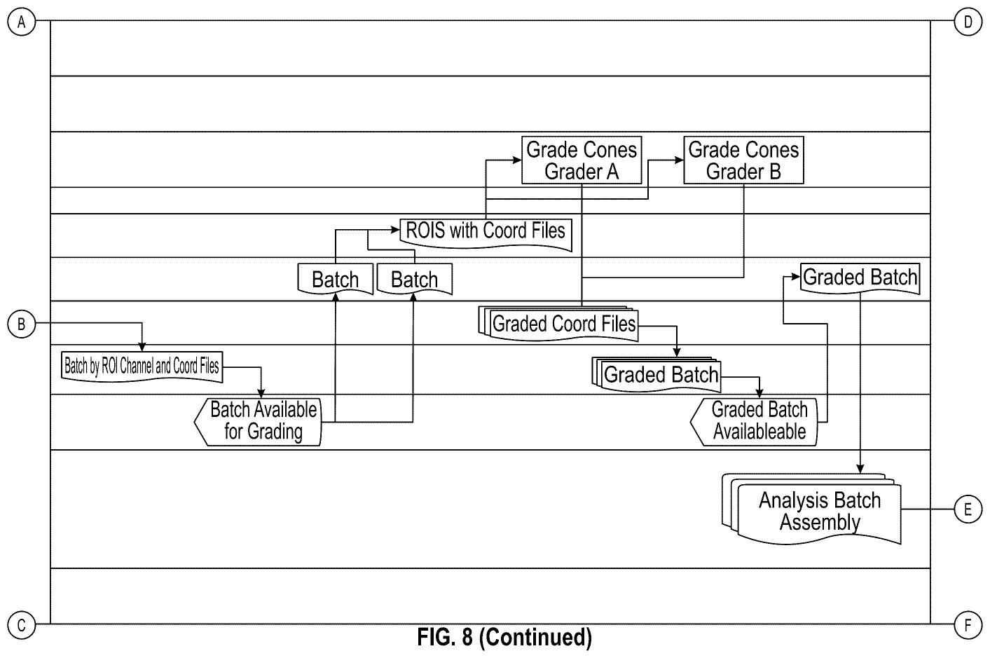

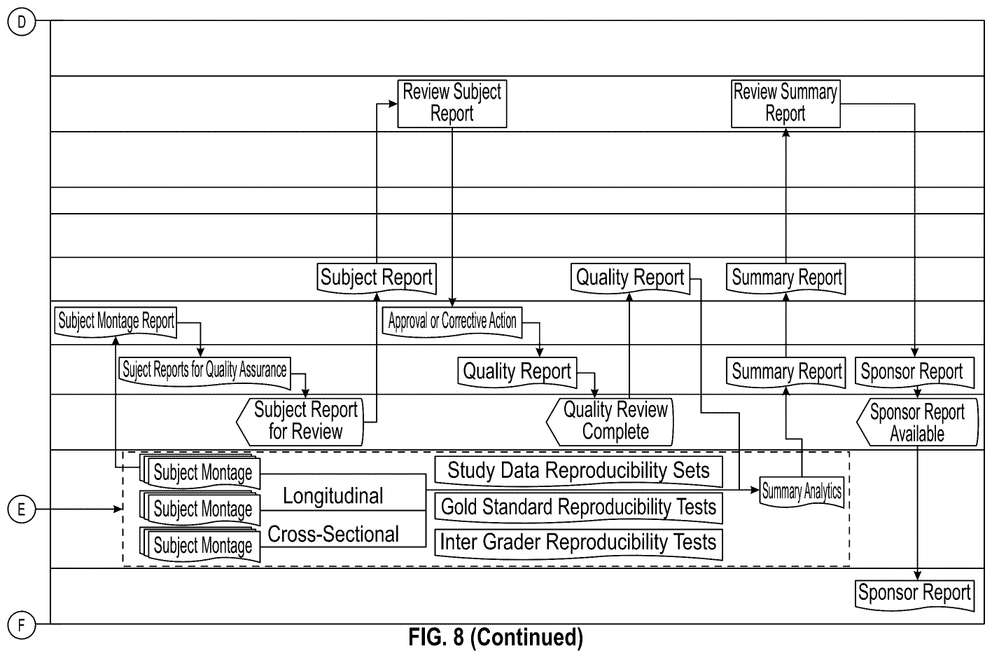

FIG. 8 illustrates an example real-world image processing workflow according to some embodiments of the inventive concepts. This workflow can include a series of deterministic, ordered steps planned and executed, similar to the pattern of a musical score. Inputs, actors, and outputs can be defined, with a configurable model provided for each step of the workflow. Methods for transporting images and data through these steps, and for recording the objects, attributes, and actions related to each step, are defined in the system.

It is a feature of the present inventive concept to define the inputs, methods, actors, and outputs for a sequence of image and/or data analysis steps, define a configurable model for each step, define an ordered set of operations of the steps, provide methods for transporting images and data through the ordered set of operations, provide a database and data model for recording the objects, methods, values, attributes, and actors for the steps throughout the processing workflow. The various operations may require human interaction, may be autonomous robotic data operations, or may be hybrid operations that integrate human and robotic data operations for any process step. The data may be source data, derived data, de-identified data, otherwise masked data, randomized data, computational outputs, statistical outputs, classifications, graphical outputs without limitation, but as defined, constructed, and programmed for the study workflow.

For unbiased analysis, images and test data may need to be masked, randomized, and batched for distribution to certain processing steps. For example, expert human graders may annotate images without any a prior knowledge or clues about the subject during the grading process. In some embodiments of the present inventive concept, robotic operations can be deployed to mask, randomize, batch, and distribute image sets for grading. The masking operations can be defined according to the study protocol. Removal of PII/PHI is frequently required. Other information may be masked or may be visible according to the protocol. For example, patient sex may be masked or disclosed, for example, if proper grading requires sex-dependent anatomical knowledge.

At the same time, the randomized data will need reorganization for longitudinal analysis of subjects over time and cross-sectional analysis of population data at a point in time. The analysis may require association with other images, test data, interventional information, and other metadata, and therefore the original organizational state should be recoverable. In some embodiments of the present inventive concept, cross-sectional statistics and longitudinal statistics are processed automatically as data is accumulated.

Furthermore, it can be important to provide rapid feedback on the quality of analysis. In some embodiments of the present inventive concept, performative statistics are processed automatically as data is accumulated. Three dimensions of reproducibility may be monitored: Inter-grader reproducibility tests dependence on human graders when multiple graders are given the same data to analyze; Study data reproducibility tests reproducibility of analyzing study data that is subject to random re-analysis; and Gold Standard reproducibility tests stability of results on gold standard data that is randomly interlaced with the study data. In some embodiments of the present inventive concept, inter-grader reproducibility is automatically calculated and tracked as data accumulates. Study data that has already been analyzed is randomly selected and folded into new grading batches, and re-test reproducibility is automatically calculated and tracked. Gold standard data is pulled from an existing library, folded into grading batches, and re-test reproducibility of known gold standard data is automatically calculated. The associated variances may be tracked in control charts that are visible to program management and coordinators, and alarm flags initiated when the process is out of control.

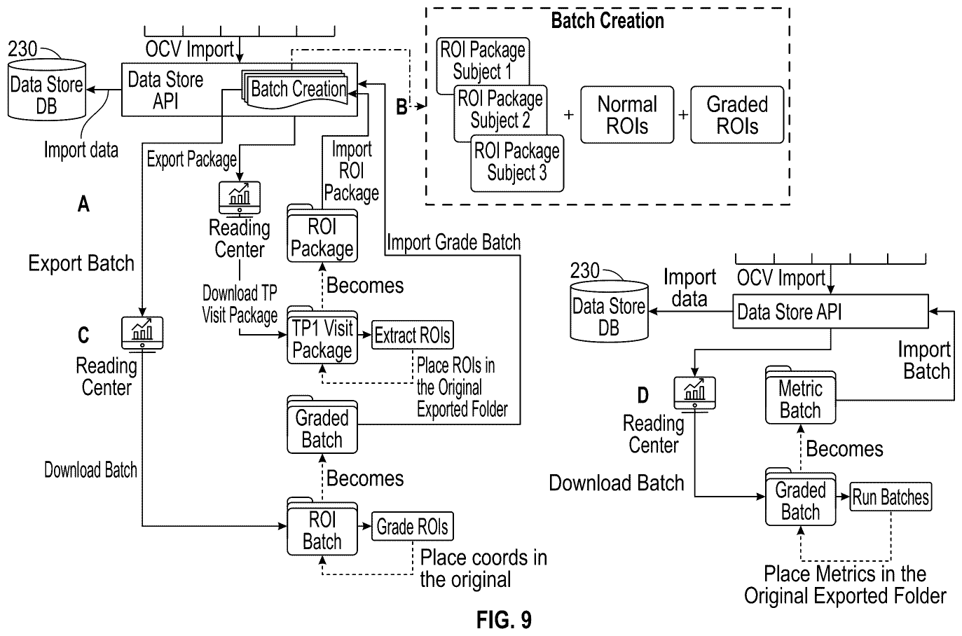

FIG. 9 illustrates an example image processing workflow involving the selection and extraction of regions of interest (ROIs) from image montages derived from adaptive optics fundus images.

After a site has submitted a Package from an imaging session, a first Grader will download a visit Package (A) as prepared by ocuTrack. The Grader extracts relevant Regions of Interest (ROIs) from the visit montage and submits the extracted ROIs in a new ROI Package. By construct, the ROI Package maintains complete traceability to the source Montage as well as to the Grader extracting the ROIs.

In the next step, ocuTrack automatically creates Batches (B) of masked ROIs for the next step in the Grading process. Instructions scripted into ocuTrack specify rules for randomizing ROIs, interleaving Gold Standard ROIs, and interleaving previously graded ROIs for re-test reproducibility testing.

One or more Graders, according to rules scripted into ocuTrack, will then download masked and randomized Batches for ROI Grading (C). The graded ROI batches will again be submitted to ocuTrack, registered to the database, and grading result objects stored with full traceability.

In a further step, results of ROI grading will process to a computational step (D) for computing metrics derived from the graded ROIs. The step (D) shown implies human intervention, though this step may be done robotically in the cloud, robotically at the desktop, or semi-autonomously with direct user intervention. Similarly for step (C), Grading may be done robotically given validated grading algorithms, and humans may be deployed for a quality check. The quality check might be a full review, or a statistically sampled review, according to the reliability of the processes at any point in time.

Robotic process automation (RPA) for creating, distributing, retrieving, and tracking masked grading batches is a particularly valuable improvement to current manual process. Batch management RPA reduces manual effort and errors and provides a level of traceability that cannot be replicated in a manual process.

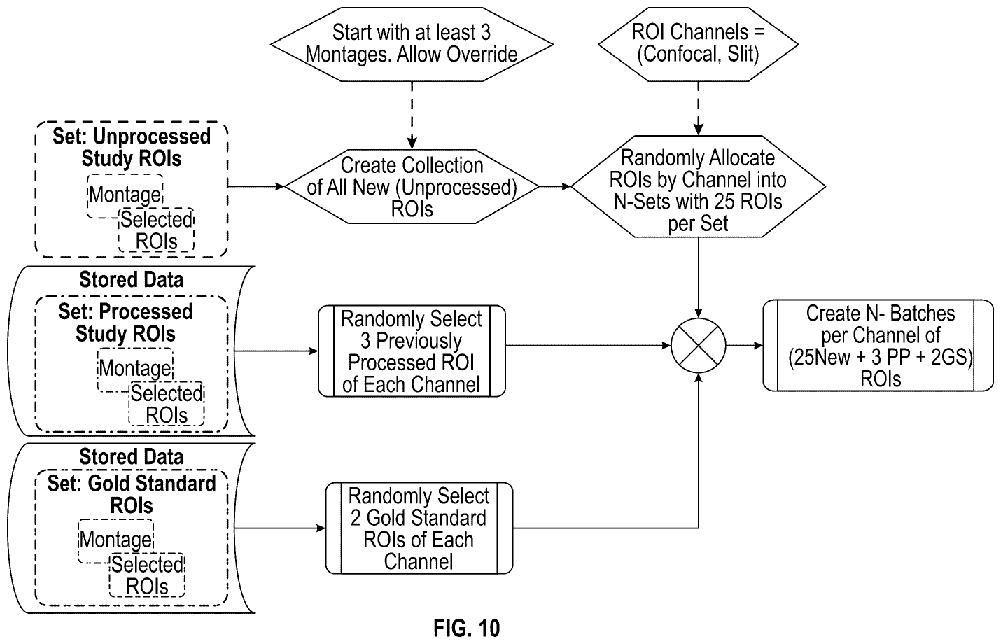

FIG. 10 illustrates the beginning of a robotic batch process automation (RBPA) after the extraction of ROIs from image montages. A rule is established that masked batches of ROIs should be randomly drawn from at least three independent subject montages. Batches may contain randomized ROIs from multiple individuals to prevent bias in human grading.

In FIG. 10, the RBPA begins after the extraction of ROIs from image montages. A rule is established for this example that masked Batches of ROIs should be randomly drawn from at least three independent subject montages. In the retina imaging workflow discussed herein, an exemplary montage may reflect an 8×8-degree field of view of the retina and an individual ROI for analysis may be on the order of 1×1 degree. ROIs are extracted in this circumstance for several reasons: avoiding regions of bad image quality, avoiding regions with vasculature, selecting regions at various landmarks across the retina. The rules are application specific. A consideration here is that the individual image of the subject is decimated into numerous regions of interest for analysis, and a Batch may include deidentified and randomized ROIs from multiple individuals to avoid bias in the human grading step.

Continuing further with FIG. 10, the deck of ROIs from at least three subjects are randomly distributed into N-sets of ROIs. The target is set in the configuration rules according to the batch size that is reasonable for a single Grading session; here, the target nominal batch size is 25 ROIs per grading session. To each of the N-sets of ROIs, a small number of previously graded ROIs are randomly selected and added, and a small number of Gold Standard ROIs are randomly selected and added. The result is a set of N-Batches, each containing approximately 25 new ROIs, 3 previously processed ROIs, and 2 Gold Standard ROIs.

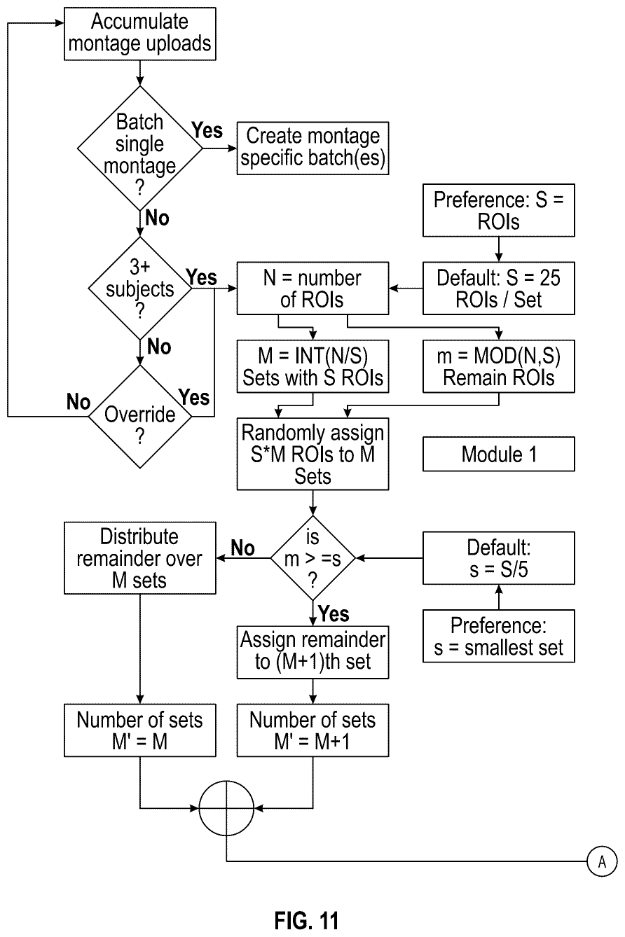

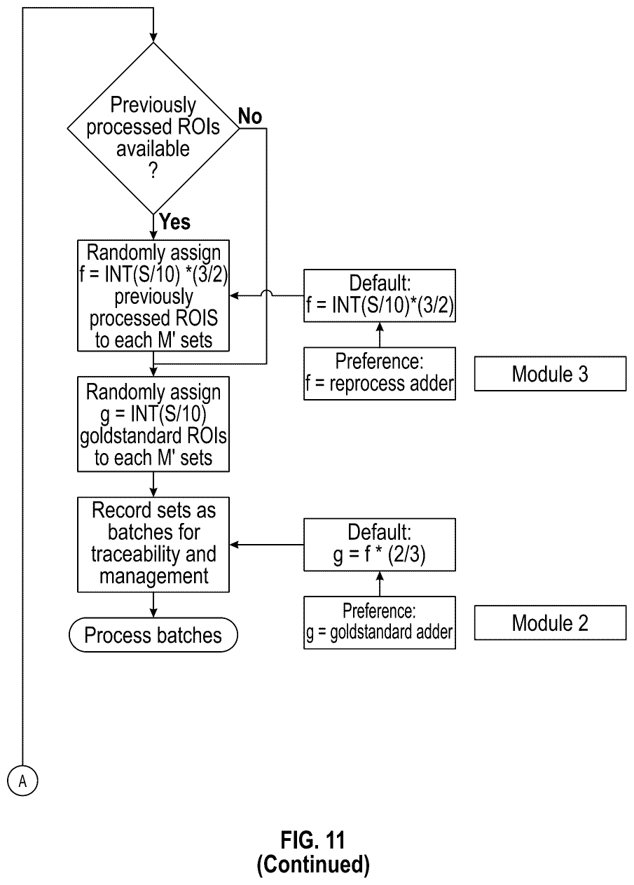

FIG. 11 illustrates a flow chart of an example batch allocation algorithm with user-settable parameters in accordance with some embodiments of the inventive concepts. Parameters such as the number of independent images required for batching and the target batch size can be user-configurable. The flow chart of FIG. 11 shows one Batch allocation algorithm with the following user-settable parameters:
1. Create Batch? Boolean
2. Minimum Number of Independent Images required for Batching. Integer
3. Target number of items to fill a batch. Integer
4. Target number of items to add for reprocess quality testing Single (ratio of int.)
5. Target number of items to add for Gold Standard quality testing Single (ratio of int.)
6. Rule for distributing remainder ROIs (minimum Batch) Integer

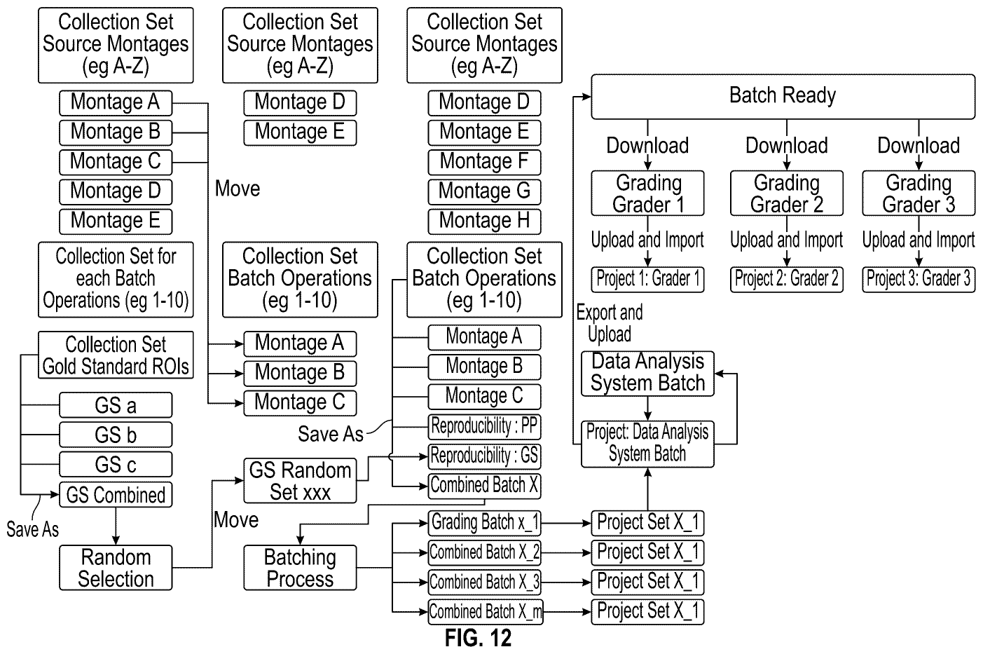

FIG. 12 illustrates a concept of collections and projects extended to support functionality within a workflow in accordance with some embodiments of the inventive concepts. Gradable objects, such as ROIs drawn from source montages, can be assigned to collections within a gradable image collection set. In a further embodiment of the present inventive concept, Batches are recorded as Collections and the processes of Grading are recorded in Projects of the Collections. The concept of Collections and Projects may be extended to Collection Sets and Project Sets for functionality as diagrammed in FIG. 12. Gradable objects (e.g., ROIs drawn from source Montages) may be collectively assigned by source montage to Collections within a Gradable Image Collection Set (GICS). In the RBPA, a subset of Source Montages may be moved to a Batch Operation Collection Set (BOCS).

Gold Standard images may be assigned to a Gold Standard Image Collection Set (GSICS). In the RBPA, a random subset of GS images may be drawn from the GSICS into a new Randomized Collection within the GSICS and moved to Collection (GS) within the BOCS. Similarly, previously analyzed images may be randomly selected and copied to Collection (PP) within the BOCS. The BOCS will then have a multiplicity of Collections ready for batching, e.g.: Collection A-C: Montage ROIs A-C; Collection PP: Previously Processed ROIs; Collection GS: Gold Standard ROIs. As a step in the RBPA, these collections are combined to form the master batch, and the ROIs from the master batch are randomly allocated to Gradable Batches according to the distribution rules. Each Gradable Batch is now a member of its own Collection. The distribution rules for allocating PP and GS images may be distributed ON AVERAGE to each separate gradable batch, or they may be distributed so that the allocation rules are met for each individual gradable batch of the set.

Grading proceeds in a series of Projects unique to each Gradable Batch Collection. Automated grading may be applied to a first Project and Graders may be asked to correct the automated grading. In this case, the Automated Grading Project May be copied for distribution to an arbitrary number of Graders for correction. Alternatively, Graders may be asked to perform zero-based grading. Performance of Graders may be evaluated against each other and against the autonomous grading. Similarly, multiple autonomous grading algorithms may be pitted against each other and against human Graders, and corrective grading and zero-based grading can be deployed in parallel. The system of Collections and Projects maintains tremendous flexibility with inherent traceability. Note that autonomous grading may be programmed to run without human intervention in the cloud, or a user may be instructed to access a project batch through ocuTrack for local computation. Human graders will access the batches through ocuTrack, grade, grade locally, and resubmit results through ocuTrack. Or ocuTrack may invoke a web-based grading application such that batches are never moved from the cloud environment.

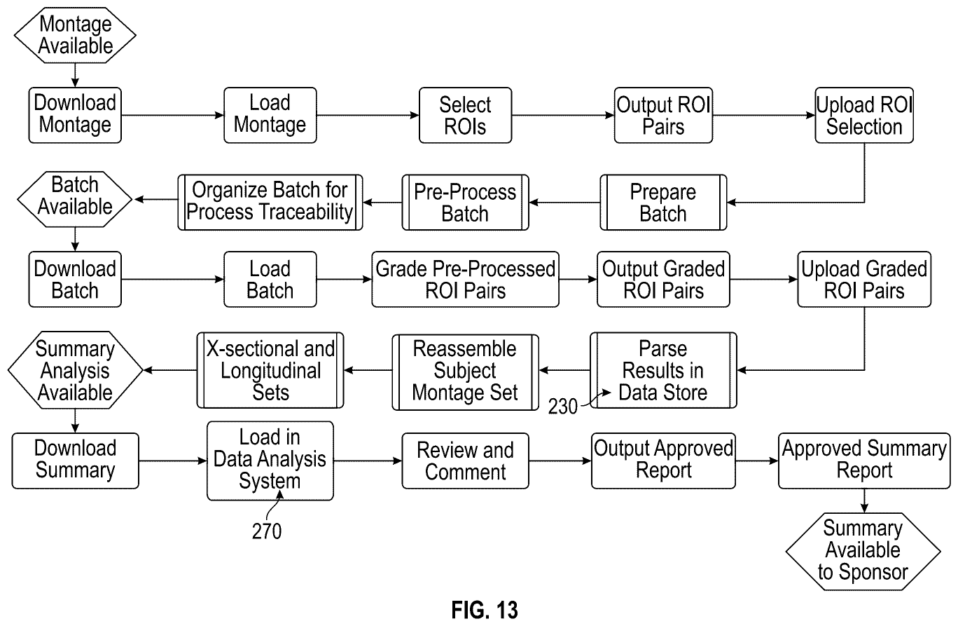

FIG. 13 illustrates a linearized process flow reflective of an example workflow embodiment in accordance with some embodiments of the inventive concepts. The process may include the analysis of complementary ROI pairs for multi-modality analysis.

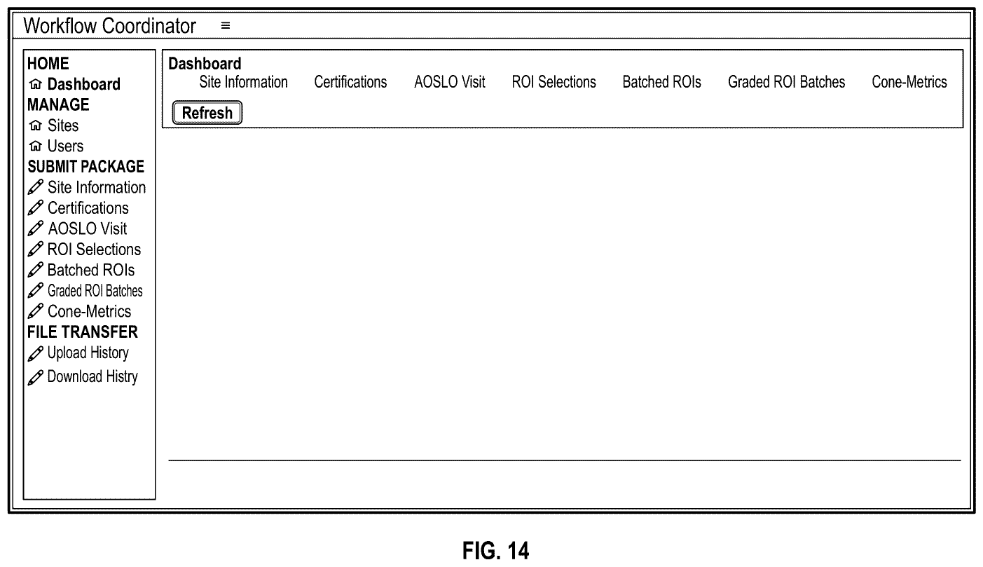

FIGS. 14-22 provide user interface displays of the ocuTrack software program product as an embodiment of the present inventive concept. FIG. 14 is a display of a main screen of the UI. The left had column is a menu of available actions. HOME navigates to a Dashboard display for the state of the workflow. MANAGE navigates to administrative pages for establishing Sites and Users. SUBMIT PACKAGE navigates to the pages for the submittal of content. FILE TRANSFER navigates to records of upload and download histories.

FIG. 14 illustrates an example display of the main user interface screen for a workflow management system, showing a menu of available actions such as dashboard access and Package submission.

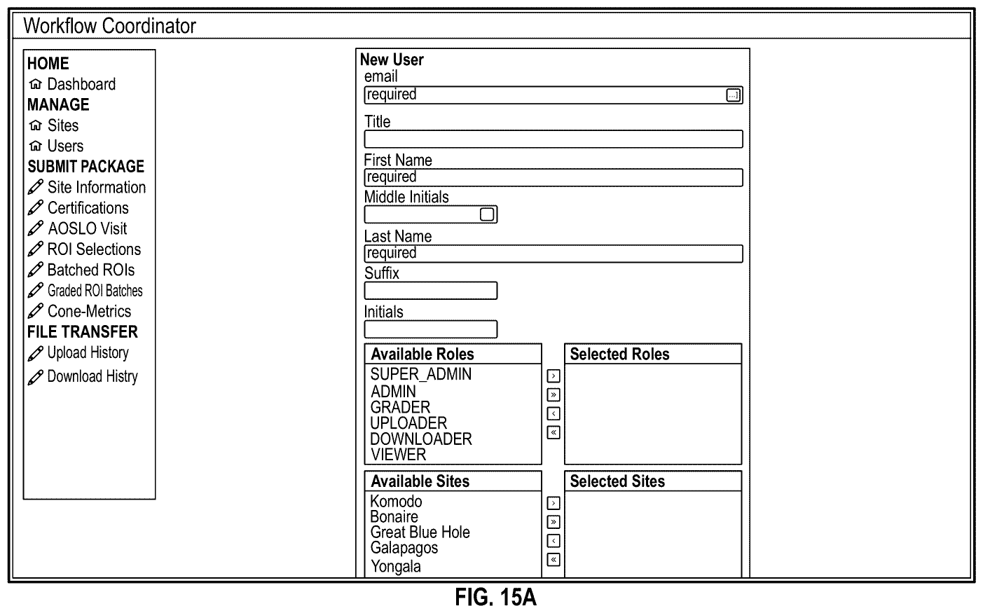

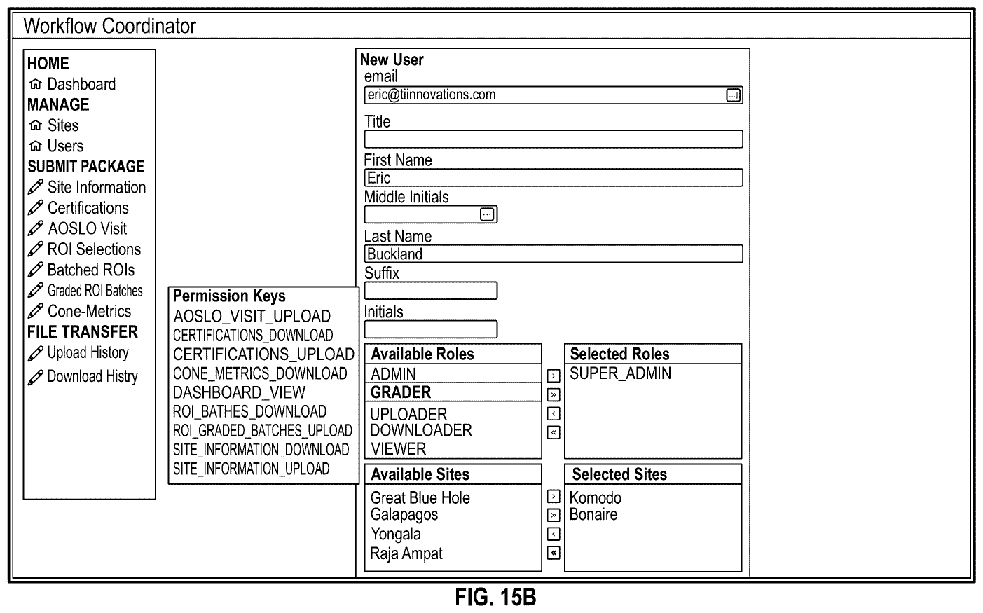

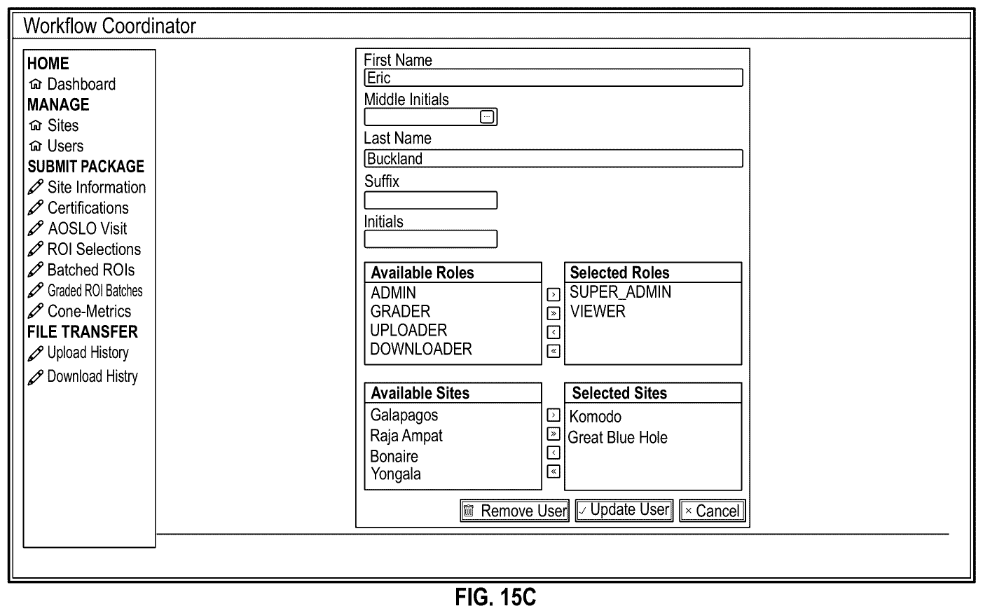

FIGS. 15A-15C illustrate example administrative pages for adding new users, including role assignment and site access configuration. FIG. 15 shows three states of the administrative page for adding new Users. The top screen (A) shows field for User information and assignment to Roles and Sites. A group of one or more Sites forms a Team in this embodiment. Sites define the scope of data access for a User. User Roles define the actions that a User may take on accessible data. A set of User Roles with a tooltip showing the Permission Keys associated with allowed actions for the Grader role is highlighted in screenshot (B). The assignment of a User to multiple Roles with access permissions to data from multiple Sites is highlighted in screenshot (C).

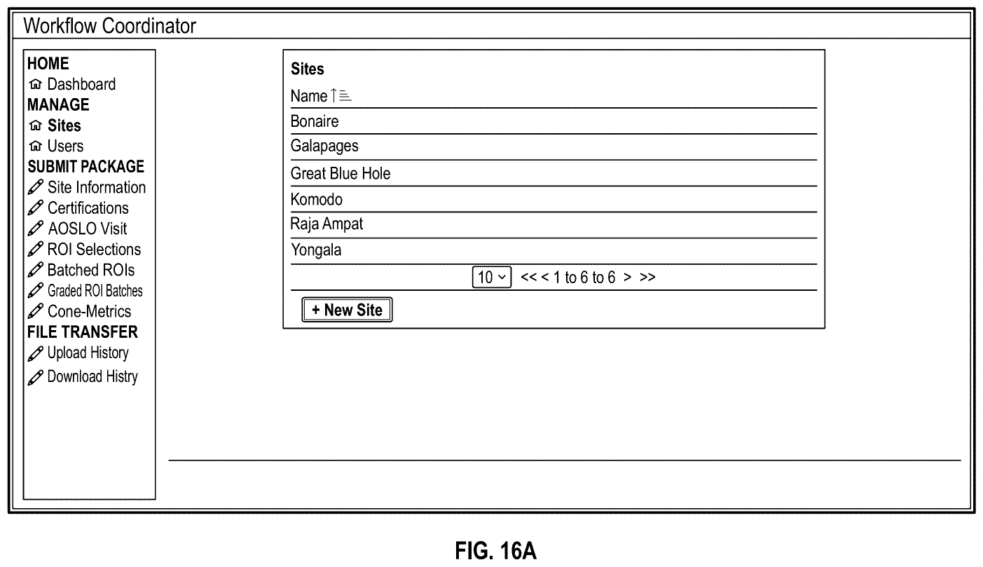

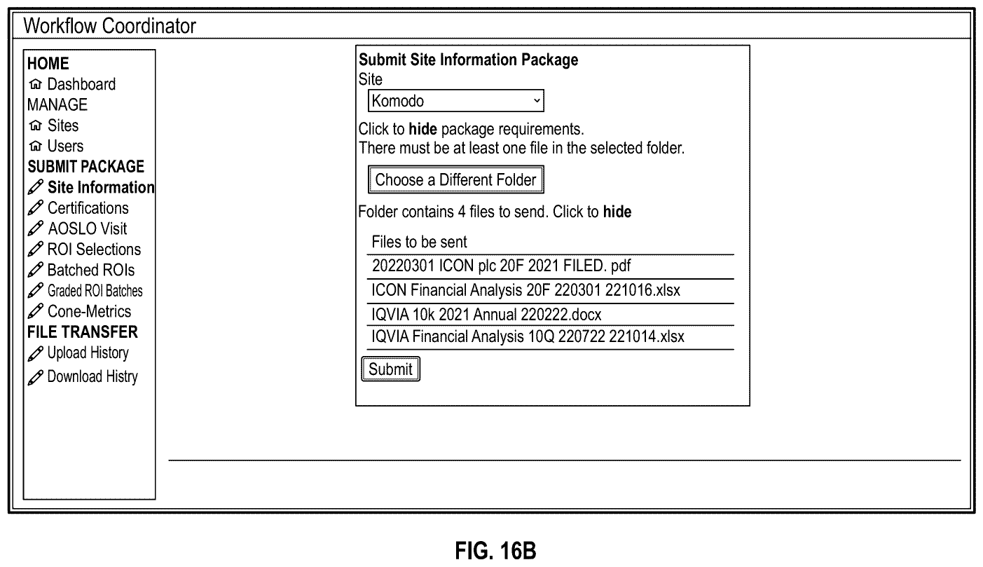

FIGS. 16A and 16B illustrate screenshots of participating sites and an example interface for submitting site information, respectively. FIG. 16(A) is a screenshot of participating Sites. FIG. 16(B) is a screenshot of the interface for submitting Site Information. In some embodiments of the present inventive concept, the User selects a Site from a dropdown menu of Sites to which the User is a member. Validation Requirements for Site Information Package are displayed to the User. The User browses to a folder with the Site Information to be uploaded. ocuTrack validates that the selected folder meets the Validation Requirements locally, prior to uploading. ocuTrack displays the contents to be uploaded and any error messages, providing the User the opportunity to take corrective action prior to initiating any slow or costly internet transfer.

In some embodiments of the present inventive concept, the Validation Requirements are set forth for Packages at each stage of the workflow. Validation Requirements may be a minimum set of requirements, allowing the User to submit content exceeding the minimum requirements. Validation Requirements may also be a complete set of requirements, constraining the user to submit only the data that is required for the workflow stage. In the former case, a User may wish to submit supporting documentation, photographs, pictures of handwritten notes, voice memos, and the like without folder locations, file naming conventions, and the like. All objects register to the ocuVault database and store with the required objects for rapid recovery. In the latter case, firm validation requirements eliminate the risk of sending data that is inappropriate to the process.

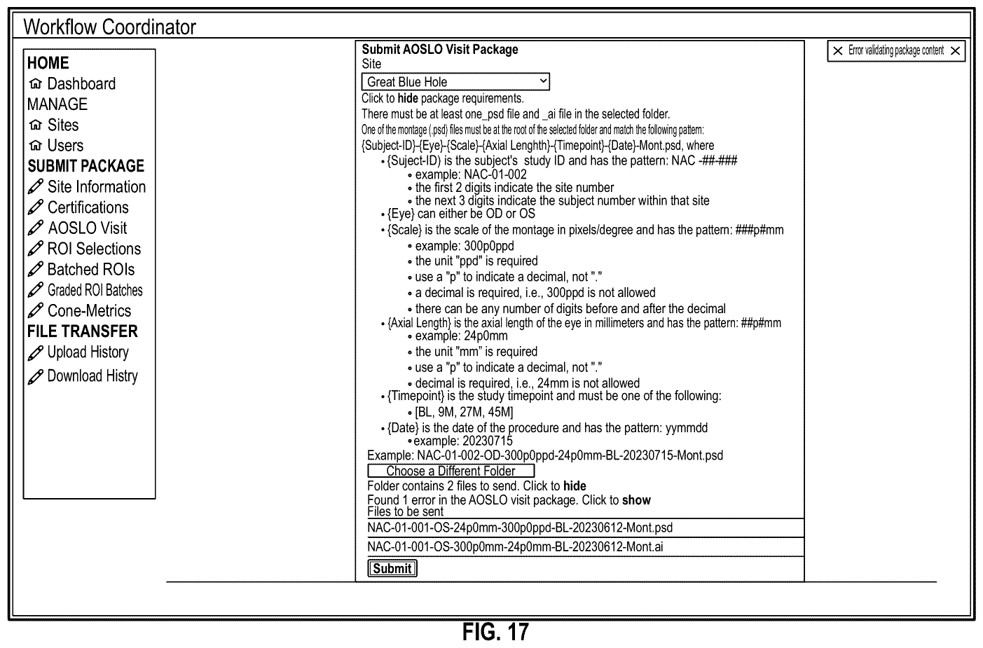

FIG. 17 illustrates an example user interface screenshot for submitting an imaging visit Package in the workflow system. FIG. 17 shows a UI screenshot for an Imaging Visit Package submittal. The User selects the originating Site from among authorized Sites. Detailed validation rules for the imaging Package are displayed, validation is performed prior to upload, and contents and errors are displayed to the User for any necessary corrective action. In some embodiments of the present inventive concept, this imaging Package requirements include a specific file naming convention to facilitate compliance to the study protocol, provide metadata necessary for proper database registration, and ensure the proper use of PII coding as required by the protocol. Robotic Process Automation (RPA) parses the file name and confirms proper formats and inclusion of the required fields. Additionally, the DPA can test against existing data registered to ocuVault to ensure that the submitted data is consistent with expectations. This automated validation can dramatically reduce errors associated with manual validation that may occur days or weeks after imaging and test data is submitted.

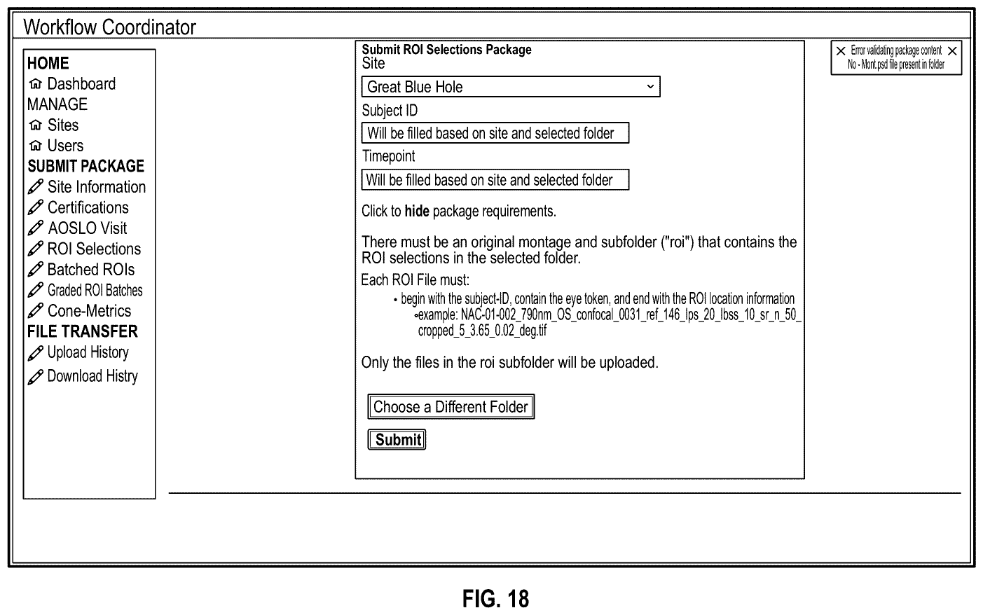

FIG. 18 illustrates an example step in the retina imaging process, involving the extraction and submission of ROIs from a visit montage, in accordance with some embodiments of the inventive concepts. FIG. 18 presents a screenshot of a subsequent step in the workflow of the retina imaging process that is one embodiment of the present inventive concept. This is the extraction of ROIs from an imaging Visit Montage. A User with an appropriate Role downloads a Visit Package from ocuTrack, extracts ROIs, and submits the plurality of ROIs in a folder with the Visit montage. Each ROI has information specific to that ROI, including location information with respect to a landmark (usually the fovea in this instance) of the retina. The ROI naming convention reflects the required ROI-specific data. Validation testing occurs prior to upload allowing for corrective action, as with each other process step.

In some embodiments of the present inventive concepts, ROI selection is one of any number of pre-processing operations that may executed prior to advancing the data to a subsequent step. In some embodiments of the present inventive concept, ROI selection is a human mediated step supported by algorithms in an associated software application. For example, ocuLytics is a software application that facilitates ROI selection in many ways, including identifying boundaries of ROIs within the Visit montage, and outputting ROIs to a folder with a naming convention consistent with upload validation requirements. Such software may be desktop software that requires download of images for local operation, or may be a web application, or other without deviating from the intent of the present inventive concept.

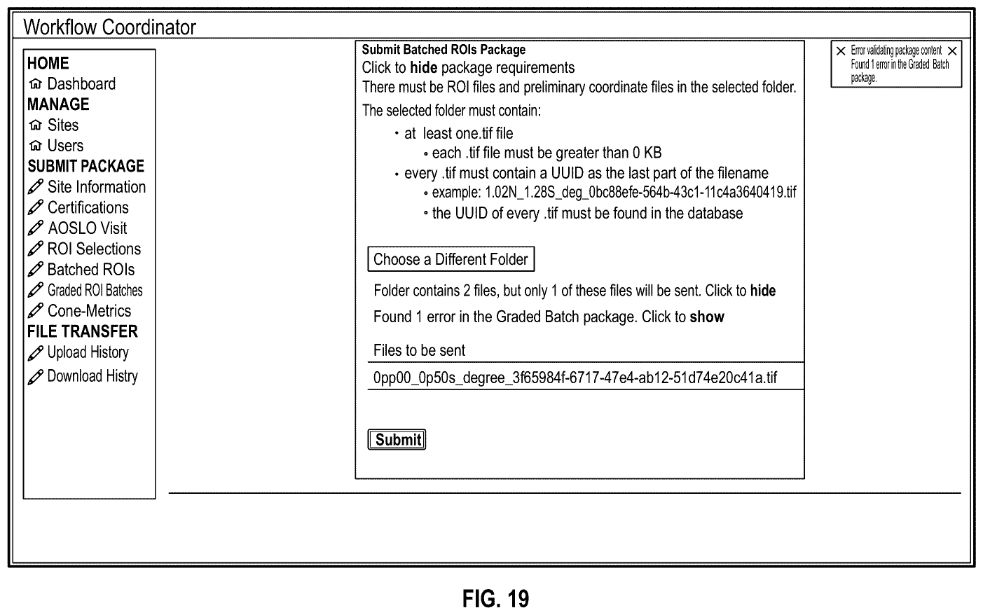

FIG. 19 illustrates an example user interface screenshot for uploading a Package of batched ROIs in accordance with some embodiments of the inventive concepts. FIG. 19 presents a screenshot for upload a Package of Batched ROIs. As discussed previously, an embodiment of the present inventive concepts includes Robotic Batch Process Automations for batch creation. In another embodiment of the present inventive concept, batching of masked and randomized images may be performed manually. In this circumstance, the submittal of Batches will follow validation rules similar to rules that guide the RBPA.

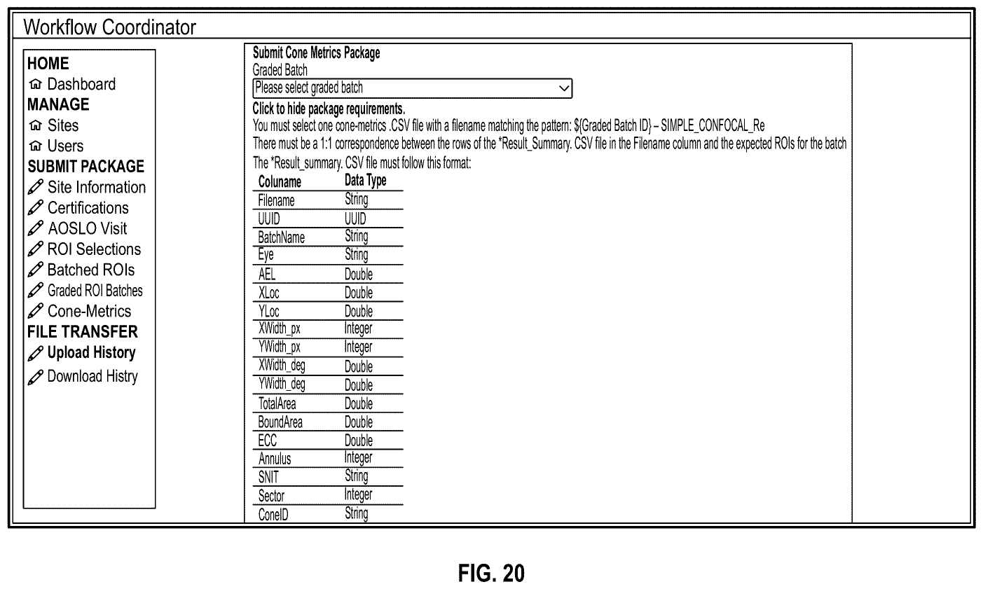

FIG. 20 illustrates an example screenshot for uploading a Package of metrics derived from the grading of ROIs. FIG. 20 presents a screenshot for upload of a Package of Metrics derived from grading of ROIs. In some embodiments of the present inventive concept, a set of coordinate files that define image segmentation or feature identification or the like are uploaded as an output of the Grading process.

For example, after ROI selection and Batch creation, a Grader will mark the presence of cone photoreceptors on each ROI, and the (x,y) locations of each cone are saved to a coordinate file. In another example, a layered anatomical structure such as a retina may be segmented to provide the location of physiological relevant surfaces, and these surfaces may be recorded in a coordinate file. In yet another example, pathological features may be identified and locations, areas, and/or volumes may be recorded. Such coordinate data may be recorded to an annotations database that is linked to, and interoperable with, the source images, and data records, for example in ocuVault. A coordinate file is just the documentary record that a User or a software application may use for subsequent computations. The set of computational outputs that reduce segmented or annotated images to a reduced set of coordinates are Metrics of the image. Spatial metrics such as density, neighbor distances, and variances of these properties are among the metrics used to quantify the distribution of rod and cone photoreceptors in a retina. Similarly, layer thicknesses are among the metrics used to quantify the health of a retina, and fluid volumes are among the metrics used to quantify vascular disease in a retina.

In some embodiments of the present inventive concept, coordinate files are retrieved by a computational User or Grader after grading, metrics are computed with a local software application and metric results are submitted back to ocuTrack, again with appropriate validation criteria. Metric computation may also be readily implemented in a Robotic Batch Process operation.

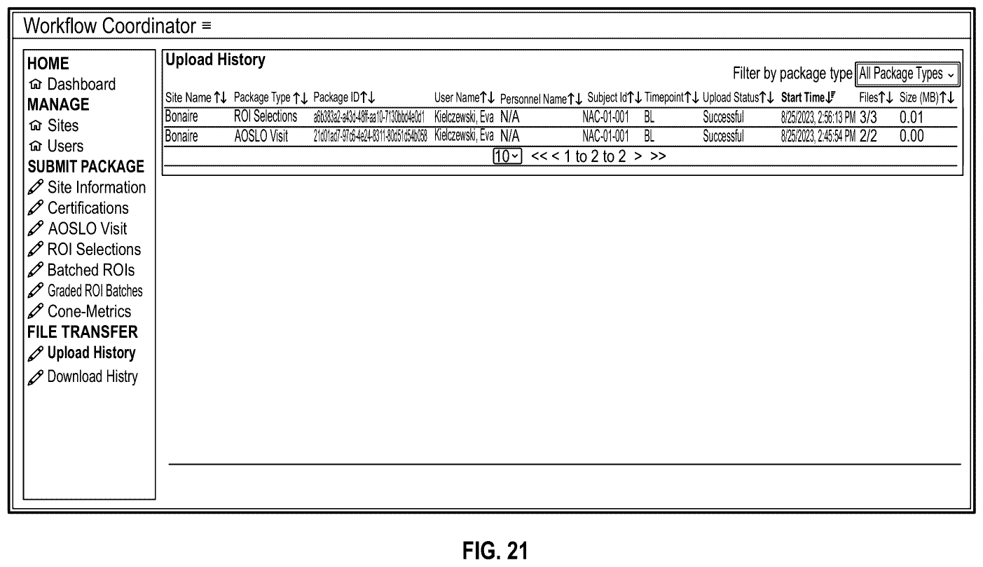

FIG. 21 illustrates example transaction histories available through a sortable view in an example workflow management system, in accordance with some embodiments of the inventive concepts. In some embodiments of the present inventive concept, transaction histories are available through a sortable view with content columns relevant to execution of the workflow, as shown in FIG. 21. In some embodiments of the present inventive concept applied to multi-site studies and clinical trials, the following columns are provided: 1. Site Name; 2. Package Type; 3. Unique Package ID; 4. User (who submitted Package); 5. Personnel Name (e.g. on behalf of); 6. Coded Subject ID (no PII or PHI); 7. Timepoint; 8. Upload (or Download) Status; 9. Start Date and Time; 10. Number of Files Submitted; 11. Size (e.g. MB) of submittal Package.

In a further embodiment of the present inventive concept, additional dashboards with tracking information are available to Users with appropriate Roles and Team membership.

In a further embodiment of the present inventive concept, a directory view is available that allows direct visualization and access to objects and records in customary hierarchical structures. The choice of hierarchy may be selected by the User for specific tasks. For example, the following hierarchical orderings may be readily configured by drawing on the ocuVault database architecture:
1. Longitudinal View for Subject: Subject\Timepoint\Package Type\{data}
2. Cross-Sectional View of Metrics at Timepoint: Package Type\Timepoint\Subject\{data}
3. Quality Control of User Grading: Package Type\Timepoint\User\{data}

A key feature of the present inventive concept is the replacement of file sharing through directories and folders with workflows and automations that can handle the inherent complexities of images, unstructured data, and analysis in research studies and clinical trials. Kanban boards can be used in project management and can be adopted for image processing workflow management, incorporating both human actions and Robotic Process Automation.

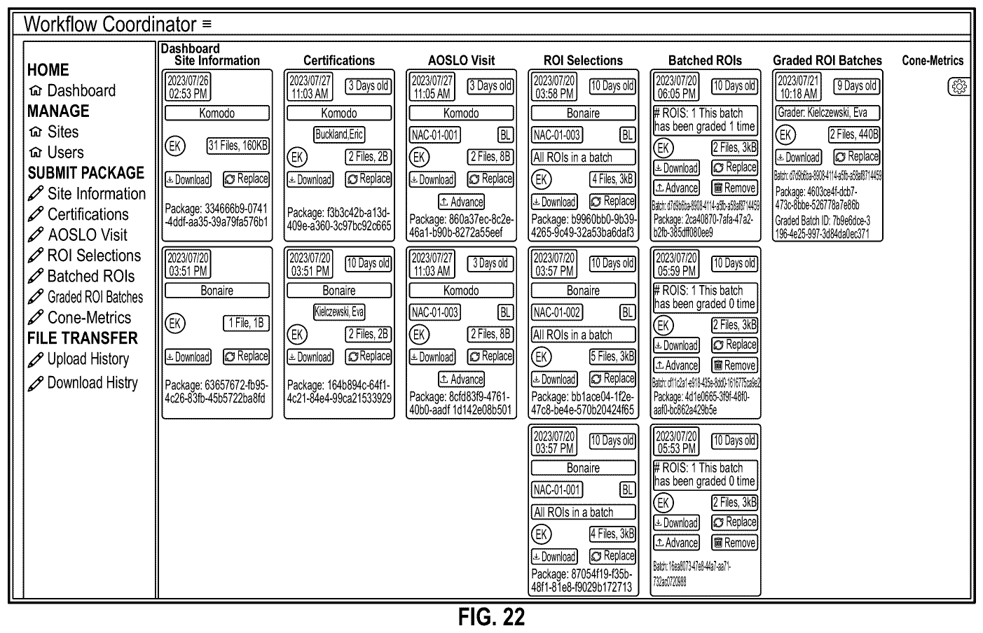

FIG. 22 illustrates an example dashboard with a series of Kanban cards representing different stages of an image processing workflow, in accordance with some embodiments of the inventive concepts. The first column in this dashboard may represent the submission of site information, while subsequent columns track stages of personnel certifications, imaging visits, ROI selection, batch processing, grading, and cone metrics generation. Each card on the dashboard reflects a task or action appropriate to a particular stage in the workflow, indicating the current state of activity for each respective item. Each card is associated with one or more Packages, and this defines actions and routing instructions for a package at a particular stage in the workflow.

FIG. 22 shows an example dashboard of FIG. 14 with a series of Kanban. From left to right, the first column is for Site Information, the second column is for Personnel Certifications, followed by (AOSLO) Imaging Visit, ROI Selection, Batched ROIs, Graded ROI Batches, and Cone Metrics.

Each Kanban Card is tailored to the intent of the workflow at the respective column. The first two columns are Single-Step processes. Content is to be Submitted. The content may be Downloaded or Replaced. There is not an intent to Advance the Content to a subsequent process step. The Kanban Cards do not present an Advance function option.

When a User submits a Site Information Package, as shown in FIG. 16(B), a Site Information Kanban Card is created. This informs personnel that Site Information is available, and the associated Package may be Downloaded or Replaced directly from the Kanban Card. The Site Information Kanban Card includes, from top to bottom, left to right: the date and time of the submittal; the Site of the submittal; the User making the submittal; the number and size of files submitted; an Action button to Download; an Action button to Replace; and a 32 character Universally Unique ID code (UUID) for the Package.

When a User submits a Certification Package, a Certification Kanban Card is created. This informs personnel that a personnel Certification is available, and the information may be Downloaded or Replaced directly from the Kanban Card. The Certification Kanban Card includes, from top to bottom, left to right: the date and time of the submittal; the age (days since submittal) of the Kanban Card; the Site of the submittal; the name or ID of the person certified; the User making the submittal; the number and size of files submitted; an Action button to Download; an Action button to Replace; and a UUID for the Package. Aging information is supported by a process automation to draw attention to information that has not been reviewed within a specified time window. For example, the aging button may turn from green to red, and/or a message may be transmitted by email, voicemail, SMS, or direct messaging to the responsible User(s) who may need to act on the aged information.

When a User submits an (AOSLO) Imaging Visit Package, an AOSLO Visit Kanban Card is created. This informs personnel that an image (in this case a montage) is available for action, and the information may be Downloaded or Replaced directly from the Kanban Card. The Visit Kanban Card includes, from top to bottom, left to right: the date and time of the submittal; the age (days since submittal) of the Kanban Card; the Site of the submittal; the coded ID of the subject; the imaging timepoint (for a longitudinal study or clinical trial); the User making the submittal; the number and size of files submitted; an Action button to Download; an Action button to Replace; and a UUID for the Package. Aging information is supported by a process automation to draw attention to images that has not been reviewed within a specified time window.

The Kanban Card of column 3 (AOSLO) Imaging Visit also includes an Advance option. Advancing a Kanban Card from column 3 invokes the Submit ROI Selections Package shown in FIG. 18, pre-populating the web form based on the initiating Kanban Card. When the ROI Selections Package is uploaded, process automation creates the Kanban Card in column 4 for next step in the process, informing appropriate User(s) of the availability of ROI Selections.

It is noted that there is not an Advance Action associated with the ROI Selections Kanban Card. The reason for this is that the ROI Selections may be masked and randomized into Batches for Grading. When the ROIs are folded into a Batch Package, a notation on the Kanban Card includes a message of on the allocation of ROIs to Batches. When all ROIs have been allocated to grading Batch Packages, the Kanban card will read “All ROIs in Batch.” RBPA performs the batching and provides immediate visibility to authorized Users that ROIs are appropriately included in at least one grading batch.

Batches that are ready for Grading appear as Kanban Cards in column 5. These Batches are presented to Users with the Grading role for action. It is common to instruct multiple Graders to analyze images for reproducibility purposes. The Batched ROI Kanban Card indicates the number of ROIs in the batch, and the number of Graders that have completed Grading the batch.

Upon completing the grading of a batch, a Grader will Advance the Kanban Card to column 6, Graded ROI Batches, invoking a web form for submittal of the graded batch. Each batch has its own UUID which will differ from the Package containing the Batch. Once a batch is Graded, the graded batch will also have its own UUID, providing traceability to specific Graders. The Graders name is present on the column 6 Graded Batch Kanban Card.

Finally, Metrics are computed from the graded ROIs using RBPA, and completed metric reports will show in column 7.

Additional features of ocuTrack and the ocuTrack web interface include process steps and dashboards for descriptive statistics, pooled statistics for cross-sectional and longitudinal analysis, test-retest statistics drawn from repeated grading of ROIs as discussed in the batch creation process, and control charts for inter-grader reproducibility, and consistency of results of Gold Standard images as processed by multiple graders multiple times.

A specific embodiment of the present inventive concept involves the grading of high-resolution retinal images for photoreceptor topography analysis. It is a further object of the present inventive concept to generalize the workflow for other multi-step data analysis processes. It is still a further object of the present inventive concept to create custom workflows with a graphical and lo- or no-code process that draws upon the ontology of the workflow process, supported by the underlying database schema, to meet the requirements of use-case specific study protocols.

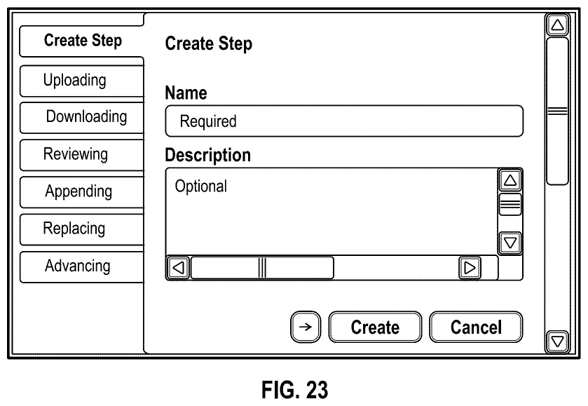

FIG. 23 illustrates the core ontology of workflow process through an organizational user interface for constructing custom workflow elements in a system, in accordance with some embodiments of the inventive concepts. This interface can include features for creating and managing workflow steps, setting validation requirements, and linking steps to various actions within the broader workflow process. The system allows users to build workflow steps through a graphical or low-code/no-code interface, ensuring flexibility for different research or trial protocols. The construct includes tabs with structures for building the workflows, process steps, and Kanban Cards for the following actions: i) Create Step; ii) Uploading content; iii) Downloading content; iv) Reviewing content; v) Appending content; vi) Replacing content; and vii) Advancing content to a subsequent step. The first step is to Create Step.

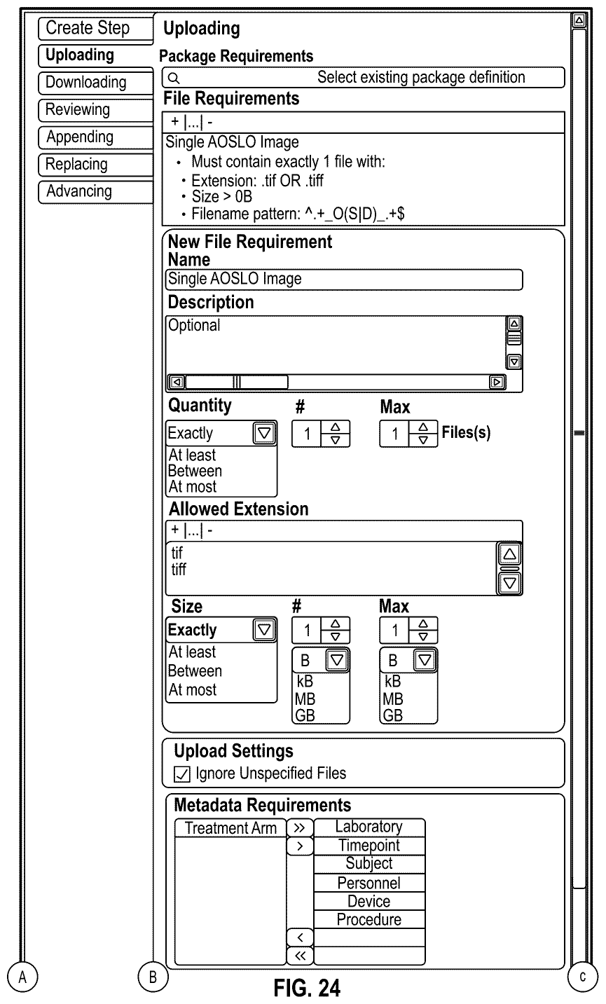

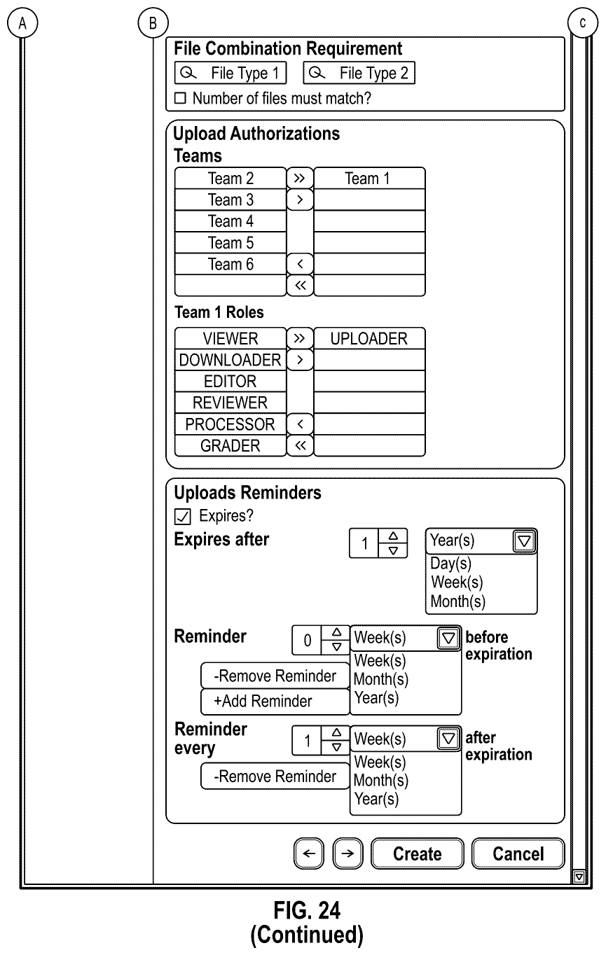

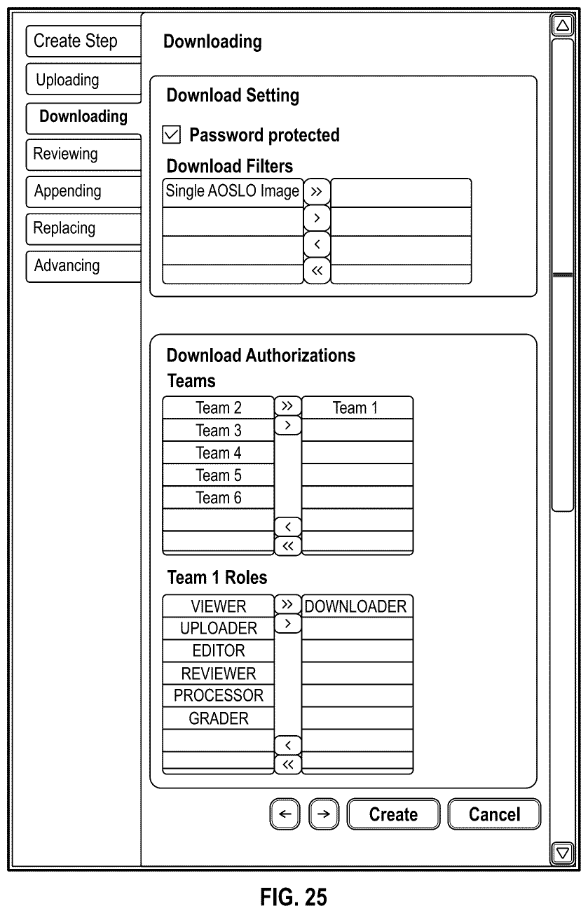

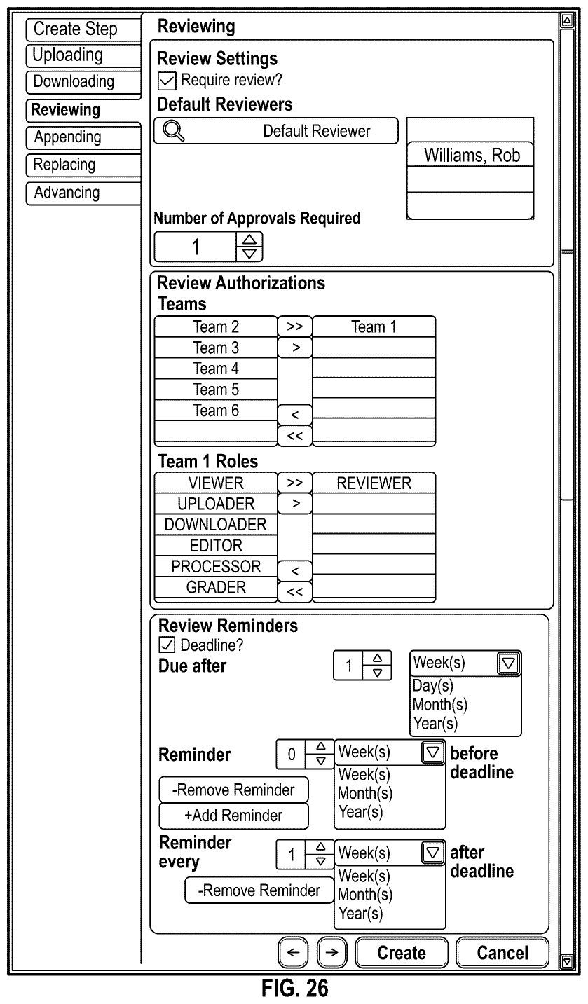

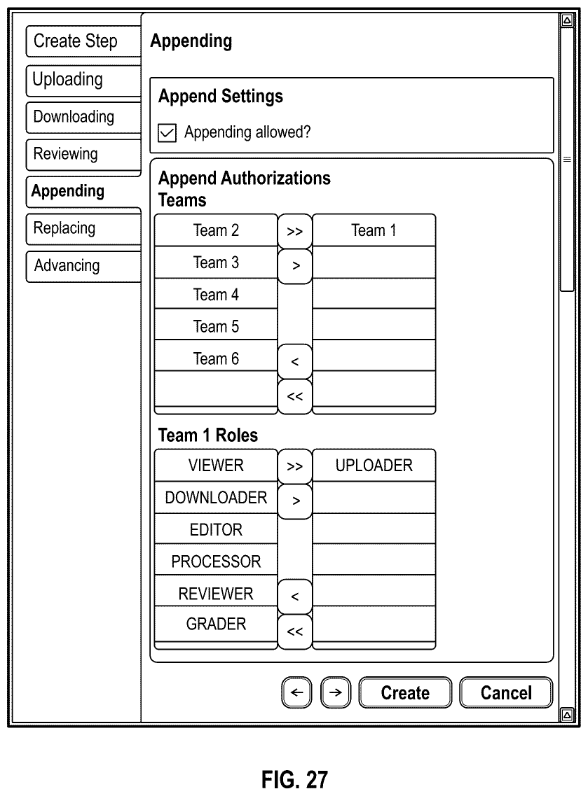

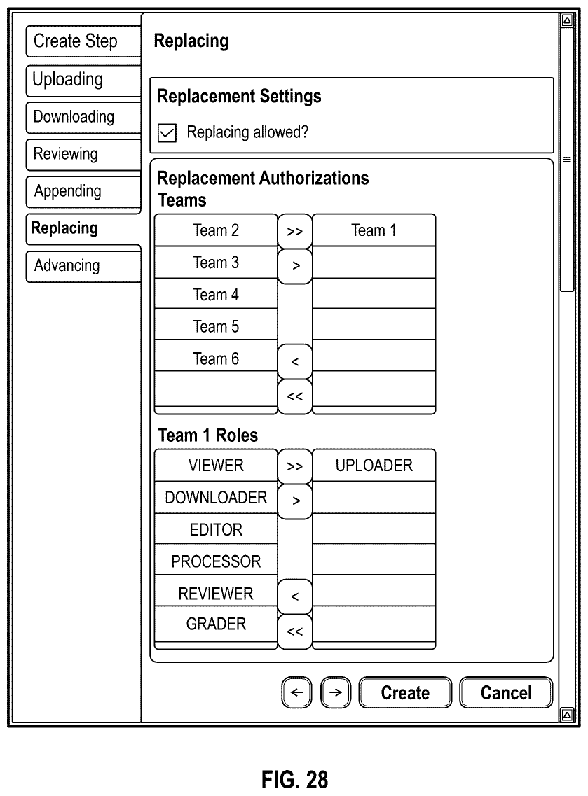

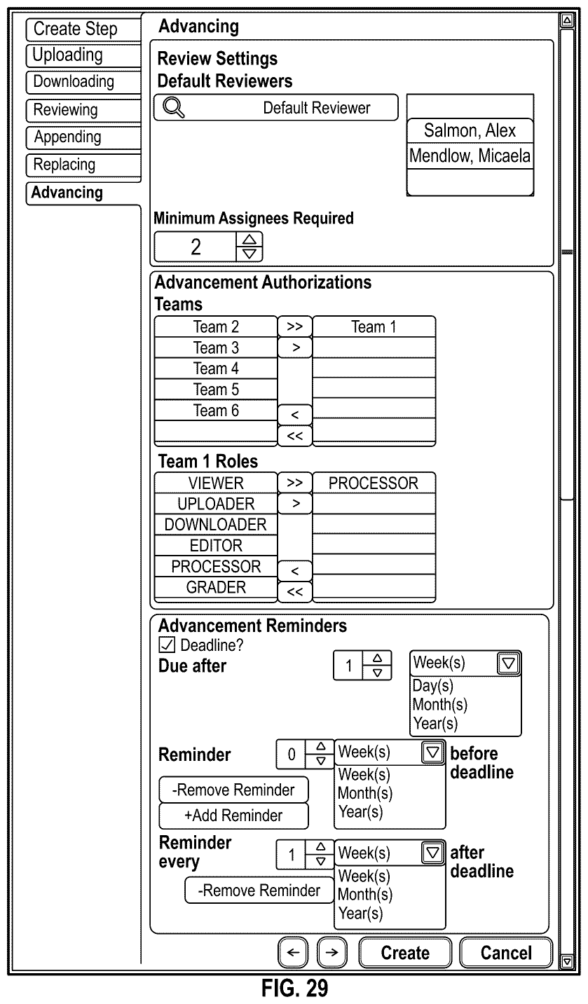

FIGS. 24-29 show UI interfaces for designing the requirements and behaviors for each step in the workflow, further defining the workflow ontology. The Package Upload requirements stack is shown in FIG. 24, and includes components for setting File Requirements, Metadata Requirements, Authorizations, and Reminders. Package Download requirements are shown in FIG. 25, and include password protections, filters, and authorizations. Package Review requirements, shown in FIG. 26, include flag for requiring a review, setting default reviewers and number of approvals required, setting authorizations, and scheduling reminders. Package Appending and Replacing requirements, shown in FIG. 27 and FIG. 28, respectively, include flags for allowing content appending/replacing, and setting authorizations. Workflow Advancing requirements, shown in FIG. 29, include specifying the default assignees, number of assignees, authorization, and scheduling reminders.

FIG. 24 illustrates example Package Upload requirements stack and includes components for setting file requirements, metadata requirements, authorizations, and reminders. In some embodiments, the user interface allows users to define specific file types, metadata fields, and permissions required for each stage of the upload process. Automatic validation rules can be enforced before allowing a Package submission to proceed.

FIG. 25 illustrates example Package Download requirements, including password protections, filters, and authorizations. This interface ensures that data access permissions are tightly controlled, enabling specific users or groups to access certain Packages based on their role or project includement.

FIG. 26 illustrates example Package Review requirements, including flags for requiring a review, setting default reviewers, and defining the number of approvals required. In this system, Packages can be routed to designated reviewers for validation before further progression in the workflow. The system may also provide reminders for reviews pending for a set period.

FIGS. 27 and 28 show example Package Appending and Replacing requirements, respectively, including flags for allowing content appending or replacing, and setting authorizations. These functionalities ensure that the Package content can be adjusted or replaced according to workflow needs, with the necessary permissions in place to prevent unauthorized modifications.

FIG. 29 shows example Workflow Advancing requirements, including specifying the default assignees, number of assignees, authorization, and scheduling reminders. In some embodiments, the workflow system allows a defined process step to be automatically advanced based on predetermined criteria, such as completion of a previous task or submission of a particular dataset.

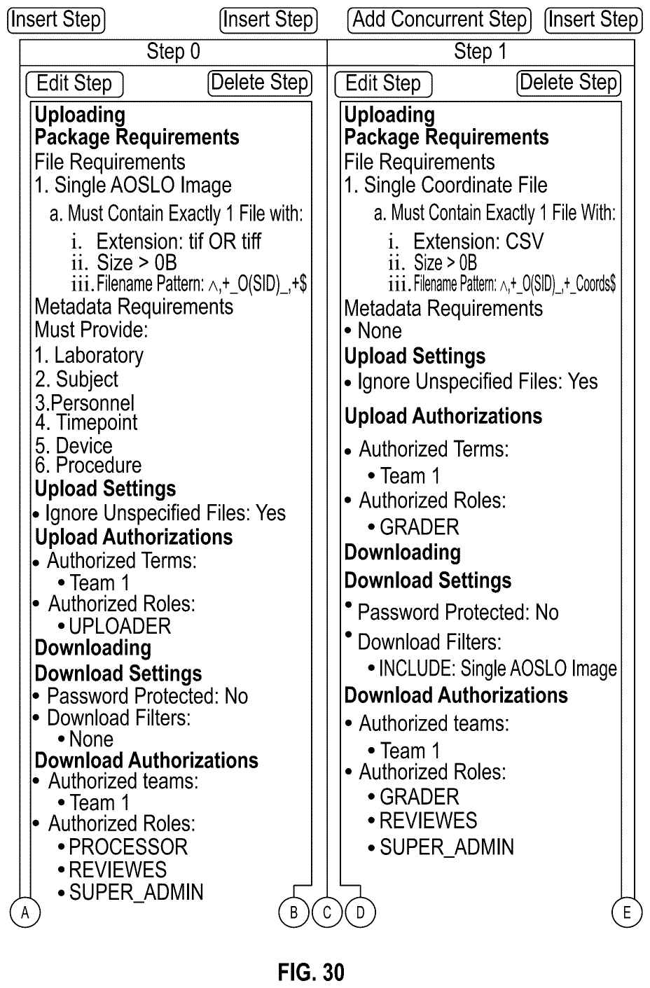

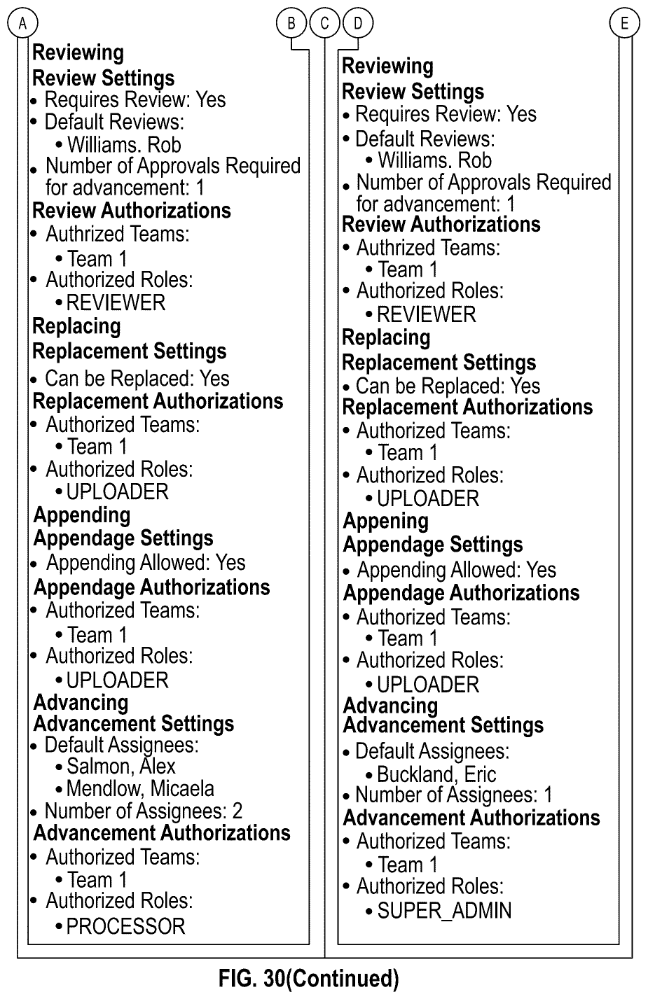

FIG. 30 shows an example workflow ordering model consistent with the workflow ontology in accordance with some embodiments of the inventive concepts. The model may allow for concurrent or sequential steps within a workflow, ensuring that complex, multi-stage processes can be easily visualized and managed. Users can insert, reorder, or configure steps to match the needs of specific research or clinical protocols.

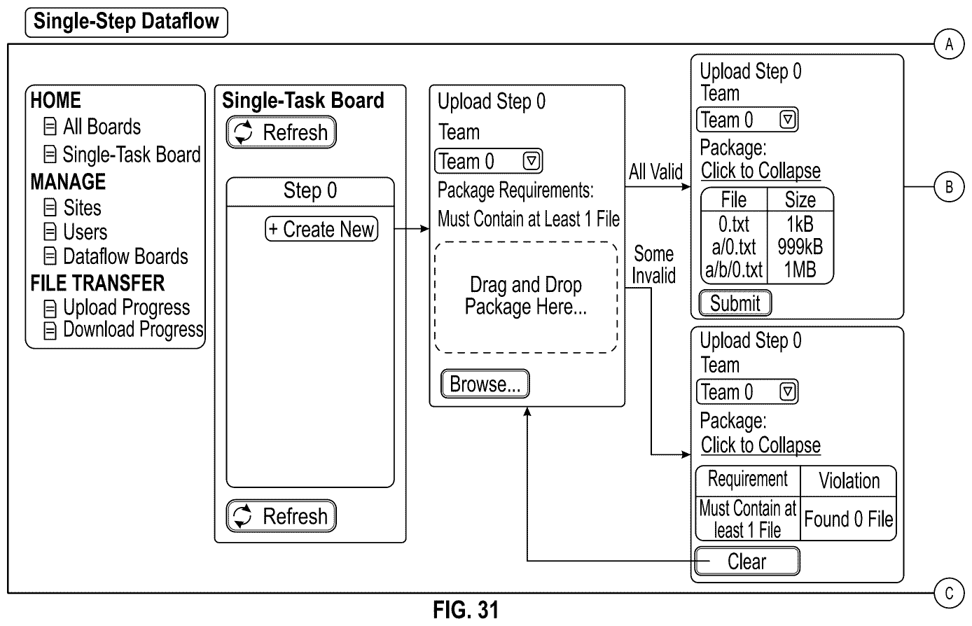

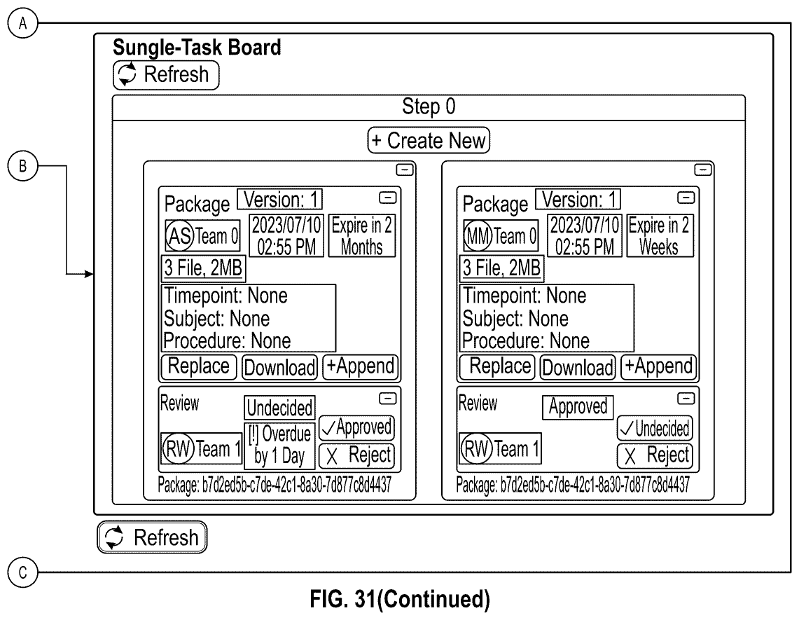

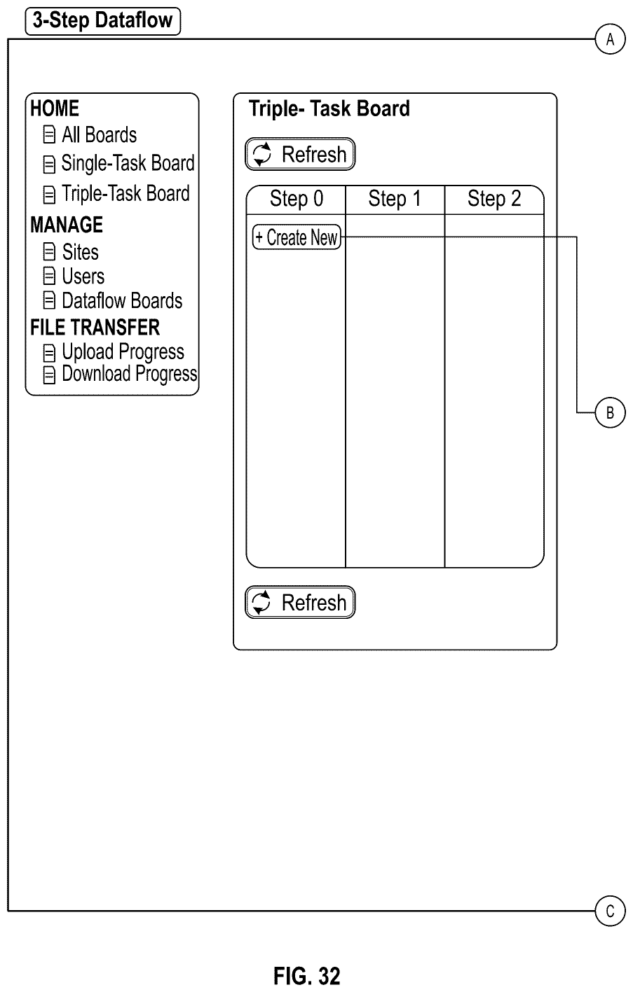

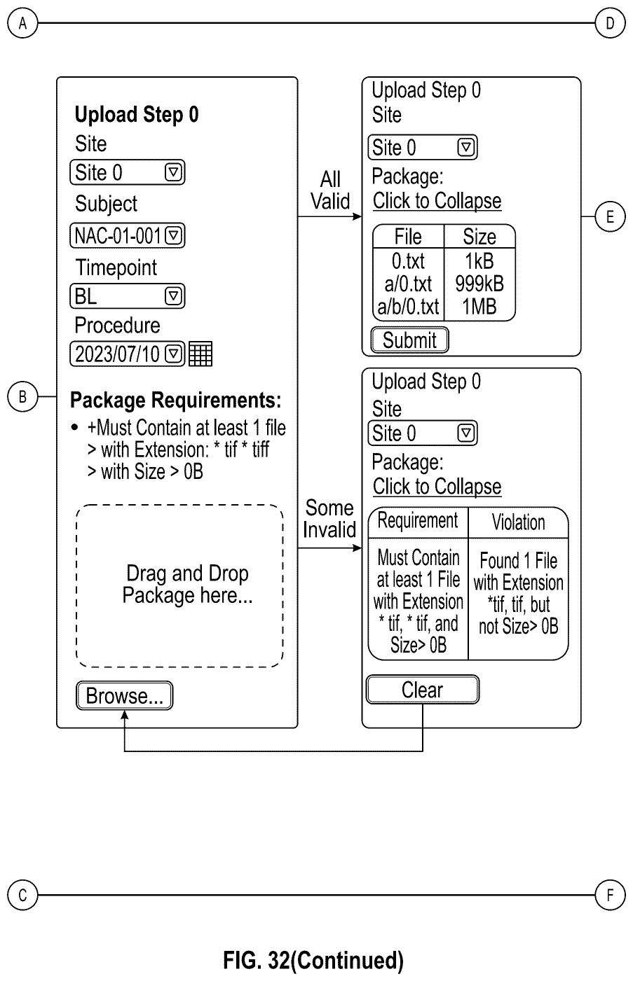

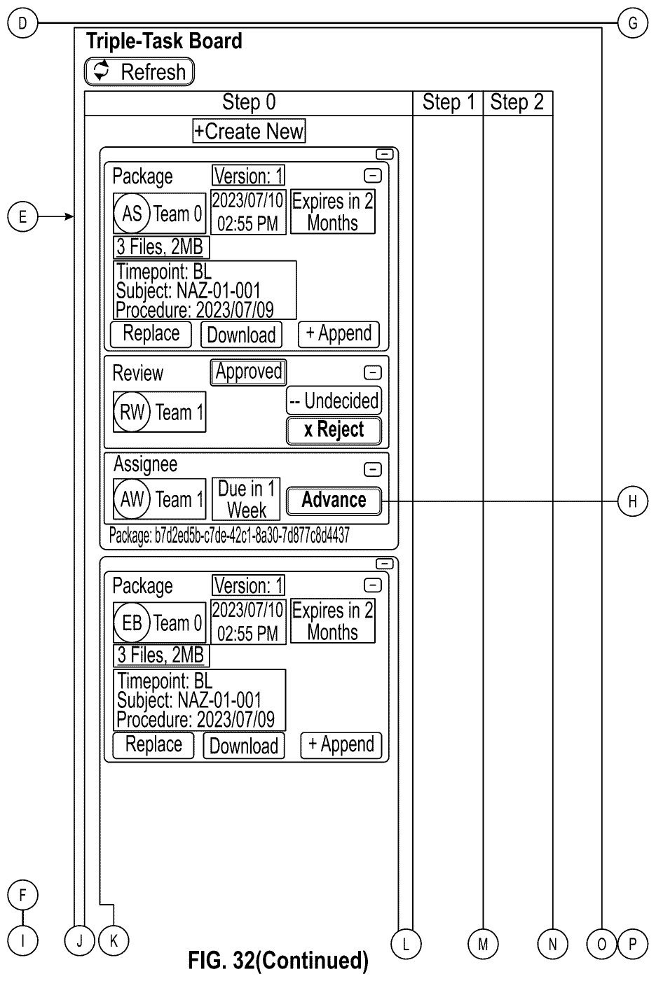

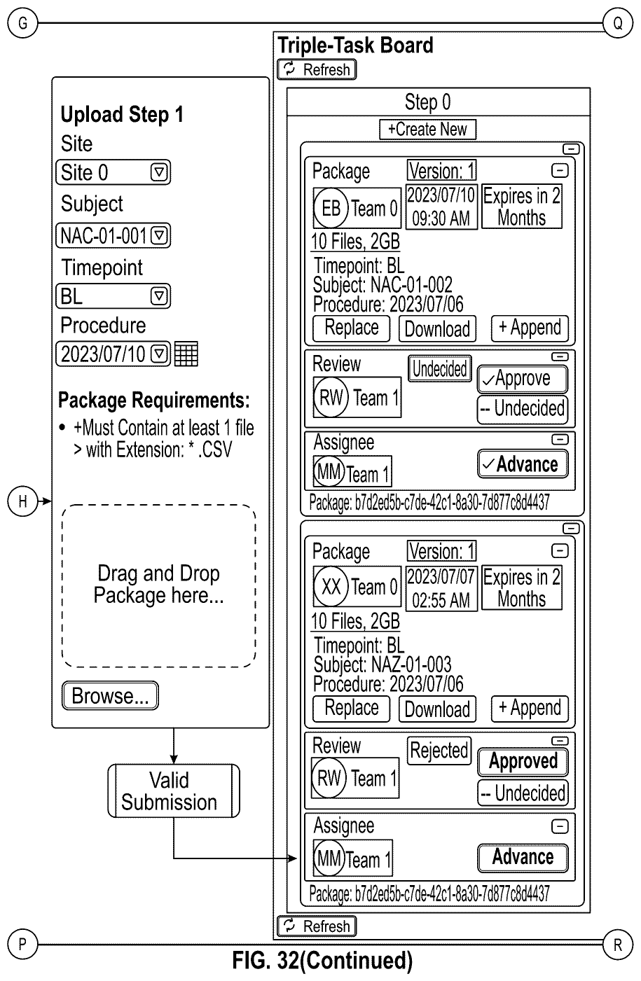

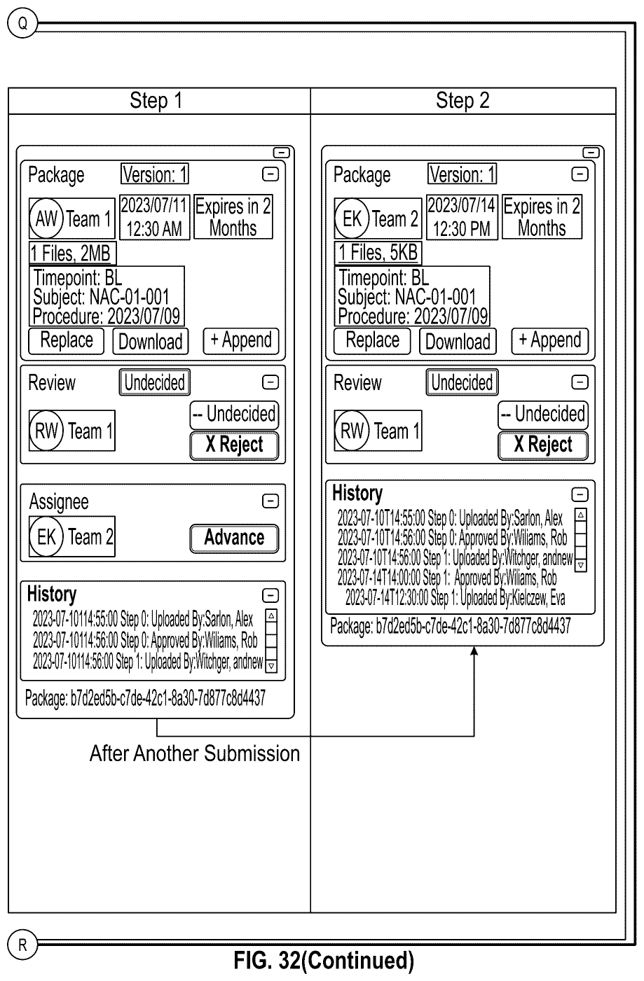

FIGS. 31 and 32 illustrate example UI design elements for a no-code process for establishing a custom workflow and setting validation rules in both single-step and multi-step workflows, respectively. These validation rules ensure that each workflow step adheres to the defined process requirements, including file types, metadata completeness, and role-based access controls. This system can minimize errors and ensure consistency across all data submissions and workflow tasks.

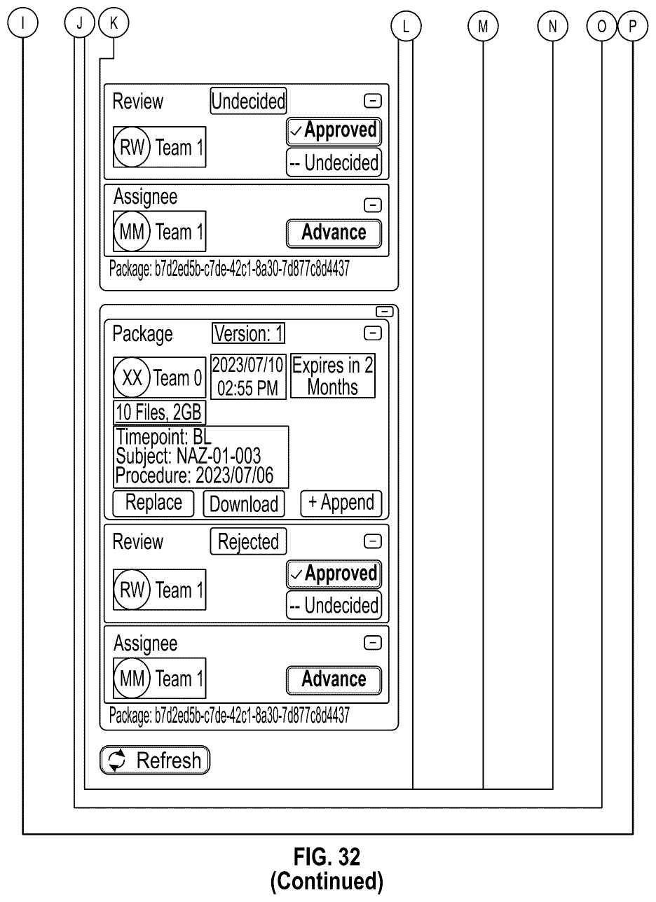

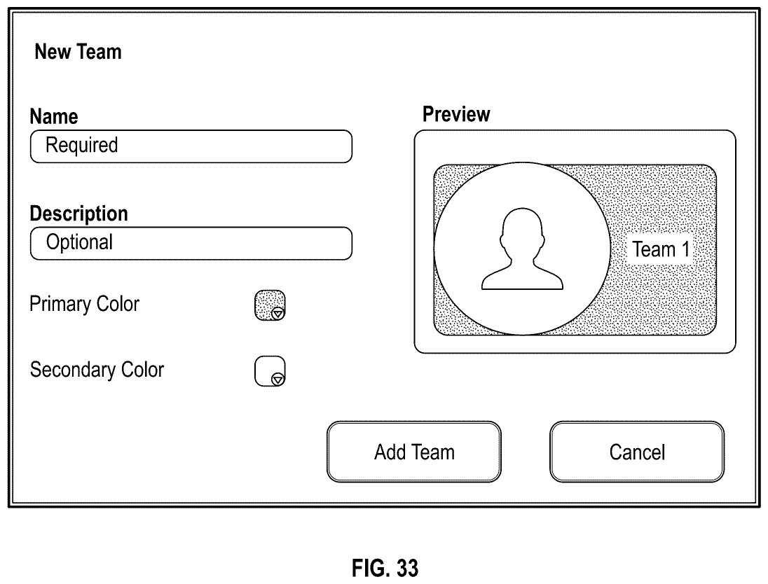

FIG. 33 illustrates an example UI element for establishing new Teams. Teams can be created with assigned roles, and permissions may dictate the specific actions each team or team member can perform within the workflow. This feature ensures that data access and process execution are restricted based on user roles and study needs.

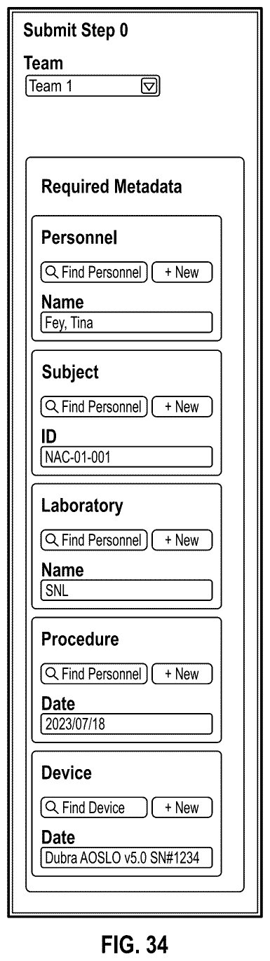

FIG. 34 illustrates an example UI element for filling in missing metadata at a submittal step. This feature can assist users in completing required fields before advancing a workflow step, ensuring that all necessary information is present for downstream processes.

The workflows and rules are built in a no-code environment by setting attributes and requirements in re-usable modules as have been outlined in FIGS. 23-34. Each module is stored in a workflow database. Each module with specific settings is given an UUID for traceable linking of workflows and Packages. Modules may be combined and re-combined to make systems of workflows that meet specific user objectives, again while maintaining complete traceability of the application of workflow modules to data processing activities.

The workflow modules may allow the inclusion of programming scripts or code to build additional functionality in lo-code context. Such scripts may include file renaming, image pre-processing, messaging, triggers to external actions, and the like.

The system described provides a robust framework for managing complex workflows, especially in environments involving medical research, clinical trials, or data-intensive projects. The combination of cloud-based data management, configurable workflows, and role-based access ensures the system's flexibility, scalability, and data security. The inventive concepts presented enable efficient, automated, and transparent workflows while allowing for human oversight and intervention where needed. The system can be adapted to various domains of data analysis, research, and clinical processes without deviating from the inventive concepts described.

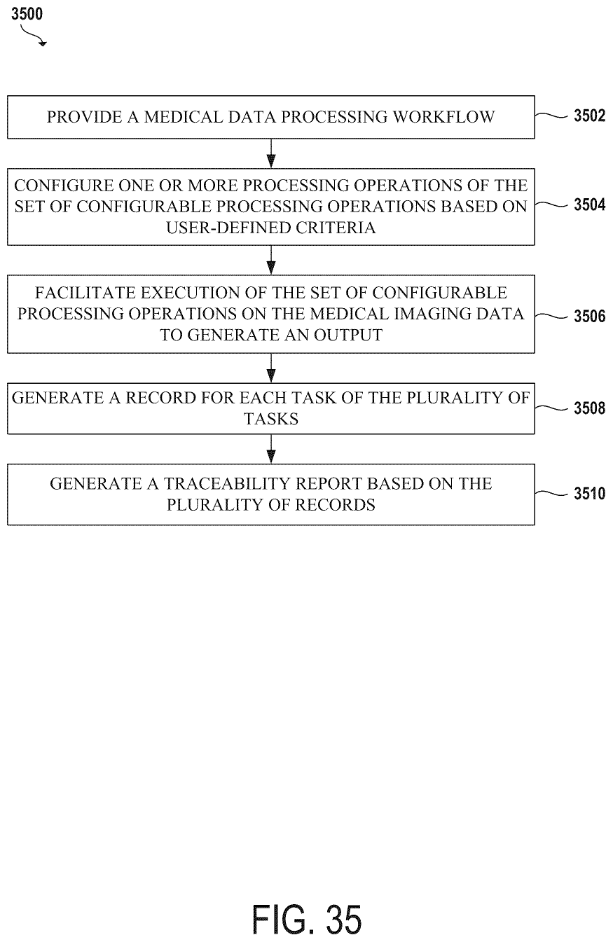

FIG. 35 is a flow diagram illustrative of an embodiment of a routine 3500 for managing a configurable medical data processing workflow. Although described as being implemented by the workflow coordinator 220, it will be understood that the elements outlined for routine 3500 can be implemented by any one or a combination of computing devices/components that are associated with the workflow environment 200 such as, but not limited to, the data intake system 210 data intake system 210, the data store system 230, the visualization interface system 260, the data analysis system 270, or the reporting system 280. Thus, the following illustrative embodiment should not be construed as limiting.

At block 3502, the workflow coordinator 220 can provide a medical data processing workflow, which can include a set of configurable processing operations. These operations can, in some cases, be pre-defined, user-defined, or a combination of both, providing flexibility in adapting the workflow to process different types of medical imaging data, such as CT scans, MRIs, retinal imaging, or ultrasound data. The set of configurable operations can include tasks such as image segmentation, enhancement, feature extraction, and data transformation. By offering this workflow, the workflow coordinator 220 can allow users to define and manage the flow of data through various stages, enabling the medical imaging data to be processed in a way that aligns with specific clinical or research objectives. In some cases, this adaptability can help accommodate different imaging types and processing needs, ensuring that the workflow remains customizable for various medical applications.

In some cases, the workflow can be implemented as a structured system for managing medical data processing operations, where graphical user interface (GUI) elements can be presented to facilitate various stages of the workflow. These GUI elements may correspond to actions such as selecting specific processing operations from a predefined set or adjusting parameters associated with medical imaging data. For example, the GUI may display dropdown menus or sliders for selecting or modifying tasks without requiring complex programming. In some cases, this interaction can be implemented as a low-code or no-code solution, allowing users to make necessary adjustments efficiently. This approach can provide advantages by simplifying the customization of workflows for users, such as medical staff or researchers, without the need for advanced technical skills.

The workflow can be designed to present tasks in a logical sequence, distinguishing between fully automated tasks and operator-assisted tasks, such as reviewing or annotating imaging data. For instance, when user input is needed to annotate an image, the system can display annotation tools, such as markers or text fields, to assist in documenting observations. As tasks are completed, the system can be configured to automatically update the status of the workflow, providing ongoing feedback.

At block 3504, the workflow coordinator 220 may configure one or more processing operations based on user-defined criteria. The configurable nature of the workflow can provide significant advantages, including adaptability and ease of use. By allowing users to define and manage operations tailored to specific clinical or research objectives, the system may support a wide range of medical imaging applications, such as diagnostics, treatment planning, and clinical trials. This flexibility can make the workflow suitable for a variety of imaging modalities and clinical protocols, accommodating both standard and unique medical scenarios. In some cases, this approach can reduce the need for extensive custom programming, making it accessible to users with varying technical expertise.

In some cases, the user-defined criteria may include the selection of at least one processing operation from a plurality of predefined processing operations. These operations may be selected from a library of tasks, such as image segmentation, noise reduction, or feature detection. For example, a user working with retinal images may select a segmentation algorithm designed to identify specific retinal layers. This predefined selection process may streamline setup, allowing users to quickly configure the workflow with established, proven techniques for processing the medical data.

In some cases, the user-defined criteria may include an indication of adjustments to one or more parameters associated with the selected processing operations. The configuration of these parameters may be facilitated through a graphical user interface (GUI) that provides sliders, dropdown menus, or similar input mechanisms. For example, a user may adjust the sensitivity of an image filter or alter the number of iterations used by a machine learning model. Such adjustments can offer finer control over the processing tasks, allowing the workflow to be precisely tailored to the needs of a particular dataset. Such functionality can be useful when adjusting processing operations for high-resolution MRI or CT scan data, where minor parameter changes can significantly impact the final output.

In some cases, the user-defined criteria may allow for the definition of entirely new processing operations. This feature may enable users to specify custom algorithms or procedures that are not available in the predefined library. For instance, a research team studying a rare medical condition may define a novel algorithm to analyze unique biomarkers or anatomical features present in their imaging data. By supporting the creation of new operations, the system can provide a highly flexible platform capable of evolving with ongoing advancements in medical research and technology.

At block 3506, the workflow coordinator 220 can facilitate the execution of the configured processing operations on the medical imaging data to generate an output. The execution can involve a combination of computer-automated tasks and operator-assisted tasks. Computer-automated tasks can include operations such as image filtering, segmentation, or the application of machine learning algorithms for feature detection, while operator-assisted tasks can involve manual actions, such as reviewing images for anomalies, annotating regions of interest, or confirming computational results. Each task, whether automated or assisted, can result in either a transformation of the medical imaging data (such as altering its structure or format) or the addition of annotations (such as labeling specific regions for later analysis). The ability to combine both automated and manual tasks allows for an optimized workflow that balances efficiency and human expertise, ensuring that complex cases can be handled with expert oversight while routine tasks are automated for speed and accuracy.

At block 3506, the workflow coordinator 220 can facilitate the execution of the configured processing operations on the medical imaging data to generate an output. The execution may involve a combination of computer-automated tasks and operator-assisted tasks, enabling a flexible workflow that integrates both machine-driven processes and human expertise. Computer-automated tasks can include operations such as image filtering, segmentation, or the application of machine learning models for feature detection. These tasks can handle repetitive or computationally intensive actions efficiently, potentially reducing manual effort.

In some cases, operator-assisted tasks can be included. Operator-assisted tasks may include manual actions, such as reviewing images for anomalies, annotating regions of interest, or verifying computational results, allowing human expertise to be integrated into the workflow where precision and judgment are required. These tasks can involve expertise that complements automated algorithms, such as identifying subtle anomalies or making decisions based on clinical judgment.

Combining both automated and manual tasks within a unified workflow can provide the benefit of maintaining traceability across all stages. In some cases, this can address the challenges of traditional workflows, where operator-assisted tasks may be handled separately, leading to a lack of continuity in tracking changes. For example, a continuous record of both automated and manual tasks can be maintained. This allows all actions applied to the medical imaging data to be documented, providing an audit trail that supports transparency and accountability. This traceability can be beneficial in regulated environments where detailed records are important for clinical or regulatory compliance.

At block 3508, the workflow coordinator 220 can generate a record for each task within the plurality of tasks performed during the workflow execution. Each task, whether automated or assisted, can result in either a transformation of the medical imaging data (e.g., changing its structure or format) or the addition of annotations (e.g., labeling specific regions for further analysis). Each record can include a unique identifier (such as a universally unique identifier or UUID) corresponding to the specific task and can contain detailed information about the task, including its nature (e.g., transformation or annotation), the time it was performed, and whether it was completed by an operator or automatically by the system.

In some cases, when tasks result in the generation of sub-tasks, the workflow coordinator 220 can generate hierarchical records that reflect the parent-child relationships between the task and its associated sub-tasks. These records can be stored in a structured manner, allowing them to be retrieved for later analysis, auditing, or compliance purposes. The ability to track every task in detail ensures that each step in the workflow is fully documented, contributing to transparency and accountability throughout the medical data processing pipeline.

At block 3510, the workflow coordinator 220 can generate a traceability report based on the plurality of records. The traceability report can provide a complete, documented history of all operations performed on the medical imaging data, including a chronological sequence of the tasks, the unique identifiers associated with each task, details of any transformations or annotations applied, and the identity of the operator or system responsible for each task. The traceability report can serve as a critical tool for compliance with regulatory requirements, such as those related to clinical trials, patient data management, or quality assurance protocols in medical imaging workflows. In some cases, the traceability report can also include audit logs of user interactions, where each user interaction (such as modifying a workflow parameter or annotating an image) is logged with a unique identifier and the credentials of the user, providing full accountability.

In some cases, the traceability report can be organized into a hierarchical structure that reflects the relationships between tasks and sub-tasks, assigning unique identifiers to each. For example, a hierarchical structure can facilitate more efficient tracing of workflow actions by grouping related tasks under a common parent task. For example, if an imaging dataset is split into smaller segments for separate analysis, the traceability report can link each of these segments back to the original dataset, providing a clear map of how the data was processed. The hierarchical organization can also make it possible to recreate the exact sequence of operations in the workflow, ensuring that the workflow can be reproduced to generate an identical result. This capability can be important for facilitating reproducibility in medical research or for validating the consistency of clinical diagnostic processes. By following the sequence of UUIDs and task records, a user or system can trace the full processing path of the medical imaging data, from its initial state through every modification and annotation.

The workflows and traceability reports may also include iterative processes. In such iterative processes, the workflows allow the return of a modified Package from a downstream step in the workflow to an upstream step in the workflow. An example of such an iterative process may be in the form of a human quality control step, where a modification or correction is made and the object in question is returned for reprocessing after such intervention. Another example may include an iterative process for segmenting an image, such that an automated segmentation is applied to an image in a workflow step A, corrected in a workflow step B, directed to a batch of similarly corrected segmentations for segmentation re-training step C, and where the image is reprocessed in workflow step A using an updated segmentation algorithm.

### Methods and Systems for Determining Clinical Trial Eligibility Based on Retinal Image Analysis and Biomarkers

An important application of the configurable medical processing system described herein is in the discovery, development, and deployment of robust, quantitative, and objective markers of disease, disease progression, and therapeutic effectiveness. Images of the eye provide a particularly unique opportunity for the development of quantitative imaging biomarkers for eye disease and diseases that are observable through the eye. Oculomics is a recent term adopted to describe the field researching systemic disease through ocular imaging. The eye is a transparent, immune privileged environment that is directly connected to the central nervous system and cardiovascular, as well as the immune, endocrine, and lymphatic systems.

Biomarker discovery is complex, and current processes are inherently complicated, with involvement of images and data, image processing and machine vision algorithms, numerical computations, statistics, and classification algorithms involving a wide variety of participants from biologists and clinical scientists to AI scientists and statisticians, as well as program managers, quality control personnel, and regulatory affairs professionals. Current biomarker discovery processes involve data transformations and data hand-offs between these disparate stakeholders that are difficult to manage, opaque, and lack traceability.

Systematic workflows provide a clear set of processing steps tailored to the problem and the participants make the process less complicated for all stakeholders and makes progress transparent and traceable. Each specific use case for imaging biomarker discovery is unique, requiring workflow configurability described herein. A general pattern for biomarker discovery may be defined by the following steps: Image ingestion and curation; metadata ingestion and association; image pre-processing, annotation, and segmentation; computation of quantitative metrics from segmented images; assessment of correlation among metrics and metadata; classification of subjects according to metrics based on the correlation to relevant metadata.

Development of biomarkers include establishing precision of the marker in a normative situation, the variances associated with subject populations, and the reproducibility associated image device variances and the variance of human interventions in the image acquisition and data processing processes. In an embodiment of the present invention, the configurable workflow processes include an automated or semi-automated statistical engine that receives a batch of metrics from annotated and/or segmented images combined with metadata and computes a set of tests for correlation between metrics and metadata, comparison between manual graders who have annotated or segmented the images, or comparison between manual graders and automated algorithms. The statistical engine may also produce summary statistics for the subject population by metric and by region in the eye. The pools for summary statistics may be narrowed to categories of subjects, for example sex or age, categories of disease or disease stage, or other determinants of health or disease as available in the metadata.

The statistical engine may also include methods for establishing correlation between the various metrics, may perform a principal components analysis to reduce the dimensionality of the metric set to a subset of metrics that are a) weakly correlated among themselves and b) in combination maximally determinant of the disease state for the biomarker is targeted. Further the statistical engine may pool regions of the eye into physiological groups that are likely to have differential response to disease or treatment. In an embodiment of the present invention, the workflow process and statistical engine are configured to identify biomarkers combining at least two weakly correlated metrics evaluated at least two distinct regions of the eye. Such biomarkers offer greater specificity to disease classification and are less susceptible to overfitting.

Managing clinical trials for treating degenerative retinal diseases can involve processing large volumes of medical imaging data, including retinal images that reflect the structure of cone photoreceptors. Traditionally, patient selection for these trials has been based on broad clinical parameters that may not fully utilize the available data for precise stratification. This reliance on generalized parameters can lead to inefficiencies, increased trial complexity, and difficulties in demonstrating therapeutic efficacy. A more data-driven approach can allow for improved patient selection and trial outcomes by focusing on relevant biomarkers.

Disclosed herein are techniques for determining patient eligibility in clinical trials through the use of quantitative biomarkers derived from retinal image data. Cone photoreceptors are implicated in a large class of degenerative eye diseases and inherited retinal dystrophies. Neuroprotective and gene therapies that are operative on cones require the presence of cones in patients to be effective. Imaging biomarkers based on the spatial statistics of cone photoreceptor topography are therefore first-order predictors of the therapeutic potential of a patient. Retinal image data can be analyzed to compute various quantitative measures of photoreceptor spatial statistics, such as, but not limited to, cone density, cone spacing, and regularity of cone packing. These metrics can serve as objective biomarkers for assessing retinal health and the progression of retinal degeneration. By comparing these metrics with one or more predefined thresholds, patients can be stratified into inclusion, exclusion, or other categories, offering a more tailored approach to clinical trial enrollment and potentially improving both trial efficiency and therapeutic outcomes.

Some inventive concepts described herein relate to the development and use of imaging biomarkers for patient selection in clinical trials. These imaging biomarkers, based on the structural characteristics of cone photoreceptors, can be used in combination with other clinical data to provide a more refined method for identifying individuals with therapeutic potential. The use of these biomarkers can improve patient stratification, support compliance with regulatory standards, and enhance the clinical relevance of trial outcomes.

In certain embodiments, the disclosed techniques are implemented via configurable workflows for analyzing retinal image data. These workflows, as described herein, can enable users to select or adjust analysis methods, modify parameters, or define specific criteria for computing cone photoreceptor metrics. This flexibility allows the analysis process to be tailored to the requirements of a particular clinical trial, supporting the optimization of data processing and patient selection based on individualized retinal health data.

Some inventive concepts described herein relate to generating traceability reports documenting the analysis steps performed on the retinal image data. The traceability reports can include details on the methods used, the parameters applied, and any transformations or annotations made to the data. Such documentation supports the auditing and verification of the data processing workflow and can ensure compliance with clinical trial protocols and regulatory requirements.

Some inventive concepts described herein relate to combining automated and user-driven analysis steps to enable efficient processing of retinal image data while maintaining flexibility for expert review and oversight. Automated tasks, such as calculating cone photoreceptor metrics, can be integrated with manual review steps to ensure that clinical decisions are informed by both data-driven insights and expert clinical judgment.

The use of cone photoreceptor metrics derived from retinal image data can offer several benefits in the context of clinical trials. From a regulatory standpoint, these objective structural metrics can provide a direct link to clinical outcomes, facilitating the stratification of intended patient populations and supporting enhanced regulatory review procedures for degenerative eye diseases. In clinical trial operations, the use of these metrics can improve the inclusion and exclusion criteria, potentially reducing trial costs and decreasing the likelihood of trial failure by selecting patients with a higher probability of therapeutic success. Patient outcomes can be improved through more tailored therapeutic approaches and the potential for personalized dosing based on quantitative analysis.

Various photoreceptor imaging techniques, such as adaptive optics-enhanced scanning laser ophthalmoscopy (AOSLO) and high-magnification scanning laser ophthalmoscopy, can be employed to capture retinal image data for the computation of cone photoreceptor metrics. AOSLO systems are not currently approved for clinical applications in the United States but are used world-wide in academic settings in clinical research. High resolution commercial imaging systems such as the Imagine Eyes rtx1 camera and the Heidelberg Spectralis with HiMag lens can be used for cellular imaging of the retina, although these systems may not have specific regulatory clearance for photoreceptor quantification. The integration of these imaging technologies with the disclosed cone photoreceptor metrics can provide improved methods for patient stratification and trial management in the context of retinal degenerative diseases.

The disclosed techniques provide a data-driven approach to patient selection and stratification in clinical trials for degenerative retinal diseases. By utilizing cone photoreceptor metrics derived from advanced retinal imaging, these methods can enable the identification of patients with specific therapeutic potential based on the progression of retinal degeneration. This approach can result in more precise and effective clinical trials by leveraging objective biomarkers for patient selection and treatment evaluation.

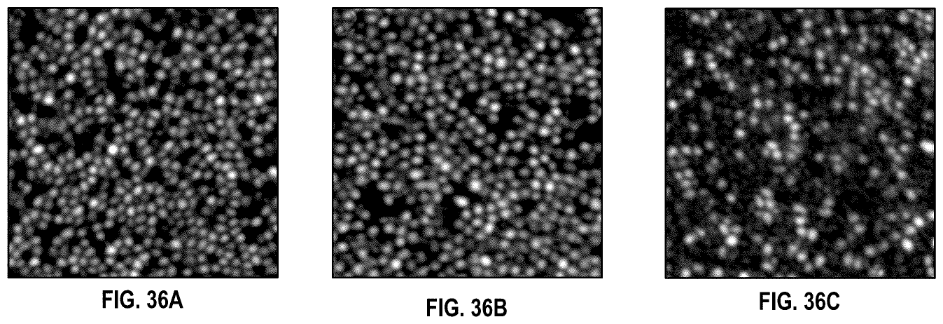

FIGS. 36A-36C depict retinal images representing cone photoreceptor topography at varying levels of structural integrity, which can be utilized to compute quantitative metrics related to cone photoreceptor distribution. These figures illustrate the changes in cone photoreceptor arrangement across different stages of retinal degeneration.

FIG. 36A represents a retinal image with a relatively uniform cone photoreceptor distribution, indicative of a healthy retina. The dense and regular arrangement of cone photoreceptors in this figure may correspond to a high cone density metric and a low cone spacing metric. The regularity of the cone photoreceptor packing may be reflected in the regularity metric, indicating minimal disruption in cone arrangement.

FIG. 36B shows a retinal image with mild to moderate disruption in cone photoreceptor topography. The cone density metric may be lower than that of FIG. 36A, and the cone spacing metric may reflect increased distances between adjacent cones. The regularity metric may also indicate a decrease in the uniformity of cone photoreceptor packing. This image may correspond to an early or intermediate stage of retinal degeneration, which can be used to stratify individuals based on disease progression.

FIG. 36C illustrates a retinal image with significant disruption in cone photoreceptor distribution, indicative of advanced retinal degeneration. The cone density metric is substantially reduced, and the cone spacing metric shows increased irregularity in the arrangement of photoreceptors. The regularity metric may reflect a high degree of variability in cone-to-cone spacing, representing an advanced stage of degeneration. This image can be used to determine exclusion from certain clinical trials or identify individuals at later stages of retinal disease progression.

FIGS. 36A-36C demonstrate different levels of cone photoreceptor packing and spacing, which can be used to assess retinal health and identify various stages of retinal degeneration. The observed patterns of cone density and photoreceptor arrangement provide valuable insights into the structural integrity of the retina. By comparing these metrics with predefined thresholds, individuals can be stratified based on the severity of retinal degeneration, aiding in the determination of eligibility for participation in clinical trials for treating degenerative retinal diseases.

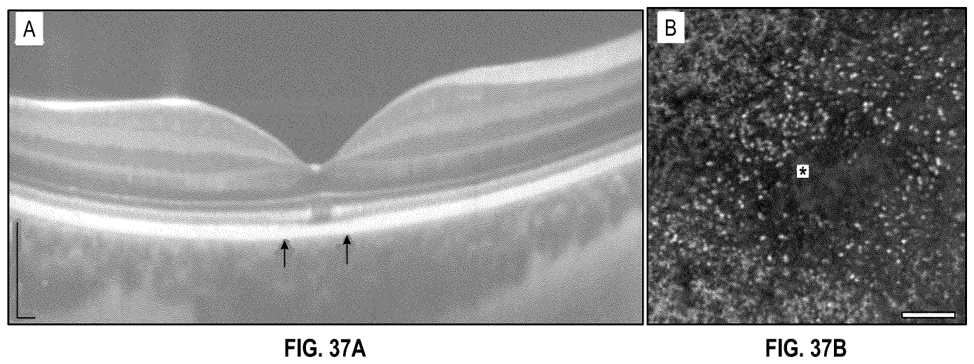

FIG. 37A depicts a cross-sectional optical coherence tomography (OCT) image of the retina, highlighting the structural layers of the retina in a subject with Blue Cone Monochromacy (BCM). The image illustrates a zone devoid of S-cones (indicated by arrows), located in the central macular region. The foveal depression and surrounding retinal layers are clearly visible, demonstrating the structural integrity of the retinal layers despite the absence of S-cones. This figure may be used to provide a visual representation of cone photoreceptor abnormalities associated with retinal degenerative conditions, particularly highlighting the selective loss or absence of specific cone types.

FIG. 37B shows a high-resolution retinal image captured using adaptive optics (AO) imaging. The image depicts the spatial distribution of cone photoreceptors in the retina of the same subject shown in FIG. 37A, specifically in the S-cone free zone. The asterisk marks the center of the fovea, and the surrounding cone mosaic is displayed with irregular spacing and reduced density compared to a typical cone distribution. This image provides further insight into the disrupted cone topography in patients with BCM, supporting the analysis of cone density and spacing metrics for use in clinical trial stratification.

FIGS. 38A and 38B depict high-resolution adaptive optics (AO) retinal images of two patients with achromatopsia, a condition characterized by the absence of cone function. Despite being clinically identical, with no measurable cone function, the images reveal variability in the remnant cone structure between the patients.

FIG. 38A shows a retinal image from a patient with mutations in the CNGB3 gene (c.1148delC, p.Thr383fs and c.983T>A, p.Met328Lys), illustrating a sparse and irregular distribution of remnant cones. The image captures a region where cone density is significantly reduced, with the remaining cones exhibiting an irregular spatial arrangement. The low density and scattered distribution of cones can be indicative of advanced retinal degeneration.

FIG. 38B shows a retinal image from a second patient with a different set of mutations in the CNGB3 gene (c.1148delC, p.Thr383fs and c.1255G>T, p.Glu419stop). This image reveals a relatively more organized cone mosaic, with a higher density of cones compared to FIG. 38A. Although cone function is absent in both patients, the variability in cone structure is apparent, with the cones in this image being more closely packed and arranged in a more uniform pattern.

These figures illustrate the variability in cone photoreceptor structure between patients with achromatopsia, despite the absence of cone function. This variability in remnant cone structure can be used as a metric for patient stratification in clinical trials, supporting the development of tailored therapeutic approaches based on individual retinal architecture.

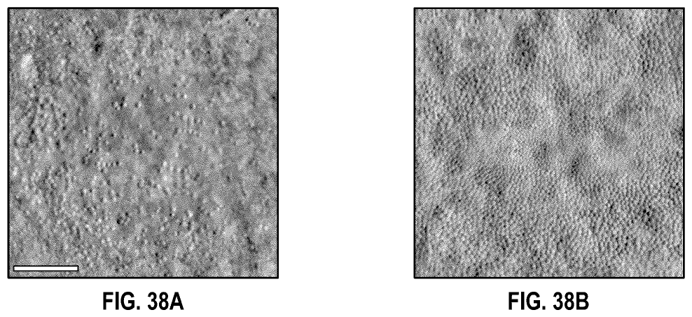

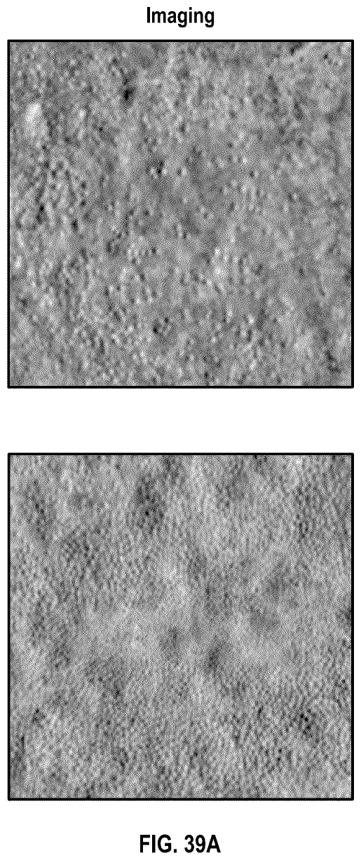

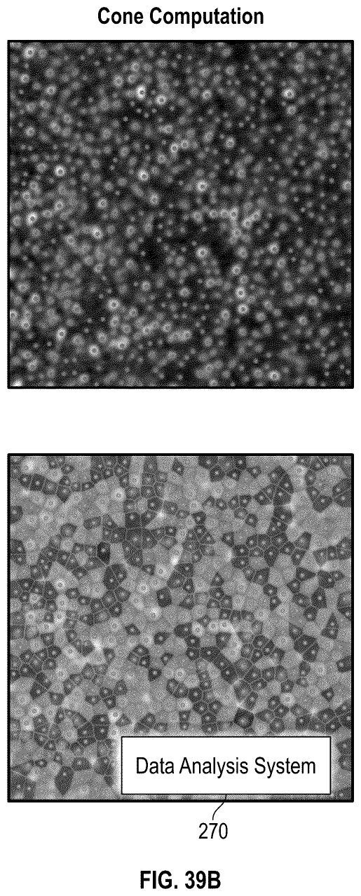

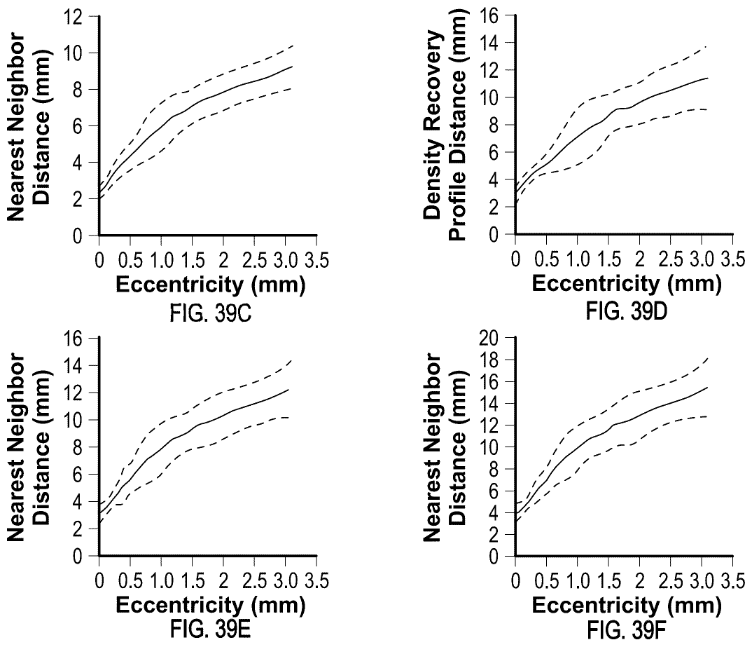

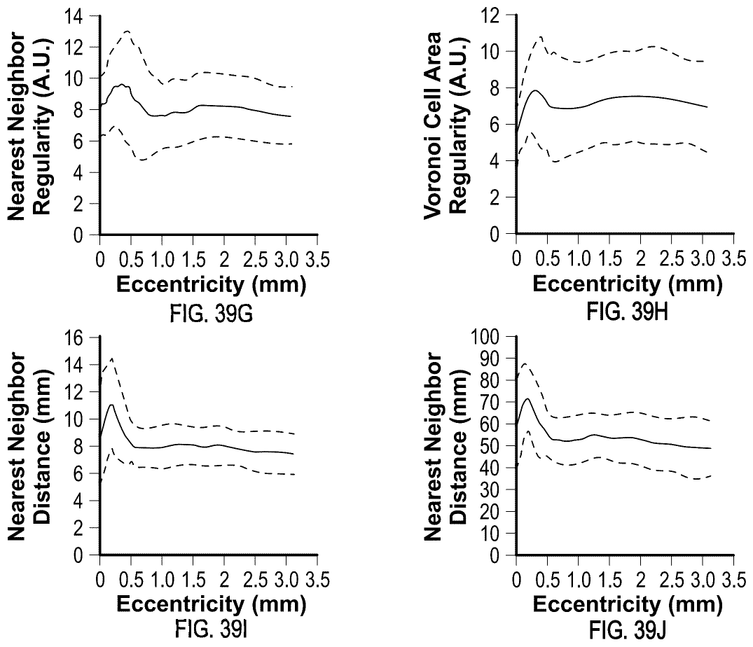

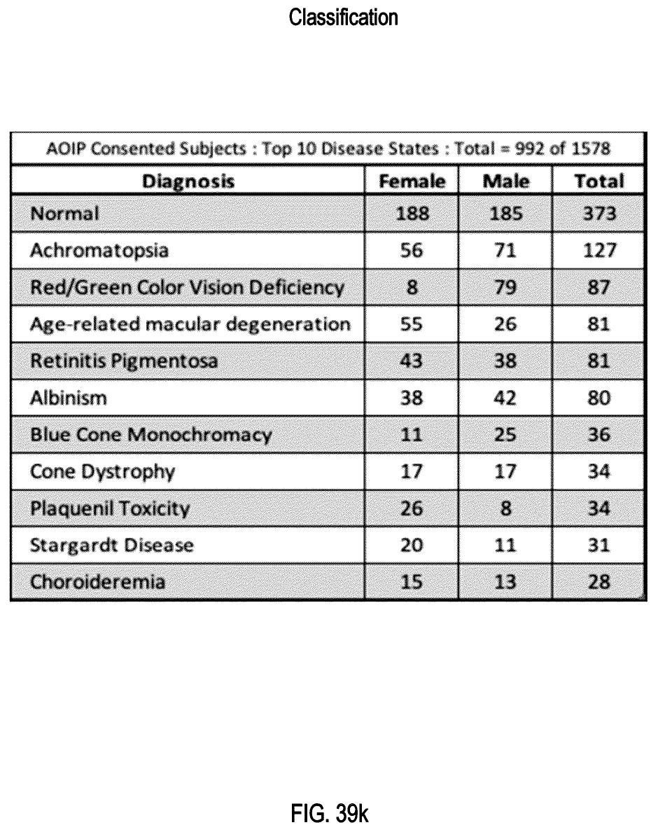

FIGS. 39A-39K illustrate different stages of the biomarker development process for analyzing cone photoreceptor topography, progressing from imaging through computation and classification to final analysis.

FIG. 39A displays two high-resolution retinal images generated using adaptive optics (AO) imaging. These images capture the spatial arrangement and distribution of cone photoreceptors within the retina for two patients who present identically in a clinical evaluation. As evidenced in the AO images, the patient reflected in the top image possesses a more irregular and sparse cone photoreceptor distribution, while the patient reflected in the bottom image displays a relatively uniform and denser cone mosaic. The second patient has greater therapeutic potential. These images represent an initial step in the biomarker development process, where retinal images are obtained for subsequent analysis.

FIG. 39B shows the results of cone computation derived from the retinal images captured in FIG. 39A. The top image illustrates a cone detection algorithm applied to the retinal data, where individual cone photoreceptors are highlighted for analysis. The bottom image demonstrates a computational representation of cone packing and distribution using the ocuLytics™ tool, visualizing cone regularity, density, and spacing patterns in a Voronoi diagram. This step in the process provides quantitative data for assessing retinal structure and health.

FIGS. 39C-K depict the analysis stage, where various metrics related to cone density and spacing are computed and visualized. Typical “normal” values of eight key spatial metrics are plotted as a function of eccentricity from the fovea, including nearest neighbor spacing (NND), inter-cell spacing (ICD), and Voronoi area regularity (VCAR). These metrics are used to classify the retinal structure based on cone photoreceptor health and distribution, supporting the identification of retinal degeneration patterns.

FIGS. 39C-K depict various graphs representing photoreceptor spatial metrics as a function of eccentricity (distance from the fovea) for characterizing the spatial distribution of cone photoreceptors in the retina. The metrics include measurements of spacing and regularity, providing insight into the retinal structure across different eccentricities.

In each graph, the solid lines represent the mean or average value for each respective photoreceptor spatial metric as a function of eccentricity. The dashed lines illustrate the variability around the mean, such as confidence intervals or standard deviations, indicating the range within which the majority of data points are expected to fall. This visualization provides insight into the general trends of the metrics and the variability in retinal structure among different subjects or measurements.

These graphs collectively represent metrics for characterizing the spatial distribution and regularity of cone photoreceptors in the retina. The variations in these metrics as a function of eccentricity provide valuable data for assessing retinal health and degeneration, particularly in clinical trials focused on retinal diseases.

FIG. 39C illustrates the Nearest Neighbor Distance (NND) plotted against eccentricity. This metric represents the average distance between a cone photoreceptor and its nearest neighboring cone, increasing with greater eccentricity from the fovea.

FIG. 39D shows the Density Recovery Profile (DRP), representing the arrangement of photoreceptors as a function of eccentricity. This profile reflects how the cone density changes with distance from the fovea.

FIG. 39E illustrates the Nearest Distance (ND) metric, which measures the shortest distance between cone photoreceptors across different eccentricities.

FIG. 39F displays the Furthest Neighbor Distance (FND), which quantifies the distance to the furthest neighboring cone within a given region. This metric also increases as the distance from the fovea grows.

FIG. 39G shows the Nearest Neighbor Regularity (NNR), which quantifies the regularity of spacing between neighboring cones. A decrease in regularity can be observed as eccentricity increases.

FIG. 39H illustrates the Voronoi Cell Area Regularity (VCAR), a measure of the geometric regularity of the cone photoreceptor mosaic. This metric provides insight into the consistency of cone arrangement as a function of eccentricity.

FIG. 39I shows the Number of Neighbors Regularity (NoNR), representing the uniformity in the number of neighboring photoreceptors for each cone. The variation in the number of neighbors decreases as eccentricity increases.

FIG. 39J presents the Percent Six-Sided (Percent Six-Sided Voronoi cells), which measures the percentage of cone photoreceptors with six neighboring cones, a common geometric configuration in a healthy retinal mosaic. A decline in six-sided configurations can be observed with increasing eccentricity.

FIG. 39K presents the classification stage, where compiled data from multiple subjects are analyzed to identify trends and correlations in cone photoreceptor metrics. The table displays diagnostic categories and patient distribution for a retrospective study conducted by the authors to establish a baseline classification system for healthy eyes versus diseased eyes in a study of ten cone-implicated conditions such as achromatopsia and age-related macular degeneration (AMD). This analysis enables stratification of patients based on their retinal structure and can inform decisions for clinical trial inclusion and therapeutic potential.

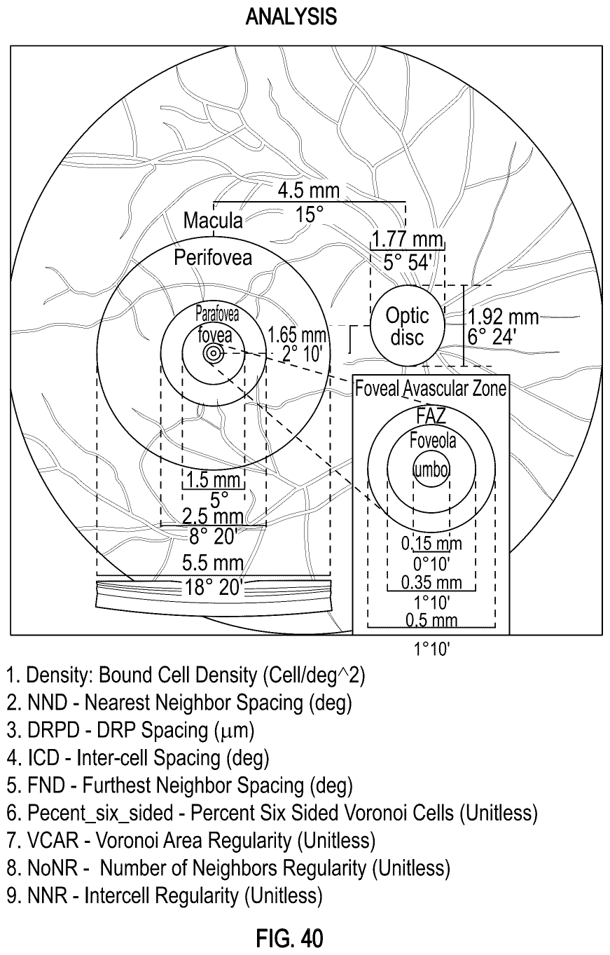

FIG. 40 is an illustration of various retinal domains, with the large circular area outline the macula. The concentric circles are used to define differential areas of visual acuity by their eccentricity values (distance from foveal center measured in milliradians (mrad), degrees (deg), and millimeters (mm)) for specific retinal regions. The table also lists different meridian sectors (superior—towards forehead, nasal—towards nose and optic nerve head, inferior—towards chin, and temporal—towards temple, or ear) used to describe the spatial orientation of each retinal domain.

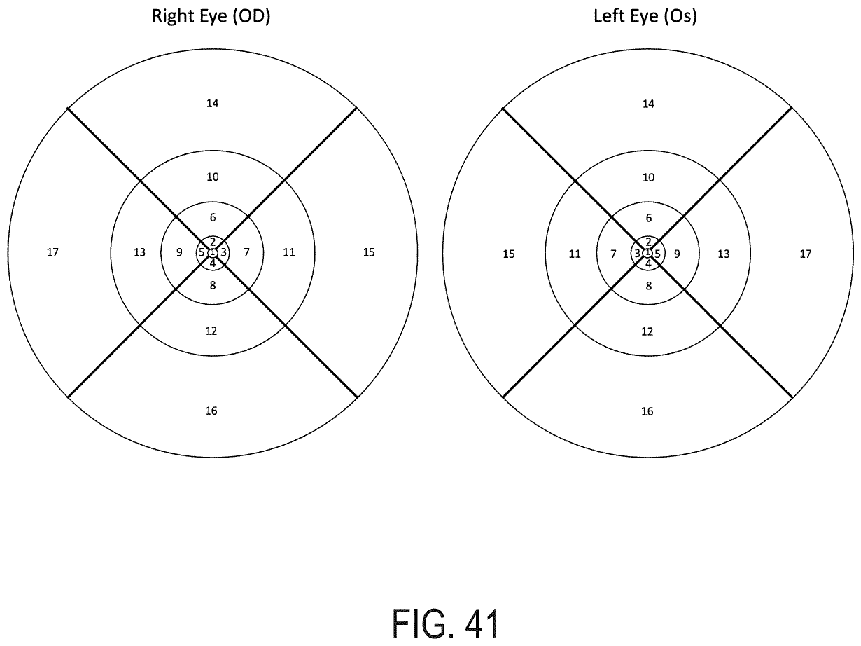

FIG. 41 illustrates a grid in an embodiment of the present invention that is used to localize retinal sectors of interest for analysis of the retinal metrics. This grid is a modification of the commonly used 9 sector ETDRS (Early Treatment Diabetic Retinopathy Study) grid. Our modified grid consists of 17 sectors which, more importantly than providing finer granularity, provides definition to regions that exhibit meaningful transitions in cone density and visual acuity.

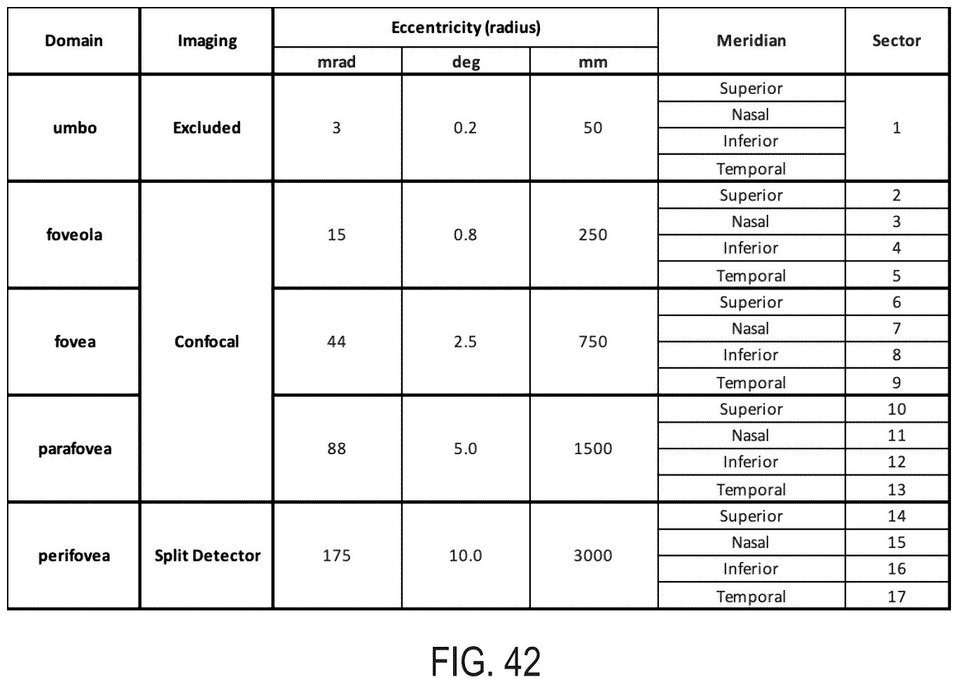

The table of FIG. 42 identifies five retinal domains: the umbo, foveola, fovea, parafovea, and perifovea, as a function of eccentricity radius. The umbo is marked as “Excluded” from imaging for lack of cones, with minimal eccentricity values of 3 mrad, 0.2 degrees, and 50 mm. The foveola and fovea are cone-rich regions with the foveola extending to an eccentricity of 15 mrad, 0.8 degrees, and 250 mm, and the fovea extending to an eccentricity of 44 mrad, 2.5 degrees, and 750 mm. The parafovea and perifovea are regions of the macula, contributing to visual perception but with lower cone density and an increase of rod photoceptors. This region is defined by eccentricity values extending to of 88 mrad, 5.0 degrees, 1500 mm for the parafovea, and 175 mrad, 10.0 degrees, 3000 mm for the perifovea.

AOSLO imaging involves multiple detection channels. The Imaging column of FIG. 42 identifies the imaging channel that is most used to visualize cones in the specified regions. This channel identification is informational only and not a specification for application of any techniques herein.

The table also divides the retinal regions into four specific meridian, and numbers the complete set of sectors. The umbo is assigned to sector 1, while the foveola is associated with sectors 2 through 5, the fovea with sectors 6 through 9, the parafovea with sectors 10 through 13, and the perifovea with sectors 14 through 17. The sector number of the left eye is the horizontal mirror image of the right eye, such that sector 15 is always the sector closest to the optic nerve head.

This table provides a detailed framework for imaging and analyzing specific retinal regions using different imaging techniques, highlighting the spatial orientation and extent of each retinal domain based on eccentricity and meridian sector.

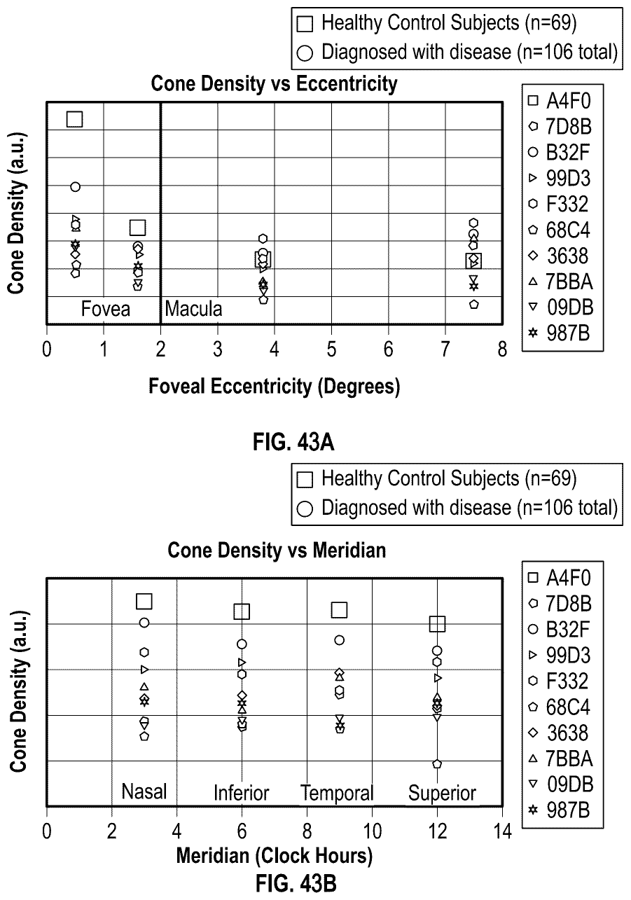

FIGS. 43A-47B illustrate key results from our retrospective study on markers of various cone mediated diseases, with examples of photoreceptor metrics and their spatial distribution as functions of both foveal eccentricity and meridian sector, for healthy controls and individuals diagnosed with retinal diseases. The figures represent metrics and localizations that can be used to assess retinal structure and photoreceptor topography, including cone density, nearest neighbor spacing, regularity, and cone packing.

FIGS. 43A and 43B present Cone Density Distribution as a function of foveal eccentricity (FIG. 43A) and meridian (FIG. 43B). The y-axis represents cone density (measured in arbitrary units), with data points differentiated between healthy control subjects (indicated by open squares) and those diagnosed with various diseases (indicated by colored circles). These plots show how cone density varies across the retinal structure, particularly as the distance from the fovea increases or along different meridian clock hours. A clear distinction can be observed between healthy retina cone densities in the fovea compared to retinas with disease, with less specific differentiation in the outer macula. Additionally, a gradient can be observed in the healthy retina, from peak values in the foveola to decreasing values in the outer fovea and macula.

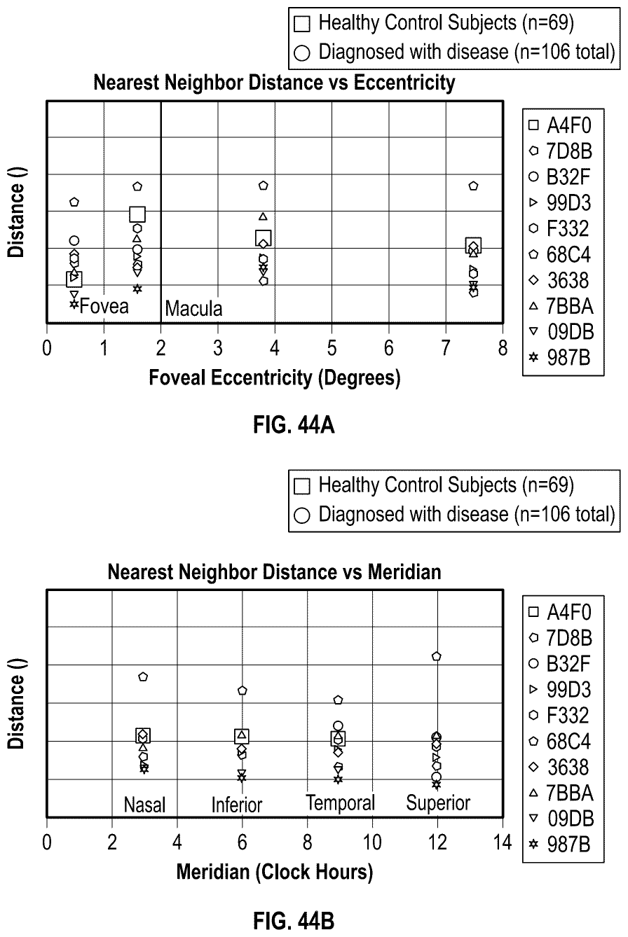

FIGS. 44A and 44B show Nearest Neighbor Spacing, defined as the distance between adjacent cone photoreceptors, plotted against foveal eccentricity (FIG. 44A) and meridian (FIG. 44B). The y-axis represents the measured distance between neighboring photoreceptors, with comparisons drawn between healthy control subjects and individuals diagnosed with retinal disease. These figures provide insight into the uniformity of cone distribution across the retina. Note the inverse relationship between cone density and nearest neighbor distance in healthy retina.

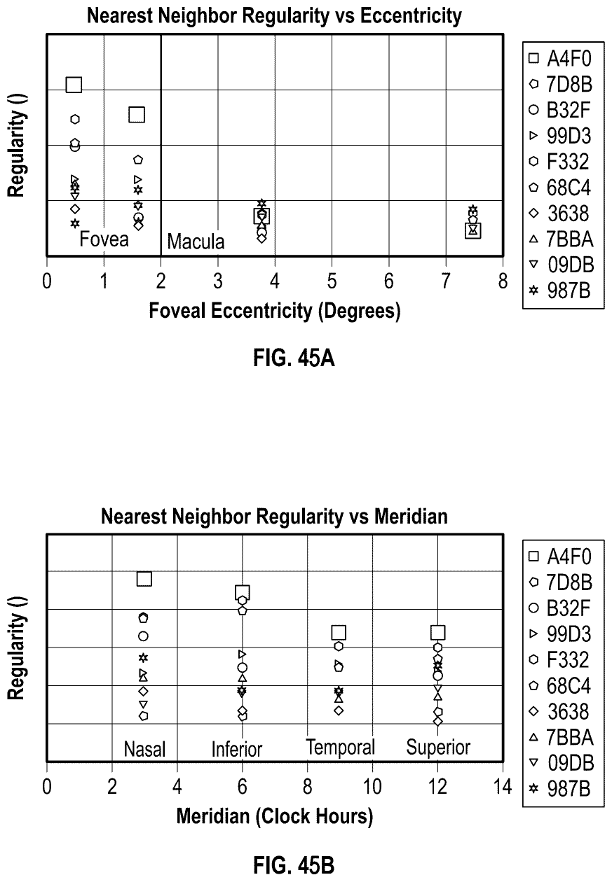

FIGS. 45A and 45B depict Nearest Neighbor Regularity, which quantifies the consistency of spacing between neighboring cones. Regularity, the inverse of coefficient of variation, is plotted as a function of foveal eccentricity (FIG. 45A) and meridian (FIG. 45B), with regularity values indicating the variable arrangement of cones. Data for healthy controls and diagnosed individuals are displayed, highlighting differences in regularity across retinal regions. Note the reduction in regularity of cone spacing in fovea with disease.

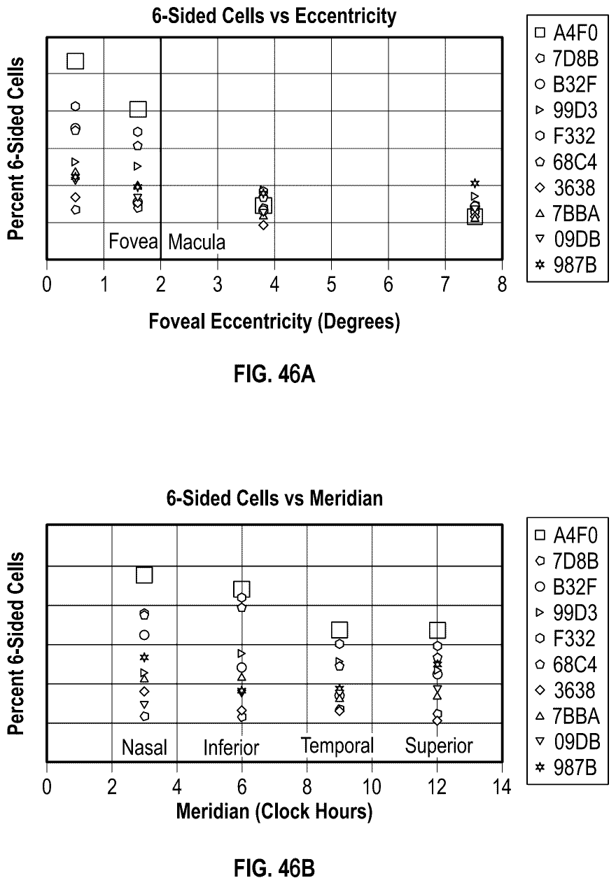

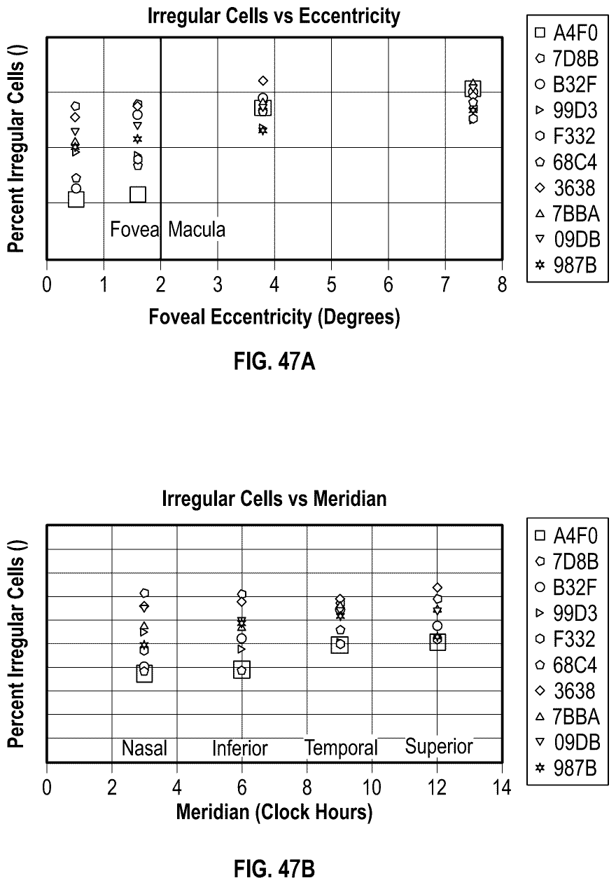

FIGS. 46 and 47 show Cone Packing, expressed as the Fraction of Six-Sided Cells expected in hexagonal close-packing geometries (FIG. 46A-46B) and Fraction of Irregular Cells (less than 5-sided or greater than 7-sided) (FIG. 47A-47B), plotted as a function of both foveal eccentricity and meridian sector. These figures illustrate the percentage of cones exhibiting specific geometric configurations, providing insights into the structural integrity and arrangement of cone photoreceptors. Healthy controls and diagnosed subjects are compared to demonstrate how cone packing may be altered in retinal diseases. Note that the packing geometry elucidates new insights on the circumferential variations (e.g. by meridian).

These figures collectively illustrate how quantitative metrics related to cone photoreceptor distribution can be used to assess retinal health and degeneration. The variability in these metrics across different retinal regions and between healthy and diagnosed individuals can provide valuable information for patient stratification in clinical trials and for evaluating the progression of retinal diseases.

A Principal Components Analysis (PCA) can be used to generate reduced features that have the greatest power in distinguishing health from diseased eyes, one disease state from another, or one stage of disease from another. The sensitivity to detection will always depend on the disease, state of disease, metrics, and regions included or excluded in analysis. The metrics may be regionalized, and the regionalization may be used to increase the specificity of the biomarker. For example, the metric may be defined as “cone density in the fovea” in contrast to “cone density in the macula,” or “cone density in sector 7 (nasal fovea)” in contrast to “cone density in sector 15 (nasal perifovea)”. Any such combinations of metric and location that has a basis in the disease pathogenesis will increase the classification accuracy and predictive strength of the biomarker.

The data ontology in the workflow allows testing along all dimensions of interest to isolate the combinations that are both most sensitive to state variations, and most specific to a given state. As such, the workflow processes are configurable to include, record, and trace multiple test configurations, classification models, and statistical hypothesis tests in a batch mode to rapidly generate a set of candidate imaging biomarkers with statistical tests of sensitivity and specificity.

A unique aspect of the inventive biomarker discovery process is the generation of biomarkers that comprise at least two weakly correlated metrics, and at least two distinct regions. The definition of weakly-correlated is one of choice; clearly cone counts and cone density are not weakly correlated, while Nearest Neighbor Distance and Percent 6-Sided Cells are weakly correlated. Biomarkers that combine two such weakly correlated metrics from two distinct regions (for example foveola and fovea, or fovea and macula) will exhibit the greatest specificity to specific states, and therefore lead to tighter inclusion criteria when selecting patients based on state of disease.

### Flow Diagram Retinal Imaging and Clinical Trial Eligibility

Clinical trials for treating degenerative retinal diseases can benefit from precise patient selection to identify individuals with specific therapeutic potential. Conventional patient selection methods may not fully leverage available retinal imaging data, potentially leading to inefficiencies in trial outcomes. A more data-driven approach, based on quantitative biomarkers derived from retinal imaging, may enhance the accuracy of eligibility assessments and optimize the trial process. The inventive concepts described herein disclose a method for determining an individual's eligibility for clinical trials using cone photoreceptor metrics obtained from retinal imaging data.

The disclosed inventive concepts include obtaining retinal image data from an individual's eye, which reflects the topographic structure of cone photoreceptors. This data can be analyzed to compute various quantitative metrics related to cone photoreceptor distribution, such as cone density, cone spacing, or the regularity of cone packing. These metrics can be compared to predefined thresholds indicative of retinal degeneration progression, allowing for the stratification of patients into inclusion or exclusion categories for clinical trials.

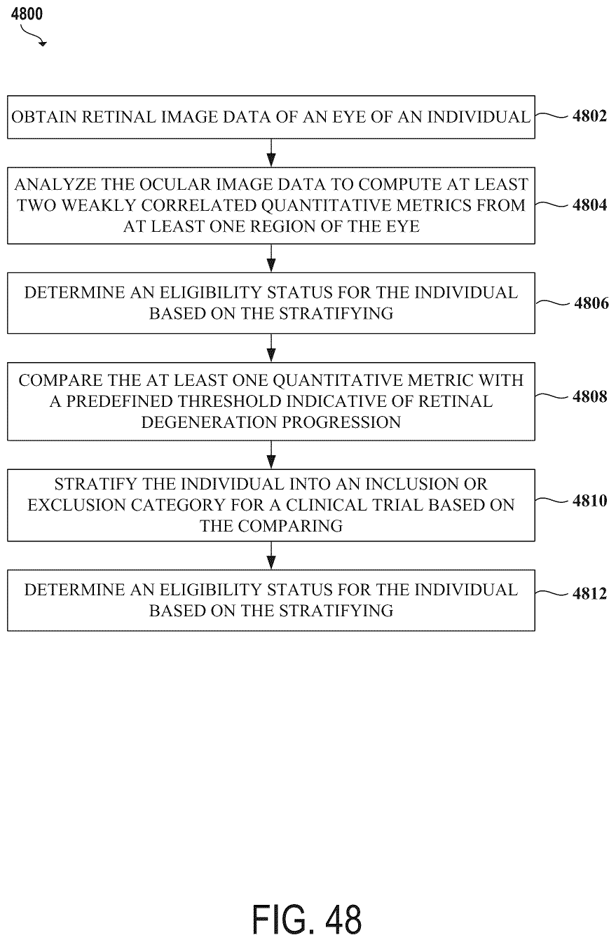

FIG. 48 is a flow diagram illustrative of an embodiment of a routine 4800 for determining the eligibility of an individual for a clinical trial. Although described as being implemented by the workflow coordinator 220, it will be understood that the elements outlined for routine 4800 can be implemented by any one or a combination of computing devices/components that are associated with the workflow environment 200, such as but not limited to the data intake system 210, the data store system 230, the visualization interface system 260, the data analysis system 270, or the reporting system 280. Thus, the following illustrative embodiment should not be construed as limiting.

At block 4802, the system can be configured to obtain retinal image data from the eye of an individual. This data reflects the topographic structure of cone photoreceptors within the retina, which serves as a foundational element for further analysis. In some cases, the retinal image data can be obtained through advanced imaging technologies such as adaptive optics-enhanced scanning laser ophthalmoscopy (AOSLO) or confocal scanning laser ophthalmoscopy (SLO). These imaging methods can capture high-resolution images of the cone photoreceptor mosaic, providing detailed insights into the distribution and structure of cone cells within the retina. The captured data can be stored for subsequent analysis and evaluation.

At block 4804, the workflow coordinator 220 analyzes the ocular image data to compute at least two weakly correlated quantitative metrics from at least one region of the eye. This analysis can involve various metrics such as cone density, cone spacing, or the regularity of cone packing. For instance, the cone density metric can be calculated as a function of distance from the fovea, which is the central region of the retina. The system can also determine the spacing between adjacent cone photoreceptors or assess how regularly the cones are packed. In some cases, a convolutional neural network (CNN) trained on retinal datasets may be employed to detect the locations of individual cone photoreceptors, enabling automated and efficient computation of these metrics. This allows for a more detailed and quantitative assessment of retinal health. For increased specificity of a resultant biomarker, at least two weakly-correlated quantitative metrics may be combined into a composite metric, for example through PCA. Further, the metrics may be regionalized, and the regionalization may be used to further increase the specificity of the biomarker. For example, the metric may be defined as “cone density in the fovea” in contrast to “cone density in the macula,” or “cone density in sector 7 (nasal fovea)” in contrast to “cone density in sector 15 (nasal perifovea).” Any such combinations of metric and location that have a basis in the disease pathogenesis will increase the classification accuracy and predictive strength of the biomarker.

At block 4806, the system determines an eligibility status for the individual based on the stratifying process. The stratifying process categorizes individuals according to the severity of their condition using the quantitative metrics analyzed in the previous step. These metrics may indicate the stage of retinal degeneration or other health indicators. Based on the analysis, the system assigns an eligibility status (e.g., likely eligible or not eligible) as a foundational step for further comparison with predefined thresholds. This status can be updated after further evaluation.

At block 4808, the workflow coordinator 220 compares the at least one quantitative metric with predefined thresholds indicative of retinal degeneration progression. These thresholds can be based on a normative dataset of healthy individuals or established clinical criteria that reflect the different stages of retinal diseases such as retinitis pigmentosa or age-related macular degeneration. For example, a significant decrease in cone density or an irregularity in cone packing may indicate the presence of retinal degeneration. By comparing the patient's metrics against these thresholds, the system can assess the severity of the disease and the likelihood of therapeutic success in clinical trials.

At block 4810, the workflow coordinator 220 stratifies the individual into either an inclusion or exclusion category for a clinical trial based on the comparison of the computed metrics with the predefined thresholds. In some cases, the stratification process can involve further refinement, where individuals are grouped based on the severity of their condition, allowing for more targeted inclusion in clinical trials that match their disease stage. For example, patients with early-stage degeneration may be included in trials aimed at preventing progression, while those with more advanced disease may be better suited for trials focused on regeneration or vision restoration.

At block 4812, the workflow coordinator 220 determines the eligibility status for the individual based on the stratifying. The eligibility determination can be influenced by whether the individual's cone photoreceptor metrics meet or exceed the predefined thresholds. In some cases, additional factors such as the progression rate of the disease or the presence of localized areas of degeneration, as represented by spatial heat maps, may also be considered in determining eligibility. If the individual's metrics suggest therapeutic potential, the system may classify the individual as eligible for inclusion in the clinical trial, thereby improving the precision of patient selection and enhancing the likelihood of trial success.

A closely related application of this inventive approach is to select patients as appropriate candidates for a treatment. The same criteria for including or excluding patients for a clinical trial may be used to establish a patient's eligibility to receive a treatment with the therapy.

Based on results of the clinical trial or post-market surveillance, the criteria for selecting eligible patients using the inventive biomarker approach may be tightened or loosened or extended to a new intended use. Application of such eligibility requirements should greatly improve the probability of a successful outcome for a patient.

This inventive approach may also be used diagnostically to grade disease stage and establish recommendations for referrals or specific protocols of care.

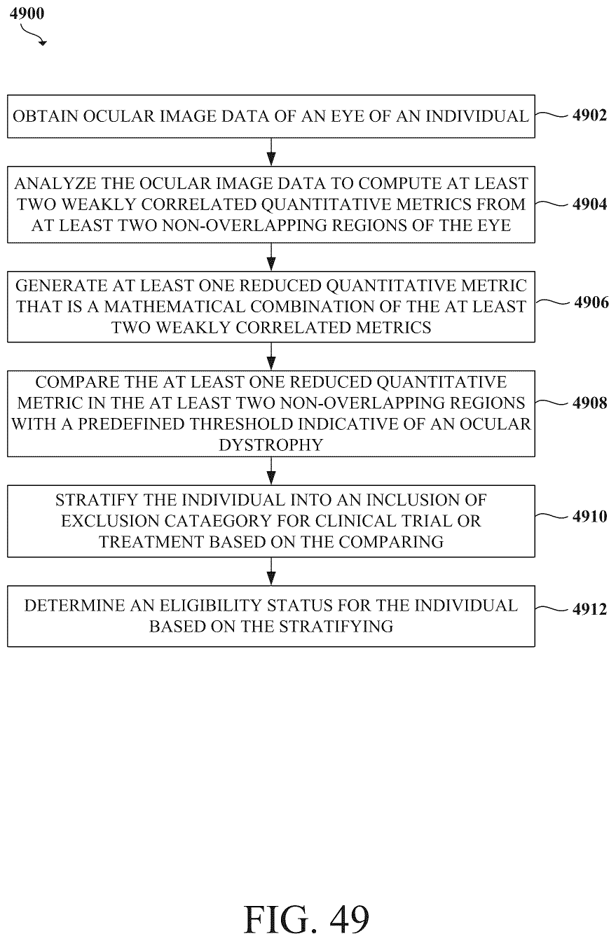

FIG. 49 is a flow diagram illustrative of an embodiment of a routine 4900 for determining the eligibility of an individual for a clinical trial or treatment. Although described as being implemented by the workflow coordinator 220, it will be understood that the elements outlined for routine 4900 can be implemented by any one or a combination of computing devices/components that are associated with the workflow environment 200, such as but not limited to the data intake system 210, the data store system 230, the visualization interface system 260, the data analysis system 270, or the reporting system 280. Thus, the following illustrative embodiment should not be construed as limiting.

At block 4902, the system can be configured to obtain ocular image data from the eye of an individual. This data reflects the topographic structure of cone photoreceptors within the retina, which serves as a foundational element for further analysis. In some cases, the ocular image data can be obtained through advanced imaging technologies such as adaptive optics-enhanced scanning laser ophthalmoscopy (AOSLO) or confocal scanning laser ophthalmoscopy (SLO). These imaging methods can capture high-resolution images of the cone photoreceptor mosaic, providing detailed insights into the distribution and structure of cone cells within the retina. The captured data can be stored for subsequent analysis and evaluation.

At block 4904, the workflow coordinator 220 analyzes the ocular image data to compute at least two weakly correlated quantitative metrics from at least two non-overlapping regions of the eye. This analysis can involve metrics such as cone density, cone spacing, or the regularity of cone packing. For instance, the cone density metric may be calculated as a function of distance from the fovea, the central region of the retina. The system can also determine the spacing between adjacent cone photoreceptors or assess the regularity of cone packing. In some cases, a convolutional neural network (CNN) trained on retinal datasets may be employed to detect the locations of individual cone photoreceptors, enabling automated and efficient computation of these metrics.

At block 4906, the system generates at least one reduced quantitative metric that is a mathematical combination of the at least two weakly correlated metrics from non-overlapping regions of the eye. These reduced metrics provide more refined insights into the condition of the retina and can be utilized for comparison with predefined thresholds for assessing ocular diseases such as retinal dystrophies.

At block 4908, the system compares the at least one reduced quantitative metric in the at least two non-overlapping regions with a predefined threshold indicative of an ocular dystrophy. These thresholds may be derived from normative datasets or based on established clinical criteria reflecting different stages of retinal diseases such as retinitis pigmentosa or age-related macular degeneration. Significant deviations from the threshold may indicate the presence of retinal degeneration or other forms of ocular dystrophy.

At block 4910, the workflow coordinator 220 stratifies the individual into an inclusion or exclusion category for a clinical trial or treatment based on the comparison of the computed metrics with the predefined thresholds. This stratification can involve further refinement, where individuals are grouped based on the severity of their condition, allowing for more targeted inclusion in clinical trials or treatments that align with their disease stage.

At block 4912, the system determines the eligibility status of the individual based on the stratifying process. The eligibility determination can be influenced by whether the individual's reduced metrics meet or exceed the predefined thresholds. Additional factors, such as the progression rate of the disease or localized areas of degeneration as represented by spatial heat maps, may also be considered. If the individual's metrics suggest therapeutic potential, the system may classify the individual as eligible for a clinical trial or treatment, enhancing the likelihood of successful outcomes.

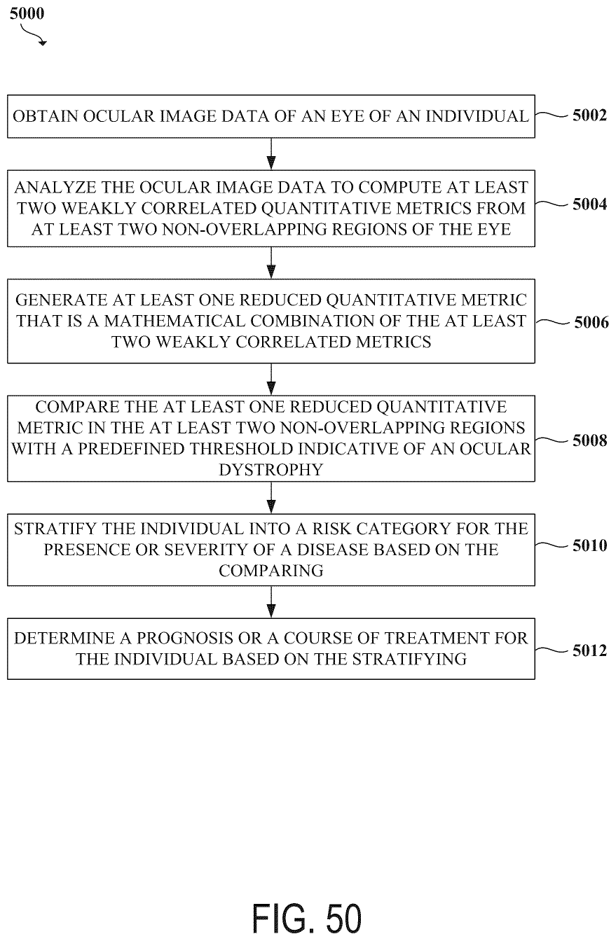

FIG. 50 is a flow diagram illustrative of an embodiment of a routine 5000 for determining the prognosis or course of treatment for an individual based on ocular image data. Although described as being implemented by the workflow coordinator 220, it will be understood that the elements outlined for routine 5000 can be implemented by any one or a combination of computing devices/components associated with the workflow environment 200, such as but not limited to the data intake system 210, the data store system 230, the visualization interface system 260, the data analysis system 270, or the reporting system 280. The following illustrative embodiment should not be construed as limiting.

At block 5002, the system is configured to obtain ocular image data from the eye of an individual. This data reflects the topographic structure of cone photoreceptors within the retina, serving as a foundational element for further analysis. In some cases, the ocular image data can be obtained through advanced imaging technologies such as adaptive optics-enhanced scanning laser ophthalmoscopy (AOSLO) or confocal scanning laser ophthalmoscopy (SLO). These imaging methods capture high-resolution images of the cone photoreceptor mosaic, providing detailed insights into the distribution and structure of cone cells within the retina. The captured data can be stored for subsequent analysis and evaluation.

At block 5004, the workflow coordinator 220 analyzes the ocular image data to compute at least two weakly correlated quantitative metrics from at least two non-overlapping regions of the eye. This analysis may involve metrics such as cone density, cone spacing, or the regularity of cone packing. For instance, the cone density metric can be calculated as a function of distance from the fovea, the central region of the retina. The system can also determine the spacing between adjacent cone photoreceptors or assess the regularity of cone packing. In some cases, a convolutional neural network (CNN) trained on retinal datasets may be employed to detect the locations of individual cone photoreceptors, enabling automated and efficient computation of these metrics.

At block 5006, the system generates at least one reduced quantitative metric that is a mathematical combination of the at least two weakly correlated metrics from non-overlapping regions of the eye. These reduced metrics provide refined insights into the condition of the retina and are utilized for comparison with predefined thresholds for assessing ocular diseases such as retinal dystrophies.

At block 5008, the system compares the at least one reduced quantitative metric in the at least two non-overlapping regions with a predefined threshold indicative of an ocular dystrophy. These thresholds may be derived from normative datasets or established clinical criteria that reflect the different stages of retinal diseases such as retinitis pigmentosa or age-related macular degeneration. Significant deviations from the threshold may indicate the presence of retinal degeneration or other forms of ocular dystrophy.

At block 5010, the system stratifies the individual into a risk category for the presence or severity of a disease based on the comparison of the computed metrics with the predefined thresholds. Stratification can be further refined to categorize individuals based on the severity of their condition, allowing for more accurate predictions of disease progression or risk.

At block 5012, the workflow coordinator 220 determines a prognosis or a course of treatment for the individual based on the stratifying process. The treatment recommendations or prognosis can be influenced by whether the individual's reduced metrics meet or exceed the predefined thresholds. Additional factors, such as the progression rate of the disease or localized areas of degeneration as represented by spatial heat maps, may also be considered when determining the appropriate course of treatment or providing a prognosis for the individual.

### Terminology

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may include, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

As will be appreciated by one of skill in the art, the inventive concept may be embodied as a method, data processing system, or computer program product. Accordingly, the present inventive concept may take the form of an entirely hardware embodiment or an embodiment combining software and hardware aspects all generally referred to herein as a “circuit” or “module.” Furthermore, the present inventive concept may take the form of a computer program product on a computer-usable storage medium having computer-usable program code embodied in the medium. Any suitable computer readable medium may be utilized including hard disks, CD-ROMs, optical storage devices, a transmission media such as those supporting the Internet or an intranet, or magnetic storage devices.

Computer program code for carrying out operations of the present inventive concept may be written in an object-oriented programming language such as Java®, Smalltalk, C++, MATLAB or Python. However, the computer program code for carrying out operations of the present inventive concept may also be written in conventional procedural programming languages, such as the “C” programming language or in a visually oriented programming environment, such as Visual Basic or JavaFX.

The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

The inventive concept is described herein with reference to a flowchart illustration and/or block diagrams of methods, systems and computer program products according to embodiments of the inventive concept. It will be understood that each block of the illustrations, and combinations of blocks, can be implemented by computer program instructions. These computer program instructions may be provided to a processor of a general purpose computer, special purpose computer, a graphics processing unit (GPU), or other programmable data processing apparatus to produce a machine, such that the instructions, which execute via the processor of the computer, a graphics processing unit, or other programmable data processing apparatus, create means for implementing the functions/acts specified in the block or blocks.

These computer program instructions may be stored in a computer-readable memory that can direct a computer or other programmable data processing apparatus to function in a particular manner, such that the instructions stored in the computer-readable memory produce an article of manufacture including instruction means which implement the function/act specified in the block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for managing a configurable medical data processing workflow, the method comprising:
providing a medical data processing workflow comprising a set of configurable processing operations, wherein the medical data processing workflow is applied to process medical imaging data;
configuring one or more processing operations of the set of configurable processing operations based on user-defined criteria, wherein the user-defined criteria comprises at least one of:
a selection of at least one processing operation from a plurality of predefined processing operations,
an indication of an adjustment of one or more parameters associated with the selected processing operation, or
an indication of a user-defined processing operation;
facilitating execution of the set of configurable processing operations on the medical imaging data to generate an output, wherein the execution includes performing a plurality of tasks, the plurality of tasks comprising at least one computer-automated task and at least one operator-assisted task, wherein each task of the plurality of tasks results in at least one of a transformation or an annotation of at least a portion of the medical imaging data;
generating a record for each task of the plurality of tasks, wherein each record includes a unique identifier corresponding to the task and details of a respective transformation or annotation applied to the medical imaging data; and
generating a traceability report based on the plurality of records, wherein the traceability report provides a complete record of all processing operations performed on the medical imaging data, including a sequence of operations, unique identifiers for each operation, details of any transformation or annotation applied to the medical imaging data, and identification of the operator responsible for each task, such that the report enables the tracing of each modification or annotation back to its corresponding step in the medical data processing workflow, ensuring that the entire data processing path is documented and verifiable.

2. The method of claim 1, wherein the set of configurable processing operations and user-defined criteria form an ontology, the ontology comprising a structured framework that defines relationships between data entities.

3. The method of claim 1, wherein the configuring of the one or more processing operations comprises user-configuration through modular low-code or no-code interaction, the modular low-code or no-code interaction including graphical user interface elements associated with modules of an ontology, enabling selection and/or adjustment of workflow operations without requiring detailed coding.

4. The method of claim 1, wherein each configurable processing operation is assigned a universally unique identifier (UUID) and is stored as a reusable configuration, such that the processing operation can be reapplied in subsequent workflows.

5. The method of claim 1, wherein a complete set of user-configurations that form the medical data processing workflow is assigned a universally unique identifier (UUID), enabling a complete version of the medical data processing workflow to be saved and reused in future instances of medical data processing.

6. The method of claim 1, further comprising causing a display to present graphical user interface elements, each graphical user interface element corresponding to a particular configurable processing operation from the set of configurable processing operations, wherein the graphical user interface elements enable selection, adjustment of parameters, or definition of a user-defined processing operation for inclusion in the medical data processing workflow.

7. The method of claim 1, further comprising facilitating human interaction with the medical data processing workflow through graphical user interface elements, wherein the human interaction includes at least one of reviewing, annotating, or adjusting the configurable processing operations based on clinical or operational criteria, and wherein the human interaction is recorded as part of the traceability report.

8. The method of claim 1, wherein the traceability report further includes audit logs of user interactions, wherein each interaction is logged with a unique identifier and user credentials for full accountability.

9. The method of claim 1, further comprising assigning a universally unique identifier (UUID) to the medical imaging data at an initial stage of the workflow, wherein at each subsequent step of the configurable processing workflow, as the data is transformed, annotated, or divided into sub-portions, each resulting portion or subset of the data is assigned an additional UUID, such that the data forms a branching sequence with a unique identifier at each branch, providing traceability for every division and modification of the data throughout the workflow.

10. The method of claim 1, wherein the medical data processing workflow is applied to evaluate a set of prospective biomarkers, wherein the medical data processing workflow comprises computing a set of metrics associated with a set of locations within an image or a test result, associating the metrics with at least one record of patient metadata, and developing a classification schema for sorting patients by categories of the metadata value using a combination of one or more metrics associated with one or more locations in the test region of the patient.

11. The method of claim 10, wherein the medical data processing workflow comprises computing a set metrics, reducing the set of metrics to a predefined set of one or more biomarkers, applying a candidate patient data set to the biomarker workflow, and classifying the candidate patient eligibility for participation in a clinic trial or eligibility to receive a clinical treatment.

12. The method of claim 1, wherein the medical data processing workflow is configured for reading medical data in a clinical research study or a clinical trial.

13. The method of claim 1, wherein the medical data processing workflow comprises automatedly creating masked and randomized batches of images, annotating the masked and randomized batches of images, automatedly computing a set of metrics from the images in the masked and randomized batches, and automatedly creating a report on computed set of metrics.

14. The method of claim 1, wherein the at least one operator-assisted task comprises at least one of selecting, adjusting, or confirming processing operations.

15. The method of claim 1, wherein the traceability report further comprises a hierarchical structure that organizes each task of the plurality of tasks and any derived sub-tasks based on a parent-child relationship, wherein each task and sub-task is assigned a universally unique identifier, such that the hierarchical structure enables tracking and tracing of the processing operations across multiple branches within the medical data processing workflow.

16. The method of claim 1, wherein the universally unique identifier assigned to each task of the plurality of tasks within the medical data processing workflow is linked to subsequent sub-tasks generated from the transformation or annotation of the medical imaging data, such that a hierarchical structure of the traceability report provides a detailed, reproducible path of all tasks and sub-tasks performed within the workflow.

17. The method of claim 1, wherein the hierarchical structure of the traceability report further enables recreation of a complete medical data processing workflow, such that by following the universally unique identifiers and recorded sequence of tasks, the medical data processing operations can be reproduced to generate an output identical to the original workflow execution.

18. A computer-readable medium storing instructions that, when executed by one or more processors, cause the one or more processors to:
provide a medical data processing workflow comprising a set of configurable processing operations, wherein the medical data processing workflow is applied to process medical imaging data;
configure one or more processing operations of the set of configurable processing operations based on user-defined criteria, wherein the user-defined criteria comprises at least one of:
a selection of at least one processing operation from a plurality of predefined processing operations,
an indication of an adjustment of one or more parameters associated with the selected processing operation, or
an indication of a user-defined processing operation;
facilitate execution of the set of configurable processing operations on the medical imaging data to generate an output, wherein the execution includes performing a plurality of tasks, the plurality of tasks comprising at least one computer-automated task and at least one operator-assisted task, wherein each task of the plurality of tasks results in at least one of a transformation or an annotation of at least a portion of the medical imaging data;
generate a record for each task of the plurality of tasks, wherein each record includes a unique identifier corresponding to the task and details of a respective transformation or annotation applied to the medical imaging data; and
generate a traceability report based on the plurality of records, wherein the traceability report provides a complete record of all processing operations performed on the medical imaging data, including a sequence of operations, unique identifiers for each operation, details of any transformation or annotation applied to the medical imaging data, and identification of the operator responsible for each task, such that the report enables the tracing of each modification or annotation back to its corresponding step in the medical data processing workflow, ensuring that the entire data processing path is documented and verifiable.

19. The computer-readable medium of claim 18, wherein the configuring of the one or more processing operations comprises user-configuration through modular low-code or no-code interaction, the modular low-code or no-code interaction including graphical user interface elements associated with modules of an ontology, enabling selection and/or adjustment of workflow operations without requiring detailed coding.

20. A system comprising one or more processors configured to:
provide a medical data processing workflow comprising a set of configurable processing operations, wherein the medical data processing workflow is applied to process medical imaging data;
configure one or more processing operations of the set of configurable processing operations based on user-defined criteria, wherein the user-defined criteria comprises at least one of:
a selection of at least one processing operation from a plurality of predefined processing operations,
an indication of an adjustment of one or more parameters associated with the selected processing operation, or
an indication of a user-defined processing operation;
facilitate execution of the set of configurable processing operations on the medical imaging data to generate an output, wherein the execution includes performing a plurality of tasks, the plurality of tasks comprising at least one computer-automated task and at least one operator-assisted task, wherein each task of the plurality of tasks results in at least one of a transformation or an annotation of at least a portion of the medical imaging data;
generate a record for each task of the plurality of tasks, wherein each record includes a unique identifier corresponding to the task and details of a respective transformation or annotation applied to the medical imaging data; and
generate a traceability report based on the plurality of records, wherein the traceability report provides a complete record of all processing operations performed on the medical imaging data, including a sequence of operations, unique identifiers for each operation, details of any transformation or annotation applied to the medical imaging data, and identification of the operator responsible for each task, such that the report enables the tracing of each modification or annotation back to its corresponding step in the medical data processing workflow, ensuring that the entire data processing path is documented and verifiable.
